SEQUENCE LISTING (2 sequences)

SEQ ID NO: 1 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 2 - protein, 381 aa, Homo sapiens
HMGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

(19) United States
(12) Patent Application Publication
(10) Pub. No.: US 2024/0336563 A1
(43) Pub. Date: Oct. 10, 2024

(54) G-ALPHA-S INHIBITORS AND USES THEREOF

(71) Applicant: The Regents of the University of California, Oakland, CA (US)

(72) Inventors: Kevan M. Shokat, San Francisco, CA (US); Qi Hu, Hangzhou (CN); Shizhong Dai, San Francisco, CA (US)

(21) Appl. No.: 18/287,634

(22) PCT Filed: Apr. 26, 2022
(86) PCT No.: PCT/US2022/026345

§ 371 Date: Oct. 19, 2023

Related U.S. Application Data
(60) Provisional application No. 63/179,969, filed on Apr. 26, 2021.

(51) Int. Cl. C07C 323/44; A61K 31/165; A61K 31/17; A61K 31/381; A61K 31/41; A61K 31/42; A61K 31/433; A61K 31/505; A61K 31/53; A61K 47/64; A61P 35/00; C07D 213/75; C07D 239/34; C07D 257/06; C07D 261/14; C07D 285/135; C07D 333/70

(52) U.S. Cl. CPC C07C 323/44; A61K 31/165; A61K 31/17; A61K 31/381; A61K 31/41; A61K 31/42; A61K 31/433; A61K 31/505; A61K 31/53; A61K 47/64; A61P 35/00; C07D 213/75; C07D 239/34; C07D 257/06; C07D 261/14; C07D 285/135; C07D 333/70

(57) ABSTRACT

Disclosed herein, inter alia, are G-alpha-s Inhibitors and uses thereof.

### CROSS-REFERENCES TO RELATED APPLICATIONS

This application claims the benefit of U.S. Provisional Application No. 63/179,969, filed Apr. 26, 2021, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant number R01 CA244550 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-707001WO_Sequence_Listing_ST25.TXT, created Apr. 19, 2022, 7,057 bytes, machine format IBM-PC, MS Windows operating system, is hereby incorporated by reference.

### BACKGROUND

Guanine nucleotide-binding protein G, alpha (α) subunit (GNAS) mediates G-protein-coupled receptor (GPCR) signaling, a central mechanism by which cells sense and respond to extracellular stimuli. Multiple human cancer types exhibit recurrent gain-of-function mutations in the pathway, most frequently targeting GNAS. The most lethal tumor type where GNAS is frequently mutated is the intraductal papillary mucinous neoplasm (IPMN), a precursor of invasive pancreatic cancer.

Disclosed herein, inter alia, are solutions to these and other problems in the art.

### BRIEF SUMMARY

In an aspect is provided a compound having the formula:

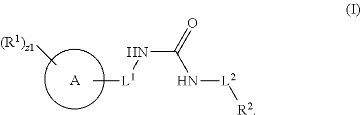

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C (O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

z1 is an integer from 0 to 6.

Ring A is aryl or heteroaryl.

L¹ is a

bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L² is a

bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R² is an electrophilic moiety.

R^{1A}, R^{1B}, R^{1C}, and R^{1D} are independently

hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl.

X and X¹ are independently —F, —Cl, —Br, or —I.

n1 is independently an integer from 0 to 4.

m1 and v1 are independently 1 or 2.

z1 is an integer from 1 to 3. In embodiments, z1 is 0.

In embodiments, Ring A is phenyl or 5 to 6-membered heteroaryl.

In embodiments, the compound has the formula:

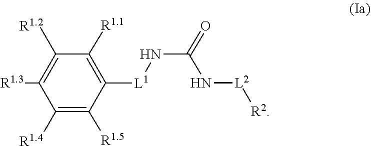

Each R^{1.1}, R^{1.2}, R^{1.3}, R^{1.4}, and R^{1.5} is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B},

—NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C (O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, and R^{1D} are independently

hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl.

X and X¹ are independently —F, —Cl, —Br, or —I.

n1 is independently an integer from 0 to 4.

m1 and v1 are independently 1 or 2.

L¹ is a

bond, —NH—, —O—, —S—, —C(O)—, —C(O)NH—, —NHC(O)—, —NHC(O)NH—, —C(O)O—, —OC(O)—, substituted or unsubstituted alkylene, or substituted or unsubstituted heteroalkylene.

L² is a

bond, —NH—, —O—, —S—, —C(O)—, —C(O)NH—, —NHC(O)—, —NHC(O)NH—, —C(O)O—, —OC(O)—, substituted or unsubstituted alkylene, or substituted or unsubstituted heteroalkylene.

In embodiments, R² is —CN,

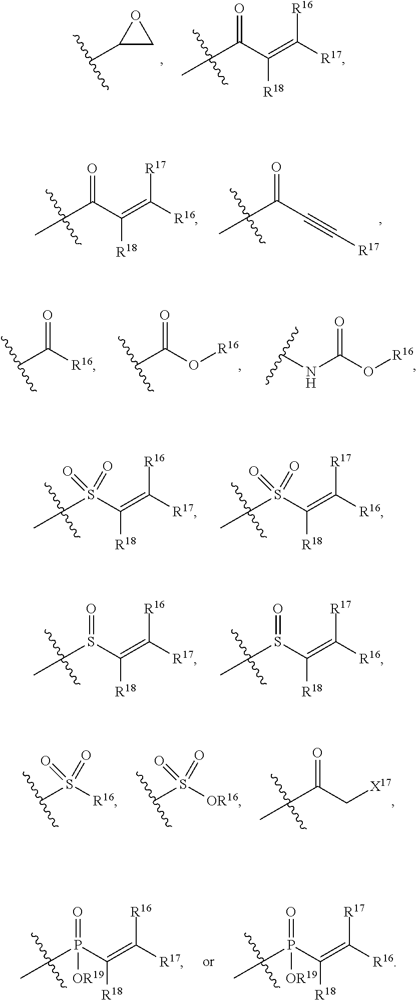

R¹⁶ is hydrogen,

halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC(O)NHNR^{16A}R^{16B},
—NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16B}, —NR^{16A}C(O)R^{16C}, —NR^{16A}C(O)OR^{16C}, —NR^{16A}OR^{16D}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁷ is hydrogen,

halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC(O)NHNR^{17A}R^{17B},
—NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17B}, —NR^{17A}C(O)R^{17C}, —NR^{17A}C(O)OR^{17C}, —NR^{7A}OR^{17D}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁸ is hydrogen,

halogen, —CX¹⁸₃, —CHX¹⁸₂, —CH₂X¹⁸, —CN, —SOₙ₁₈R^{18D}, —SOᵥ₁₈NR^{18A}R^{18B}, —NHNR^{18A}R^{18B}, —ONR^{18A}R^{18B}, —NHC(O)NHNR^{18A}R^{18B},
—NHC(O)NR^{18A}R^{18B}, —N(O)ₘ₁₈, —NR^{1A}R^{18B}, —C(O)R^{18C}, —C(O)—OR^{18C}, —C(O)NR^{18A}R^{18B}, —OR^{18D}, —NR^{18A}SO₂R^{18B}, —NR^{18A}C(O)R^{18C}, —NR^{1A}C(O)OR^{1C}, —NR^{18A}OR^{18D}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁹ is hydrogen,

halogen, —CX¹⁹₃, —CHX¹⁹₂, —CH₂X¹⁹, —CN, —SOₙ₁₉R^{19D}, —SOᵥ₁₉NR^{19A}R^{19B}, —NHNR^{19A}R^{19B}, —ONR^{19A}R^{19B}, —NHC(O)NHNR^{19A}R^{19B},
—NHC(O)NR^{19A}R^{19B}, —N(O)ₘ₁₉, —NR^{19A}R^{19B}, —C(O)R^{19C}, —C(O)—OR^{19C}, —C(O)NR^{19A}R^{19B}, —OR^{19D}, —NR^{19A}SO₂R^{19B}, —NR^{19A}C(O)R^{19C}, —NR^{19A}C(O)OR^{19C}, —R^{19A}OR^{19D}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently

hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl.

X¹⁶, X¹⁷, X¹⁸, and X¹⁹ are independently —F, —Cl, —Br, or —I.

n16, n17, n18, and n19 are independently an integer from 0 to 4.

m16, m17, m18, m19, v16, v17, v18, and v19 are independently 1 or 2.

In an aspect is provided a pharmaceutical composition comprising the compound as described herein and a pharmaceutically acceptable excipient.

In an aspect is provided a method of inhibiting Gαs protein activity, said method comprising: contacting the Gαs protein with a compound as described herein.

In an aspect is provided a method of treating cancer, said method including administering to a subject in need thereof an effective amount of a compound as described herein.

In embodiments, the cancer is pancreatic cancer, a pituitary tumor, or a bone tumor. The cancer is sensitive to Gαs inhibition.

In an aspect is provided a method of treating a bone condition, said method including administering to a subject in need thereof an effective amount of a compound as described herein. In embodiments, the bone condition is fibrous dysplasia. In embodiments, the fibrous dysplasia is monostotic fibrous dysplasia or polystotic fibrous dysplasia.

In an aspect is provided a method of treating McCune-Albright Syndrome, said method including administering to a subject in need thereof an effective amount of a compound described herein.

In an aspect is provided a Gαs protein covalently bonded to a compound as described herein. Gαs is in the GTP state. In embodiments, Gαs is in the GDP state. In embodiments, the compound is bonded to a cysteine residue of the protein.

In embodiments, the Gαs protein has the structure:

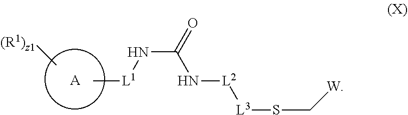

W together with the —CH₂S— to which it is attached form said Gαs protein covalently bonded to a compound.

L³ is substituted or unsubstituted alkylene or substituted or unsubstituted heteroalkylene.

In embodiments, L³ is

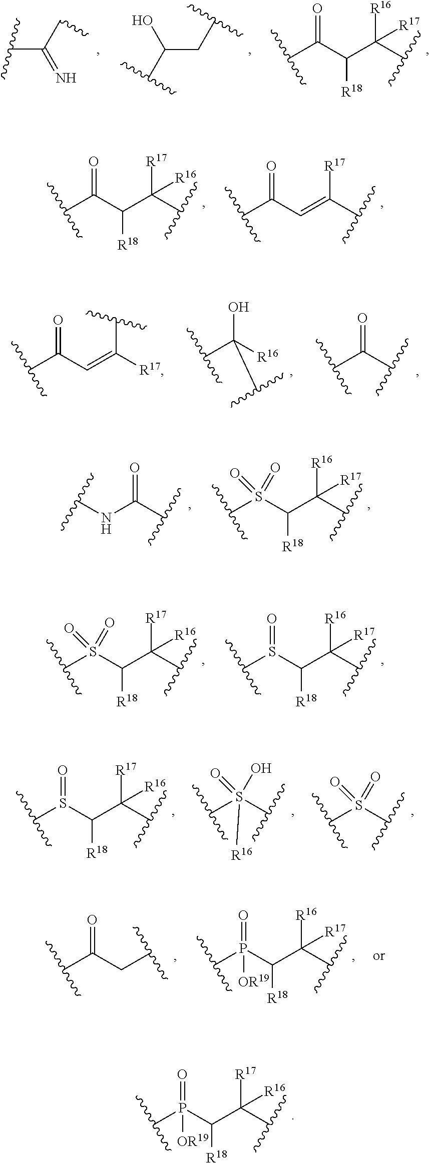

In embodiments, the compound is bonded to cysteine 201. In embodiments, the compound is bonded to cysteine 237.

In an aspect is provided a Gαs protein covalently bonded to a portion of a compound of as described herein.

In an aspect is provided a Gαs protein covalently bonded to a Gαs small molecule inhibitor at R201C. In embodiments, the Gαs protein is a GTP-bound Gαs protein. In embodiments, the Gαs protein is a GDP-bound Gαs protein.

In an aspect is provided a A Gαs protein covalently bonded to a Gαs small molecule inhibitor at C237.

In embodiments, the Gαs protein is a GTP-bound Gαs protein. In embodiments, the Gαs protein is a GDP-bound Gαs protein.

In an aspect is provided a method of treating cancer including administering a Gαs cysteine 201 covalent inhibitor. The Gαs cysteine 201 covalent inhibitor is a compound as described herein.

In an aspect is provided a method of treating cancer including administering a Gαs cysteine 237 covalent inhibitor. The Gαs cysteine 237 covalent inhibitor is a compound as described herein.

Other aspects are disclosed infra.

### BRIEF DESCRIPTION OF THE DRAWINGS

FIG. 1A shows that crystal structure of GNAS(R201C) GDP shows Cys 201, switch-I, switch-II, and switch-III (PDB: 6AU6).

FIGS. 1B-1D show alignment of crystal structure of Gαq-GDP-YM254890 (PDB code 3AH8) with that of Gαs-GTPγS (PDB code 1AZT). FIG. 1B shows YM254890 bound into a pocket surrounded by switch I and the α-helix and β-sheet linked by switch I. FIG. 1C shows that crystal structure of Gαs-GTPγS complex has a very similar domain-arrangement to that of Gαq-GDP-YM254890 complex. FIG. 1D shows comparison with the local structure of YM254890-binding pocket in Gαq reveals a similar binding pocket in Gαs.

FIGS. 1E-1G show structural information for design of a linker to connect YM254890 or its analogues with the cysteine mutation. FIG. 1E shows that the electrostatic surface of Gαq reveals a grove between the helical domain and Ras-like domain (PDB code 3AH8). FIG. 1F shows that local structure of YM254890-binding pocket of Gαq and the distance between R183 and YM254890. FIG. 1G shows that YM254890 is aligned at where the potential inhibitor-binding pocket in Gαs. The hydrophilic residues in the grove between the helical domain and Ras-like domain are showed as sticks.

FIG. 2 shows evaluation of the GTP occupancy of the R201C mutant in the presence of excess GTP.

FIGS. 3A-3C show modification of C201 by acrylamide. FIG. 3A shows that the reaction between the cysteine side chain and acrylamidine (Acr). FIG. 3B shows that modification of C201 by Acr decreased the adenylyl cyclase-activating activity of the GDP-bound R201C/C237S mutant in the presence Gβ1/γ2(C68S). The data represents the mean SE of three independent measurements. FIG. 3C shows that modification of C201 by Acr increased the single turnover GTPase activity of the R201C/C237S mutant to a level close to that of the C237S mutant. The data represents the mean±SD of at least three independent measurements.

FIG. 4A shows a class of compounds with a urea moiety were identified in a tethering screen, in which 2 μM GDP-bound Gαs(R201C/C237S) was incubated with 200 μM tethering compound and 200 μM β-mercaptoethanol (BME) at room temperature for 2 hours.

FIG. 4B shows evaluation of the reactivity of compound 1H11 against Gαs using a competition assay. 2 μM Wild-type or the R201C/C237S mutant Gαs in the GDP or GNP (5′-Guanylyl imidodiphosphate)-bound state was incubated with 50 μM compound 1H11 and various concentration of BME at room temperature for 2 hours.

FIGS. 5A-5B show preliminary structure-activity relationship (SAR) analysis of 1H11. Covalent molecules from the tethering library (FIG. 5A) were tested for their ability to label different Gαs mutants (FIG. 5B).

FIGS. 6A-6B show preliminary structure-activity relationship (SAR) analysis of 1H11. Covalent molecules containing an aryl urea moiety (FIG. 6A) were tested for their BME50 values (FIG. 6B).

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

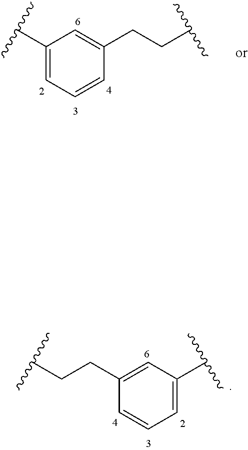 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R′, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂C₁, —CH₂Br, —CH₂F, —CH₂I, —CN, —OH, —NH₂, —C(O)OH, —C(O)NH₂, —NO₂, —SH, —S O₃H, —SO₄H, —SO₂NH₂, —NHNH₂, —ONH₂, —NHC(O)NHNH₂, —NHC(O)NH₂, —NHSO₂H,
—NHC(O)H, —NHC(O)OH, —NHOH, —OCCl₃, —OCF₃, —OCBr₃, —OCI₃, —OCHCl₂, —OCH Br₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, CHCl₂, —CHBr₂, —CHF₂, —CHI₂, —CH₂C₁, —CH₂Br, —CH₂F, —CH₂I, —CN, —OH, —NH₂, —C(O)OH, —C(O)NH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OC HBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, CHCl₂, —CHBr₂, —CHF₂, —CHI₂, —CH₂C₁, —CH₂Br, —CH₂F, —CH₂I, —CN, —OH, —NH₂, —C(O)OH, —C(O)NH₂, —NO₂, —SH,
      —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —O CHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, CHCl₂, —CHBr₂, —CHF₂, —CHI₂, —CH₂C₁, —CH₂Br, —CH₂F, —CH₂I, —CN, —OH, —NH₂, —C(O)OH, —C(O)NH₂, —NO₂, —SH, —SO₃H,
      —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OC HBr₂,
      —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or“lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²¹I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby including another embodiment, and the Markush group is not to be read as a single unit.

As used herein, the terms “bioconjugate” and “bioconjugate reactive moiety” refers to the resulting association between atoms or molecules of bioconjugate reactive groups. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., sulthydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulthydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

“Oxidizing agent” is used in accordance with its ordinary plain meaning within chemistry and biology and refers to a substance that has the ability to oxidize other substances (i.e. removes electrons from the substance). The term “oxidizing agent” is a substance that, in the course of a chemical redox reaction, removes one or more electrons from a substance (e.g., the reactant), wherein the oxidizing agent gains one or more electrons from the substrate. In embodiments, an oxidizing agent is a chemical species that transfers electronegative atoms to another substrate (e.g., a reactant). In embodiments, the oxidizing agent is analogous to the term “electron acceptor” and may be used herein interchangeably. Non-limiting examples of oxidizing agents include oxygen (O₂), ozone (O₃), hydrogen peroxide (H₂O₂), nitric acid (HNO₃), sulfuric acid (H₂SO₄), hexavalent chromium, pyridinium chlorochromate (PCC), N-methylmorpholine-N-oxide (NMO), chromium trioxide (CrO₃, Jones reagent), potassium permanganate (K₂MnO₄), potassium nitrate (KNO₃), Dess-Martin periodinane (DMP), 2-iodoxybenzoic acid (IBX), 2,2,6,6-tetramethylpiperidinyloxy (TEMPO), and Selectfluor® (F-TEDA-BF₄, chloromethyl-4-fluoro-1,4-diazoniabicyclo[2.2.2]octane bis(tetrafluoroborate), potassium perchlorate, or ammonium persulfate.

The term “halogenating agent” is used in accordance with its ordinary plain meaning within chemistry and refers to a substance (e.g., compound or composition) that has the ability to incorporate one or more halogen atoms (e.g. bromination, dibromination tribromination, chlorination, dichlorination, trichlorination, iodination, diiodination, triiodination, fluorination, difluorination, trifluorination, etc.) into another substance (e.g., compound or composition). Halogenating agents include chlorinating agents, brominating agents, iodinating agents and fluorinating agents, wherein a chlorinating agent incorporates a chlorine atom, a brominating agent incorporates a bromine atom, an iodinating agent incorporates an iodine atom, or a fluorinating agent incorporates a fluorine atom. Brominating agents include, but are not limited to, N-bromosuccinimide (NBS), dibromoisocyanuric acid (DBI), bromine, bromotrichloromethane, 1,2-dibromo-1,1,2,2-tetrachloroethane, carbon tetrabromide, tetrabutylammonium tribromide, trimethylphenylammonium tribromide, benzyltrimethylammonium tribromide, pyridinium bromide perbromide, 4-dimethylaminopyridinium bromide perbromide, 1-butyl-3-methylimidazolium tribromide, 1,8-diazabicyclo[5.4.0]-7-undecene, hydrogen tribromide, N-bromophthalimide, N-bromosaccharin, N-bromoacetamide, 2-bromo-2-cyano-N,N-dimethylacetamide, 1,3-dibromo-5,5-dimethylhydantoin, monosodium bromoisocyanurate hydrate, boron tribromide, phosphorus tribromide, bromodimethylsulfonium bromide, 5,5-dibromomeldrum's acid, 2,4,4,6-tetrabromo-2,5-cyclohexadienone, or bis(2,4,6-trimethylpyridine)-bromonium hexafluorophosphate. Chlorinating agents include, but are not limited to, N-chlorosuccinimide (NCS), thionyl chloride, methanesulfonyl chloride, trichloromethanesulfonyl chloride, tert-butyl hypochlorite, chloromethyl methyl ether, dichloromethyl methyl ether, methoxyacetyl chloride, oxalyl chloride, cyanuric chloride, N-chlorophthalimide, sodium dichloroisocyanurate, trichloroisocyanuric acid, chloramine B hydrate, o-chloramine T dihydrate, chloramine T trihydrate, dichloramine B, dichloramine T, benzyltrimethylammonium, tetrachloroiodate. Iodinating agents include, but are not limited to, N-iodosuccinimide (NIS), 1,3-diodo-5,5′-dimethylhidantoin (DIH), iodine, hydriodic acid, diiodomethane, 1-chloro-2-iodoethane, carbon tetraiodide, tetramethylammonium dichloroiodate, benzyltrimethylammonium dichloroiodate, pyridine iodine monochloride, N,N-dimethyl-N-(methylsulfanylmethylene)-ammonium iodide, N-iodosaccharin, trimethylsilyl iodide, bis(pyridine)iodonium tetrafluoroborate, bis(2,4,6-trimethylpyridine)-iodonium hexafluorophosphate. In embodiments, the halogenating agent is not a fluorinating agent.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from their respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving group is designed to facilitate the reaction.

The term “protecting group” is used in accordance with its ordinary meaning in organic chemistry and refers to a moiety covalently bound to a heteroatom to prevent reactivity of the heteroatom during one or more chemical reactions performed prior to removal of the protecting group. In embodiments, the protecting group is covalently bound to a heteroatom that is part of a heteroalkyl, heterocycloalkyl or heteroaryl moiety. Typically a protecting group is bound to a heteroatom (e.g., O) during a part of a multistep synthesis wherein it is not desired to have the heteroatom react (e.g., a chemical reduction) with a reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS), tert-butyl dimethylsilyl (TBS)). In embodiments the protecting group is an amine protecting group. Non-limiting examples of amine protecting groups include carbobenzyloxy (Cbz), p-methoxybenzyl carbonyl (Moz or MeOZ), tert-butyloxycarbonyl (BOC), 9-fluorenylmethyloxycarbonyl (FMOC), acetyl (Ac), benzoyl (Bz), benzyl (Bn), carbamate, p-methoxybenzyl ether (PMB), 3,4-dimethoxybenzyl (DMPM), p-methoxyphenyl (PMP), pivaloyl (Piv), tosyl (Ts), and phthalimide.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 25 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The term “coupling reagent” is used in accordance with its plain ordinary meaning in the arts and refers to a substance (e.g., a compound or solution) which participates in chemical reaction and results in the formation of a covalent bond (e.g., between bioconjugate reactive moieties, between a bioconjugate reactive moiety and the coupling reagent). In embodiments, the level of reagent is depleted in the course of a chemical reaction. This is in contrast to a solvent, which typically does not get consumed over the course of the chemical reaction. Non-limiting examples of coupling reagents include benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), 6-Chloro-benzotriazole-1-yloxy-tris-pyrrolidinophosphonium hexafluorophosphate (PyClock), 1-[Bis(dimethylamino)methylene]-1H-1,2,3-triazolo[4,5-b]pyridinium 3-oxid hexafluorophosphate (HATU), or 2-(1H-benzotriazol-1-yl)-1,1,3,3-tetramethyluronium hexafluorophosphate (HBTU).

The term “solution” is used in accor and refers to a liquid mixture in which the minor component (e.g., a solute or compound) is uniformly distributed within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

A “synergistic amount” as used herein refers to the sum of a first amount (e.g., an amount of a compound provided herein) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the compound provided herein when used separately from the therapeutic agent. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the therapeutic agent when used separately from the compound provided herein.

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., GNAS). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

The term “non-nucleophilic base” as used herein refers to any sterically hindered base that is a poor nucleophile.

The term “nucleophile” as used herein refers to a chemical species that donates an electron pair to an electrophile to form a chemical bond in relation to a reaction. All molecules or ions with a free pair of electrons or at least one pi bond can act as nucleophiles.

The term “strong acid” as used herein refers to an acid that is completely dissociated or ionized in an aqueous solution. Examples of common strong acids include hydrochloric acid (HCl), nitric acid (HNO₃), sulfuric acid (H₂SO₄), hydrobromic acid (HBr), hydroiodic acid (HI), perchloric acid (HClO₄), or chloric acid (HClO₃).

The term “carbocation stabilizing solvent” as used herein refers to any polar protic solvent capable of forming dipole-dipole interactions with a carbocation, thereby stabilizing the carbocation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5-end). An amino acid residue in a protein “corresponds” to a given residue when it occupies the same essential structural position within the protein as the given residue. Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

An “inhibitor” refers to a compound (e.g. compounds described herein) that reduces activity when compared to a control, such as absence of the compound or a compound with known inactivity.

The term “Gαs small molecule inhibitor” as used herein refers to a low molecular weight organic compound capable of binding to and decreasing the activity of Gαs. In embodiments, the Gαs small molecule inhibitor is a compound that weighs less than 1000 daltons. In embodiments, the Gαs small molecule inhibitor is a compound that weighs less than 900 daltons. In embodiments, the Gαs small molecule inhibitor is a compound that weighs less than 800 daltons. In embodiments, the Gαs small molecule inhibitor is a compound that weighs less than 700 daltons. In embodiments, the Gαs small molecule inhibitor is a compound that weighs less than 600 daltons. In embodiments, the Gαs small molecule inhibitor is a compound that weighs less than 500 daltons. In embodiments, the Gαs small molecule inhibitor is a compound that weighs less than 450 daltons. In embodiments, the Gαs small molecule inhibitor is a compound that weighs less than 400 daltons.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The “Gαs cysteine 201 covalent inhibitor” as used herein refers to a compound (e.g., small molecule, antibody, peptide, therapeutic agent, polymer, or the like) which can form a covalent bond with cysteine 201 residue of mutant Gαs protein (R201C mutant of human Gαs protein (SEQ ID NO: 1), or mutants thereof) or a cysteine residue corresponding to cysteine 201 (e.g. in a homologous Gαs mutant protein). In particular, the compound (e.g., compound of Formula (I), (II), (III-a), (III-b), (III-c), (IV), (V-a), (V-b), (VI), (VII), or (VIII)), by forming a covalent bond with the —SH group of that cysteine, can inhibit, suppress or downregulate the function of the Gαs protein (e.g., human Gαs, or R201C mutant of human Gαs protein (SEQ ID NO: 1)).

The “Gαs cysteine 237 covalent inhibitor” as used herein refers to a compound (e.g., small molecule, antibody, peptide, therapeutic agent, polymer, or the like) which can form a covalent bond with cysteine 237 residue of Gαs protein (e.g., human Gαs, protein represented by SEQ ID NO: 1, or mutants thereof) or a cysteine residue corresponding to cysteine 237 (e.g. in a homologous Gαs protein). In particular, the compound (e.g., compound of Formula (I), (II), (III-a), (III-b), (III-c), (IV), (V-a), (V-b), (VI), (VII), or (VIII)), by forming a covalent bond with the —SH group of that cysteine, can inhibit, suppress or downregulate the function of the Gαs protein (e.g., human Gαs, protein represented by SEQ ID NO: 1, or mutants thereof).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “therapeutic agent” or “drug agent” as used herein refers to an agent (e.g., compound or composition) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being. A drug moiety is a monovalent drug. A therapeutic moiety is a monovalent therapeutic agent.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

“G protein associated cancer” (also referred to herein as “G-protein related cancer”) refers to a cancer caused by aberrant activity or signaling of G protein or one or more of its subunits (e.g., alpha (α)-, beta (β)-, or gamma (γ) subunits; Gαs, Gβs, or Gγs). In certain embodiments, a “cancer associated with aberrant Gαs activity” (also referred to herein as “Gαs related cancer”) is a cancer caused by aberrant Gαs activity or signaling (e.g. a mutant Gαs). In certain embodiments, a “cancer associated with aberrant Gβs activity” (also referred to herein as “Gβs related cancer”) is a cancer caused by aberrant Gβs activity or signaling (e.g. a mutant Gβs). In certain embodiments, a “cancer associated with aberrant Gγs activity” (also referred to herein as “Gγs related cancer”) is a cancer caused by aberrant Gγs activity or signaling (e.g. a mutant Gγs). In certain embodiments, some cancers that are associated with aberrant activity of one or more of G protein or its subunits (Gαs, Gβs, or Gγs), mutant G protein, or mutants subunits (Gαs, Gβs, or Gγs) are well known in the art and determining such cancers are within the skill of a person of skill in the art. In certain embodiments, some cancers may be sensitive to Gαs inhibition. In certain embodiments, the cancer that may be sensitive to Gαs inhibition may include a solid cancer or a tumor. In certain embodiments, the cancer that may be sensitive to Gαs inhibition may include a pancreatic cancer, a brain tumor, a pituitary tumor, or a bone tumor. In certain embodiments, the Gαs related cancers may include a pancreatic cancer, a brain tumor, a pituitary tumor, or a bone tumor.

“G protein-associated disease” (also referred to herein as “G protein-related disease”) refers to a cancer caused by aberrant activity or signaling of G protein or one or more of its subunits (e.g., alpha (α)-, beta (β)-, or gamma (γ) subunits; Gαs, Gβs, or Gγs). In certain embodiments, a “disease associated with aberrant Gαs activity” (also referred to herein as “Gαs related disease”) is a cancer caused by aberrant Gαs activity or signaling (e.g., a mutant Gαs). In certain embodiments, a “disease associated with aberrant Gβs activity” (also referred to herein as “Gβs related disease”) is a disease caused by aberrant Gβs activity or signaling (e.g., a mutant Gβs). In certain embodiments, a “disease associated with aberrant Gγs activity” (also referred to herein as “Gγs related disease”) is a disease caused by aberrant Gγs activity or signaling (e.g., a mutant Gγs). In certain embodiments, some diseases that are associated with aberrant activity of one or more of G protein or its subunits (Gαs, Gβs, or Gγs), mutant G protein, or mutants subunits (Gαs, Gβs, or Gγs) are well known in the art and determining such diseases are within the skill of a person of skill in the art. In certain embodiments, some diseases may be sensitive to Gαs inhibition.

The term “guanine nucleotide-binding proteins” or “G-protein” refers to one or more of the family of proteins that are bound to GTP (“on” state) or GDP (“off” state”) so the proteins can regulate their activity involved in signaling pathway of a cell. In certain embodiments, G protein includes subunits, alpha (α)-, beta (β)-, and gamma (γ) subunits (Gαs, Gβs, or Gγs). In particular, the term human “Gαs” as used herein refers to a G-protein-alpha-subunit having nucleotide sequences as set forth or corresponding to Entrez 2778, UniProt Q59FM5, UniProt P63092 (e.g., UniProt P6309-1 and UniProt P63092-2), RefSeq (protein) NP_000507.1, RefSeq (protein) NP_001070956.1, RefSeq (protein) NP_001070957.1, RefSeq (protein) NP_001070958.1, RefSeq (protein) NP_001296769.1, RefSeq (protein) NP 536350.2, or RefSeq (protein) NP_536351.1. In embodiments, the GNAS gene has the nucleic acid sequence set forth in RefSeq (mRNA) NM_000516.5, RefSeq (mRNA) NM_001077488.3, RefSeq (mRNA) NM_001077489.3, RefSeq (mRNA) NM_001077490.2, RefSeq (mRNA) NM 001309840.1, RefSeq (mRNA) NM_080425.3, or RefSeq (mRNA) NM_080426.3. In embodiments, the amino acid sequence or nucleic acid sequence is the sequence known at the time of filing of the present application.

The term “Gαs” includes both the wild-type form of the nucleotide sequences or proteins as well as any mutants thereof. In certain embodiments, the human Gαs refers to the protein including (e.g., consisting of) the amino acid sequence corresponding to UniProt P63092-1 (SEQ ID NO: 1). In embodiments, the human Gαs includes the sequence below with one or more mutations (e.g., R201C and C237S at the underlined position at SEQ ID NO: 1):

| (SEQ ID NO: 1) | |
|---|---|
| 1 | MGCLGNSKTE DQRNEEKAQR EANKKIEKQL QKDKQVYRAT HRLLLLGAGE SGKSTIVKQM |
| 61 | RILHVNGFNG EGGEEDPQAA RSNSDGEKAT KVQDIKNNLK EAIETIVAAM SNLVPPVELA |
| 121 | NPENQFRVDY ILSVMNVPDF DFPPEFYEHA KALWEDEGVR ACYERSNEYQ LIDCAQYFLD |
| 181 | KIDVIKQADY VPSDQDLLRC RVLTSGIFET KFQVDKVNFH MFDVGGQRDE RRKWIQCFND |
| 241 | VTAIIFVVAS SSYNMVIRED NQTNRLQEAL NLFKSIWNNR WLRTISVILF LNKQDLLAEK |
| 301 | VLAGKSKIED YFPEFARYTT PEDATPEPGE DPRVTRAKYF IRDEFLRIST ASGDGRHYCY |
| 361 | PHFTCAVDTE NIRRVFNDCR DIIQRMHLRQ YELL |

In embodiments, the human Gαs has the sequence of residues 7-380 of the short isoform of human Gαs corresponding to UniProt P63092-2 (SEQ ID NO: 2). In embodiments, the human Gαs includes the sequence below with one or more mutations (e.g., at R187 and/or C223 at the underlined position at SEQ ID NO: 2)

(SEQ ID NO: 2)
HMGCLGNSKTEDQRNEEKAQREANKKIEKQLQKDKQVYRATHRLLLLGA
GESGKSTIVKQMRILHVNGFNGDSEKATKVQDIKNNLKEAIETIVAAMS
NLVPPVELANPENQFRVDYILSVMNVPDFDFPPEFYEHAKALWEDEGVR
ACYERSNEYQLIDCAQYFLDKIDVIKQADYVPSDQDLLRCRVLTSGIFE
TKFQVDKVNFHMFDVGGQRDERRKWIQCFNDVTAIIFVVASSSYNMVIR
EDNQTNRLQEALNLFKSIWNNRWLRTISVILFLNKQDLLAEKVLAGKSK
IEDYFPEFARYTTPEDATPEPGEDPRVTRAKYFIRDEFLRISTASGDGR
HYCYPHFTCAVDTENIRRVFNDCRDIIQRMHLRQYELL

An amino acid residue in Gαs “corresponds” to a given residue when it occupies the same essential structural position within the protein as the given residue. For example, a selected residue in a selected protein corresponds to R201 of Gαs protein when the selected residue occupies the same essential spatial or other structural relationship as R201 of Gαs protein. In some embodiments, where a selected protein is aligned for maximum homology with the Gαs protein, the position in the aligned selected protein aligning with R201 is said to correspond to R201. Further, a selected residue in a selected protein corresponds to C237 of Gαs protein when the selected residue occupies the same essential spatial or other structural relationship as C237 of Gαs protein. In some embodiments, where a selected protein is aligned for maximum homology with the Gαs protein, the position in the aligned selected protein aligning with C237 is said to correspond to C237. Instead of a primary sequence alignment, a three dimensional structural alignment can also be used, e.g., where the structure of the selected protein is aligned for maximum correspondence with the Gαs protein and the overall structures compared. In this case, an amino acid that occupies the same essential position as R201 in the structural model is said to correspond to the R201 residue, and an amino acid that occupies the same essential position as C237 in the structural model is said to correspond to the C237 residue. For example, R201 in SEQ ID NO: 1 corresponds to R187 in SEQ ID NO: 2, and C237 in SEQ ID NO: 1 corresponds to C223 in SEQ ID NO: 2.

The term “bone condition” as used herein refers to a disease, disorder or condition caused by abnormal bone tissues (e.g., osteoblast, osteoclast, osteocyte, and hematopoietic). In embodiments, the bone condition is caused by, but not limited to, cancerous or noncancerous tissues, infection, osteoporosis, tumor, blood cells, and fibrous tissues, which is developed in various sites of bones of a subject such as thighbone, skull, ribs, pelvis, humerus, shinbone, trunk, sternum, wrist bones, tarsals, spine, shoulder blade, collar bone, radius, ulna, metacarpals, phalanges, kneecap, fibula, metatarsals and phalanges. In certain embodiments, the bone condition may be caused by cancerous bone tissues or noncancerous bone tissues. In certain embodiments, the bone condition may be related to abnormal fibrous tissue development/occurrence in place of normal bone.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The term “administer (or administering) a Gαs inhibitor” means administering a compound that decreases the activity or level (e.g. amount) of a signaling pathway of Gαs to a subject. Administration may include, without being limited by mechanism, allowing sufficient time for the Gαs inhibitor to reduce the activity of the Gαs protein or for the Gαs inhibitor to reduce one or more symptoms of a disease (e.g. cancer, wherein the Gαs inhibitor may arrest the cell cycle, slow the cell cycle, reduce DNA replication, reduce cell replication, reduce cell growth, reduce metastasis, or cause cell death). In embodiments, the administering does not include administration of any active agent (e.g., a compound or Gαs inhibitor) other than the recited active agent.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “electrophilic chemical moiety” is used in accordance with its plain ordinary chemical meaning and refers to a monovalent chemical group that is electrophilic.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “sensitive” or “sensitive to” as used herein refers to a high degree of change in substance activity, biomarker indication, or condition associated with a disease (e.g. cancer) or a symptom of the disease (e.g., cancer) in response to a change introduced by treatment with or contact to an agent. For example, by treating with or contacting with an agent (e.g., Gαs inhibitor or Gαs mutant inhibitor), the substance activity, biomarker indication, or condition associated with a disease (e.g. cancer) or a symptom of the disease (e.g., cancer) varies substantially compared to those in absence of any treatment or contacting to the agent. In some embodiments, a disease (e.g. cancer) may be sensitive to a causative agent or inhibitory agent that may cause the disease. In some embodiments, a cancer relevant to or associated with aberrant protein activity (e.g., increased/suppressed protein activity or function) or mutation thereof may be sensitive to the inhibition of the protein. In some embodiments, a cancer caused or developed in association with aberrant Gαs activity (e.g., increased/suppressed Gαs activity or function) or mutation thereof may be sensitive to the inhibition of the Gαs or its mutants.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol™ (i.e., paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39·HCl), AC-7700 (Ajinomoto, i.e., AVE-8062, AVE-8062A, CS-39-L-Ser·HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/IKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid) used to treat cancer through destruction or inhibition of cancer cells or tissues. Anticancer agents may be selective for certain cancers or certain tissues. In embodiments, anticancer agents herein may include epigenetic inhibitors and single- or multi-kinase inhibitors (e.g., G-protein inhibitor or Gαs inhibitor).

### II. Compounds

Provided herein, inter alia, are compounds. The compounds may be state-selective Gαs labeling molecules, for example, based on disulfide tethering. The compounds may label the somatic cysteine mutant selectively over all other cysteines present in the Gαs protein, for example, forming a covalent irreversible bonding to the protein.

In an aspect provided is a compound having the formula:

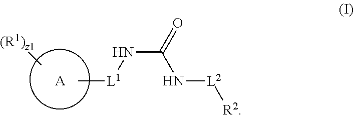

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C (O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

The variable z1 is an integer from 0 to 6.

Ring A is aryl or heteroaryl.

L¹ is a

bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L² is a

bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R² is an electrophilic moiety.

R^{1A}, R^{1B}, R^{1C}, and R^{1D} are independently

hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl.

X and X¹ are independently —F, —Cl, —Br, or —I.

n1 is independently an integer from 0 to 4.

m1 and v1 are independently 1 or 2.

In embodiments, z1 is an integer from 1 to 3. In embodiments, z1 is 0. In embodiments, z1 is 1. In embodiments, z1 is 2. In embodiments, z1 is 3.

In embodiments, Ring A is phenyl or 5 to 6-membered heteroaryl. In embodiments, Ring A is phenyl. In embodiments, Ring A is 5 to 6-membered heteroaryl. In embodiments, Ring A is 5-membered heteroaryl. In embodiments, Ring A is 6-membered heteroaryl. In embodiments, Ring A is 5-membered heteroaryl containing at least one nitrogen atom. In embodiments, Ring A is 6-membered heteroaryl containing at least one nitrogen atom.

In embodiments, the compound has the formula:

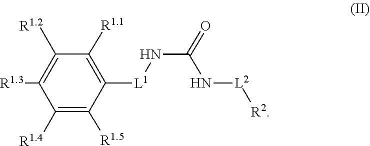

L¹, L², and R² are as described herein.

Each R^{1.1}, R^{1.2}, R^{1.3}, R^{1.4}, and R^{1.5} is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B},

—NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, and R^{1D} are independently

hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl.

X and X¹ are independently —F, —Cl, —Br, or —I.

n1 is independently an integer from 0 to 4.

m1 and v1 are independently 1 or 2.

In embodiments, L¹ is a

bond, —NH—, —O—, —S—, —C(O)—, —C(O)NH—, —NHC(O)—, —NHC(O)NH—, —C(O)O—, —OC(O)—, substituted or unsubstituted alkylene, or substituted or unsubstituted heteroalkylene. In embodiments, L¹ is a
bond, —NH—, —O—, —S—, —C(O)—, —C(O)NH—, —NHC(O)—, —NHC(O)NH—, —C(O)O—, —OC(O)—, substituted or unsubstituted C₁-C₆ alkylene, or substituted or unsubstituted 2 to 6 membered heteroalkylene.

In embodiments, L¹ is a bond. In embodiments, L¹ is —NH—. In embodiments, L¹ is —O—. In embodiments, L¹ is —S—. In embodiments, L¹ is —C(O)—. In embodiments, L¹ is —C(O)NH—. In embodiments, L¹ is —NHC(O)—. In embodiments, L¹ is —NHC(O)NH—. In embodiments, L¹ is —C(O)O—. In embodiments, L¹ is —OC(O)—.

In embodiments, L¹ is substituted or unsubstituted C₁-C₆ alkylene. In embodiments, L¹ is substituted C₁-C₆ alkylene. In embodiments, L¹ is unsubstituted C₁-C₆ alkylene. In embodiments, L¹ is substituted or unsubstituted C₁-C₄ alkylene. In embodiments, L¹ is substituted C₁-C₄ alkylene. In embodiments, L¹ is unsubstituted C₁-C₄ alkylene. In embodiments, L¹ is substituted or unsubstituted C₁-C₃ alkylene. In embodiments, L¹ is substituted C₁-C₃ alkylene. In embodiments, L¹ is unsubstituted C₁-C₃ alkylene. In embodiments, L¹ is substituted or unsubstituted methylene. In embodiments, L¹ is substituted methylene. In embodiments, L¹ is unsubstituted methylene. In embodiments, L¹ is substituted or unsubstituted ethylene. In embodiments, L¹ is substituted ethylene. In embodiments, L¹ is unsubstituted ethylene.

In embodiments, L¹ is substituted or unsubstituted 2 to 6 membered heteroalkylene. In embodiments, L¹ is substituted 2 to 6 membered heteroalkylene. In embodiments, L¹ is unsubstituted 2 to 6 membered heteroalkylene. In embodiments, L¹ is substituted or unsubstituted 2 to 5 membered heteroalkylene. In embodiments, L¹ is substituted 2 to 5 membered heteroalkylene. In embodiments, L¹ is unsubstituted 2 to 5 membered heteroalkylene. In embodiments, L¹ is substituted or unsubstituted 2 to 4 membered heteroalkylene. In embodiments, L¹ is substituted 2 to 4 membered heteroalkylene. In embodiments, L¹ is unsubstituted 2 to 4 membered heteroalkylene. In embodiments, L¹ is substituted or unsubstituted 2 to 3 membered heteroalkylene. In embodiments, L¹ is substituted 2 to 3 membered heteroalkylene. In embodiments, L¹ is unsubstituted 2 to 3 membered heteroalkylene.

In embodiments, L² is an unsubstituted C₁-C₆ alkylene. In embodiments, L² is an unsubstituted C₁-C₅ alkylene. In embodiments, L² is an unsubstituted C₁-C₄ alkylene. In embodiments, L² is an unsubstituted C₁-C₃ alkylene. In embodiments, L² is an unsubstituted C₁-C₂ alkylene. In embodiments, L² is unsubstituted methylene. In embodiments, L² is unsubstituted ethylene. In embodiments, L² is unsubstituted propylene. In embodiments, L² is unsubstituted isopropylene. In embodiments, L² is unsubstituted butylene. In embodiments, L² is unsubstituted isobutylene. In embodiments, L² is unsubstituted t-butylene. In embodiments, L² is unsubstituted 2-methyl propylene. In embodiments, L² is a bond.

In embodiments, R^{1.1} is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1.2} is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}S02R^{1D}, —NR^{1A}C(O)R^{1C}, —NR^{1A}C(O)OR^{1C}, —NR^{1A}OR^{1C}, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1.3} is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}S02R^{1D}, —NR^{1A}C(O)R^{1C}, —NR^{1A}C(O)OR^{1C}, —NR^{1A}OR^{1C}, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1.4} is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}S02R^{1D}, —NR^{1A}C(O)R^{1C}, —NR^{1A}C(O)OR^{1C}, —NR^{1A}OR^{1C}, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1.5} is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}S02R^{1D}, —NR^{1A}C(O)R^{1C}, —NR^{1A}C(O)OR^{1C}, —NR^{1A} OR^{1C}, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, R^{1A} is hydrogen, or substituted or unsubstituted alkyl. In embodiments, R^{1A} is hydrogen. In embodiments, R^{1A} is substituted or unsubstituted alkyl. In embodiments, R^{1A} is substituted alkyl. In embodiments, R^{1A} is unsubstituted alkyl. In embodiments, R^{1A} is substituted or unsubstituted C₁-C₆ alkyl. In embodiments, R^{1A} is unsubstituted C₁-C₆ alkyl. In embodiments, R^{1A} is substituted or unsubstituted C₁-C₅ alkyl. In embodiments, R^{1A} is unsubstituted C₁-C₅ alkyl. In embodiments, R^{1A} is substituted or unsubstituted C₁-C₄ alkyl. In embodiments, R^{1A} is unsubstituted C₁-C₄ alkyl. In embodiments, R^{1A} is methyl. In embodiments, R^{1A} is ethyl. In embodiments, R^{1A} is propyl. In embodiments, R^{1A} is isopropyl. In embodiments, R^{1A} is butyl. In embodiments, R^{1A} is t-butyl.

In embodiments, R^{1B} is hydrogen, or substituted or unsubstituted alkyl. In embodiments, R^{1B} is hydrogen. In embodiments, R^{1B} is substituted or unsubstituted alkyl. In embodiments, R^{1B} is substituted alkyl. In embodiments, R^{1B} is unsubstituted alkyl. In embodiments, R^{1B} is substituted or unsubstituted C₁-C₆ alkyl. In embodiments, R^{1B} is unsubstituted C₁-C₆ alkyl. In embodiments, R^{1B} is substituted or unsubstituted C₁-C₅ alkyl. In embodiments, R^{1B} is unsubstituted C₁-C₅ alkyl. In embodiments, R^{1B} is substituted or unsubstituted C₁-C₄ alkyl. In embodiments, R^{1B} is unsubstituted C₁-C₄ alkyl. In embodiments, R^{1B} is methyl. In embodiments, R^{1B} is ethyl. In embodiments, R^{1B} is propyl. In embodiments, R^{1B} is isopropyl. In embodiments, R^{1B} is butyl. In embodiments, R^{1B} is t-butyl.

In embodiments, R^{1C} is hydrogen, or substituted or unsubstituted alkyl. In embodiments, R^{1C} is hydrogen. In embodiments, R^{1C} is substituted or unsubstituted alkyl. In embodiments, R^{1C} is substituted alkyl. In embodiments, R^{1C} is unsubstituted alkyl. In embodiments, R^{1C} is substituted or unsubstituted C₁-C₆ alkyl. In embodiments, R^{1C} is unsubstituted C₁-C₆ alkyl. In embodiments, R^{1C} is substituted or unsubstituted C₁-C₅ alkyl. In embodiments, R^{1C} is unsubstituted C₁-C₅ alkyl. In embodiments, R^{1C} is substituted or unsubstituted C₁-C₄ alkyl. In embodiments, R^{1C} is unsubstituted C₁-C₄ alkyl. In embodiments, R^{1C} is methyl. In embodiments, R^{1C} is ethyl. In embodiments, R^{1C} is propyl. In embodiments, R^{1C} is isopropyl. In embodiments, R^{1C} is butyl. In embodiments, R^{1C} is t-butyl.

In embodiments, R^{1D} is hydrogen, or substituted or unsubstituted alkyl. In embodiments, R^{1D} is hydrogen. In embodiments, R^{1D} is substituted or unsubstituted alkyl. In embodiments, R^{1D} is substituted alkyl. In embodiments, R^{1D} is unsubstituted alkyl. In embodiments, R^{1D} is substituted or unsubstituted C₁-C₆ alkyl. In embodiments, R^{1D} is unsubstituted C₁-C₆ alkyl. In embodiments, R^{1D} is substituted or unsubstituted C₁-C₅ alkyl. In embodiments, R^{1D} is unsubstituted C₁-C₅ alkyl. In embodiments, R^{1D} is substituted or unsubstituted C₁-C₄ alkyl. In embodiments, R^{1D} is unsubstituted C₁-C₄ alkyl. In embodiments, R^{1D} is methyl. In embodiments, R^{1D} is ethyl. In embodiments, R^{1D} is propyl. In embodiments, R^{1D} is isopropyl. In embodiments, R^{1D} is butyl. In embodiments, R^{1D} is t-butyl.

In embodiments, R^{1.1} is —CN. In embodiments, R^{1.1} is hydrogen.

In embodiments, R^{1.2} is halogen (e.g., —F, —Cl, —Br, or —I) or —CN. In embodiments, In embodiments, R^{1.2} is —F. In embodiments, R^{1.2} is —Cl. In embodiments, R^{1.2} is —Br. In embodiments, R^{1.2} is —I. In embodiments, R^{1.2} is —CN. In embodiments, R^{1.2} is hydrogen.

In embodiments, R^{1.2} is —OR^{1D}. In embodiments, R^{1D} is hydrogen, or substituted or unsubstituted C₁-C₄ alkyl. In embodiments, R^{1D} is hydrogen. In embodiments, R^{1D} is substituted or unsubstituted C₁-C₄ alkyl. In embodiments, R^{1D} is unsubstituted C₁-C₄ alkyl. In embodiments, R^{1D} is methyl. In embodiments, R^{1D} is ethyl. In embodiments, R^{1D} is propyl. In embodiments, R^{1D} is isopropyl. In embodiments, R^{1D} is butyl. In embodiments, R^{1D} is t-butyl. In embodiments, R^{1.2} is —OH. In embodiments, R^{1.2} is —OCH₃. In embodiments, R^{1.2} is —OCH₂CH₃.

R^{1.3} is halogen, —CN, substituted or unsubstituted C₁-C₆ alkyl, or substituted or unsubstituted 2 to 6 membered heteroalkyl. In embodiments, R^{1.3} is —F. In embodiments, R^{1.3} is —Cl. In embodiments, R^{1.3} is —Br. In embodiments, R^{1.3} is —I. In embodiments, R^{1.3} is —CN. In embodiments, R^{1.3} is hydrogen.

In embodiments, R^{1.3} is substituted or unsubstituted C₁-C₆ alkyl. In embodiments, R^{1.3} is substituted C₁-C₆ alkyl. In embodiments, R^{1.3} is unsubstituted C₁-C₆ alkyl. In embodiments, R^{1.3} is substituted or unsubstituted C₁-C₅ alkyl. In embodiments, R^{1.3} is substituted C₁-C₅ alkyl. In embodiments, R^{1.3} is unsubstituted C₁-C₅ alkyl. In embodiments, R^{1.3} is substituted or unsubstituted C₁-C₄ alkyl. In embodiments, R^{1.3} is substituted C₁-C₄ alkyl. In embodiments, R^{1.3} is unsubstituted C₁-C₄ alkyl. In embodiments, R^{1.3} is substituted or unsubstituted C₂-C₄ alkyl. In embodiments, R^{1.3} is substituted C₂-C₄ alkyl. In embodiments, R^{1.3} is unsubstituted C₂-C₄ alkyl. In embodiments, R^{1.3} is substituted or unsubstituted C₁-C₃ alkyl. In embodiments, R^{1.3} is substituted C₁-C₃ alkyl. In embodiments, R^{1.3} is unsubstituted C₁-C₃ alkyl. In embodiments, R^{1.3} is OH-substituted C₁-C₆ alkyl. In embodiments, R^{1.3} is OH-substituted C₁-C₄ alkyl. In embodiments, R^{1.3} is —CH₂OH. In embodiments, R^{1.3} is —CH₂CH₂OH. In embodiments, R^{1.3} is —CH₂CH₂CH₂OH. In embodiments, R^{1.3} is —CH₂CH₂CH₂CH₂OH.

In embodiments, R^{1.3} is substituted or unsubstituted 2 to 6 membered heteroalkyl. In embodiments, R^{1.3} is substituted 2 to 6 membered heteroalkyl. In embodiments, R^{1.3} is unsubstituted 2 to 6 membered heteroalkyl. In embodiments, R^{1.3} is substituted or unsubstituted 2 to 5 membered heteroalkyl. In embodiments, R^{1.3} is substituted 2 to 5 membered heteroalkyl. In embodiments, R^{1.3} is unsubstituted 2 to 5 membered heteroalkyl. In embodiments, R^{1.3} is substituted or unsubstituted 2 to 4 membered heteroalkyl. In embodiments, R^{1.3} is substituted 2 to 4 membered heteroalkyl. In embodiments, R^{1.3} is unsubstituted 2 to 4 membered heteroalkyl. In embodiments, R^{1.3} is substituted or unsubstituted 3 to 6 membered heteroalkyl. In embodiments, R^{1.3} is substituted 3 to 6 membered heteroalkyl. In embodiments, R^{1.3} is unsubstituted 3 to 6 membered heteroalkyl. In embodiments, R^{1.3} is substituted or unsubstituted 3 to 5 membered heteroalkyl. In embodiments, R^{1.3} is substituted 3 to 5 membered heteroalkyl. In embodiments, R^{1.3} is unsubstituted 3 to 5 membered heteroalkyl. In embodiments, R^{1.3} is substituted or unsubstituted 3 to 4 membered heteroalkyl. In embodiments, R^{1.3} is substituted 3 to 4 membered heteroalkyl. In embodiments, R^{1.3} is unsubstituted 3 to 4 membered heteroalkyl. In embodiments, R^{1.3} is —CH₂OH. In embodiments, R^{1.3} is —CH₂CH₂OH. In embodiments, R^{1.3} is —CH₂CH₂CH₂OH. In embodiments, R^{1.3} is —CH₂CH₂CH₂CH₂OH.

In embodiments, R^{1.3} is —C(O)R^{1C}, or —C(O)—OR^{1C}. In embodiments, R^{1.3} is —C(O)R^{1C}. In embodiments, R^{1.3} is —C(O)—OR^{1C}. In embodiments, R^{1C} is hydrogen, or substituted or unsubstituted C₁-C₄ alkyl. In embodiments, R^{1C} is hydrogen. In embodiments, R^{1C} is substituted or unsubstituted C₁-C₄ alkyl. In embodiments, R^{1C} is unsubstituted C₁-C₄ alkyl. In embodiments, R^{1C} is methyl. In embodiments, R^{1C} is ethyl. In embodiments, R^{1C} is propyl. In embodiments, R^{1C} is isopropyl. In embodiments, R^{1C} is butyl. In embodiments, R^{1C} is t-butyl. In embodiments, R^{1.3} is —C(O)H. In embodiments, R^{1.3} is —C(O)CH₃. In embodiments, R^{1.3} is —C(O)CH₂CH₃. In embodiments, R^{1.3} is —C(O)CH₂CH₂CH₃. In embodiments, R^{1.3} is —C(O)CH₂CH₂CH₂CH₃. In embodiments, R^{1.3} is —C(O)OH. In embodiments, R^{1.3} is —C(O)OCH₃. In embodiments, R^{1.3} is —C(O)OCH₂CH₃. In embodiments, R^{1.3} is —C(O)OCH₂CH₂CH₃. In embodiments, R^{1.3} is —C(O)OCH₂CH₂CH₂CH₃.

In embodiments, R^{1.3} is —OR^{1D}. In embodiments, R^{1D} is hydrogen, or substituted or unsubstituted C₁-C₄ alkyl. In embodiments, R^{1D} is hydrogen. In embodiments, R^{1D} is substituted or unsubstituted C₁-C₄ alkyl. In embodiments, R^{1D} is unsubstituted C₁-C₄ alkyl. In embodiments, R^{1D} is methyl. In embodiments, R^{1D} is ethyl. In embodiments, R^{1D} is propyl. In embodiments, R^{1D} is isopropyl. In embodiments, R^{1D} is butyl. In embodiments, R^{1D} is phenyl. In embodiments, R^{1D} is t-butyl. In embodiments, R^{1.3} is —OH. In embodiments, R^{1.3} is —OCH₃. In embodiments, R^{1.3} is —OCH₂CH₃. In embodiments, R^{1.3} is —OC₅H₆.

In embodiments, R^{1.3} is —OCX¹₃, —OCH₂X¹, or —OCHX¹₂. In embodiments, R^{1.3} is —OCX¹₃. In embodiments, R^{1.3} is —OCH₂X¹. In embodiments, R^{1.3} is —OCHX¹₂. In embodiments, R^{1.3} is —OCF₃, —OCCl₃, —OCBr₃, or —OCI₃. In embodiments, R^{1.3} is —OCHF₂, —OCHCl₂, —OCHBr₂, or —OCHI₂. In embodiments, R^{1.3} is —OCH₂F, —OCH₂Cl, —OCH₂Br, or —OCH₂I. In embodiments, R^{1.3} is —OCF₃.

In embodiments, R^{1.4} is halogen (e.g., —F, —Cl, —Br, or —I) or —CN. In embodiments, In embodiments, R^{1.4} is —F. In embodiments, R^{1.4} is —Cl. In embodiments, R^{1.4} is —Br. In embodiments, R^{1.4} is —I. In embodiments, R^{1.4} is —CN. In embodiments, R^{1.4} is hydrogen.

In embodiments, R^{1.4} is —OR^{1D}. In embodiments, R^{1D} is hydrogen, or substituted or unsubstituted C₁-C₄ alkyl. In embodiments, R^{1D} is hydrogen. In embodiments, R^{1D} is substituted or unsubstituted C₁-C₄ alkyl. In embodiments, R^{1D} is unsubstituted C₁-C₄ alkyl. In embodiments, R^{1D} is methyl. In embodiments, R^{1D} is ethyl. In embodiments, R^{1D} is propyl. In embodiments, R^{1D} is isopropyl. In embodiments, R^{1D} is butyl. In embodiments, R^{1D} is t-butyl. In embodiments, R^{1.4} is —OH. In embodiments, R^{1.4} is —OCH₃. In embodiments, R^{1.4} is —OCH₂CH₃.

In embodiments, R^{1.5} is —CN. In embodiments, R^{1.5} is hydrogen.

In embodiments, R^{1.2} and R^{1.4} are hydrogen; and R^{1.3} is substituted or unsubstituted 2 to 6 membered heteroalkyl. In embodiments, R^{1.1}, R^{1.2}, R^{1.4} and R^{1.5} are hydrogen; and R^{1.3} is substituted or unsubstituted 2 to 6 membered heteroalkyl. In embodiments, R^{1.1}, R^{1.2}, R^{1.4} and R^{1.5} is hydrogen; and R^{1.3} is —CH₂OH, —CH₂CH₂OH, —CH₂CH₂CH₂OH, or —CH₂CH₂CH₂CH₂OH. In embodiments, R^{1.1}, R^{1.2}, R^{1.4} and R^{1.5} is hydrogen; and R^{1.3} is —CH₂CH₂OH. In embodiments, R^{1.2} and R^{1.4} is hydrogen; and R^{1.3} is —CN. In embodiments, R^{1.1}, R^{1.2}, R^{1.4} and R^{1.5} is hydrogen; and R^{1.3} is —CN.

In embodiments, R^{1.2} is halogen (e.g., —F, —Cl, —Br, or —I); and R^{1.5} is —CN. In embodiments, R^{1.2} is —Cl, and R^{1.5} is —CN. In embodiments, R^{1.2} is halogen (e.g., —F, —Cl, —Br, or —I); R^{1.3} is hydrogen, and R^{1.5} is —CN. In embodiments, R^{1.2} is —Cl; R^{1.3} is hydrogen; and R^{1.5} is —CN. In embodiments, R^{1.1}, R^{1.3}, and R^{1.4} are hydrogen; R^{1.2} is halogen (e.g., —F, —Cl, —Br, or —I); and R^{1.5} is —CN. In embodiments, R^{1.1}, R^{1.3}, and R^{1.4} are hydrogen; R^{1.2} is —Cl; and R^{1.5} is —CN.

In embodiments, R^{1.1} is —CN; and R^{1.4} is halogen (e.g., —F, —Cl, —Br, or —I). In embodiments, R^{1.1} is —CN; and R^{1.4} is —Cl. In embodiments, R^{1.1} is —CN; R^{1.3} is hydrogen; and R^{1.4} is halogen (e.g., —F, —Cl, —Br, or —I). In embodiments, R^{1.1} is —CN; R^{1.3} is hydrogen; and R^{1.4} is —Cl. In embodiments, R^{1.1} is —CN; R^{1.2}, R^{1.3}, and R^{1.5} are hydrogen; and R^{1.4} is halogen (e.g., —F, —Cl, —Br, or —I). In embodiments, R^{1.1} is —CN; R^{1.2}, R^{1.3}, and R^{1.5} are hydrogen; and R^{1.4} is —Cl.

In embodiments, R^{1.2} is —CN; and R^{1.3} is halogen (e.g., —F, —Cl, —Br, or —I). In embodiments, R^{1.2} is —CN; and R^{1.3} is —F. In embodiments, R^{1.1} and R^{1.5} are hydrogen; R^{1.2} is —CN; and R^{1.3} is halogen (e.g., —F, —Cl, —Br, or —I). In embodiments, R^{1.1} and R^{1.5} are hydrogen; R^{1.2} is —CN; and R^{1.3} is —F. In embodiments, R^{1.1}, R^{1.4} and R^{1.5} are hydrogen; R^{1.2} is —CN; and R^{1.3} is halogen (e.g., —F, —Cl, —Br, or —I). In embodiments, R^{1.1}, R^{1.4} and R^{1.5} are hydrogen; R^{1.2} is —CN; and R^{1.3} is —F.

In embodiments, R^{1.3} is halogen (e.g., —F, —Cl, —Br, or —I); and R^{1.4} is —CN. In embodiments, R^{1.3} is —F; and R^{1.4} is —CN. In embodiments, R^{1.1} and R^{1.5} are hydrogen; R^{1.3} is halogen (e.g., —F, —Cl, —Br, or —I); and R^{1.4} is —CN. In embodiments, R^{1.1} and R^{1.5} are hydrogen; R^{1.3} is —F; and R^{1.4} is —CN. In embodiments, R^{1.1}, R^{1.2} and R^{1.5} are hydrogen; R^{1.3} is halogen (e.g., —F, —Cl, —Br, or —I); and R^{1.4} is —CN. In embodiments, R^{1.1}, R^{1.2} and R^{1.5} are hydrogen; R^{1.3} is —F; and R^{1.4} is —CN.

In embodiments, R^{1.3} is —C(O)OCH₃; and R^{1.4} is —OCH₃. In embodiments, R^{1.1} and R^{1.5} are hydrogen; R^{1.3} is —C(O)OCH₃; and R^{1.4} is —OCH₃. In embodiments, R^{1.1}, R^{1.2}, and R^{1.5} are hydrogen; R^{1.3} is —C(O)OCH₃; and R^{1.4} is —OCH₃. In embodiments, R^{1.3} is —C(O)OCH₃; and R^{1.2} is —OCH₃. In embodiments, R^{1.1} and R^{1.5} are hydrogen; R^{1.3} is —C(O)OCH₃; and R^{1.2} is —OCH₃. In embodiments, R^{1.1}, R^{1.4}, and R^{1.5} are hydrogen; R^{1.2} is —C(O)OCH₃; and R^{1.4} is —OCH₃.

In embodiments, R^{1.3} is —C(O)OH; and R^{1.4} is —OCH₃. In embodiments, R^{1.1} and R^{1.5} are hydrogen; R^{1.3} is —C(O)OH; and R^{1.4} is —OCH₃. In embodiments, R^{1.1}, R^{1.2}, and R^{1.5} are hydrogen; R^{1.3} is —C(O)OH; and R^{1.4} is —OCH₃. In embodiments, R^{1.3} is —C(O)OH; and R^{1.2} is —OCH₃. In embodiments, R^{1.1} and R^{1.5} are hydrogen; R^{1.3} is —C(O)OH; and R^{1.2} is —OCH₃. In embodiments, R^{1.1}, R^{1.4}, and R^{1.5} are hydrogen; R^{1.2} is —C(O)OH; and R^{1.4} is —OCH₃.

In embodiments, R² is —CN,

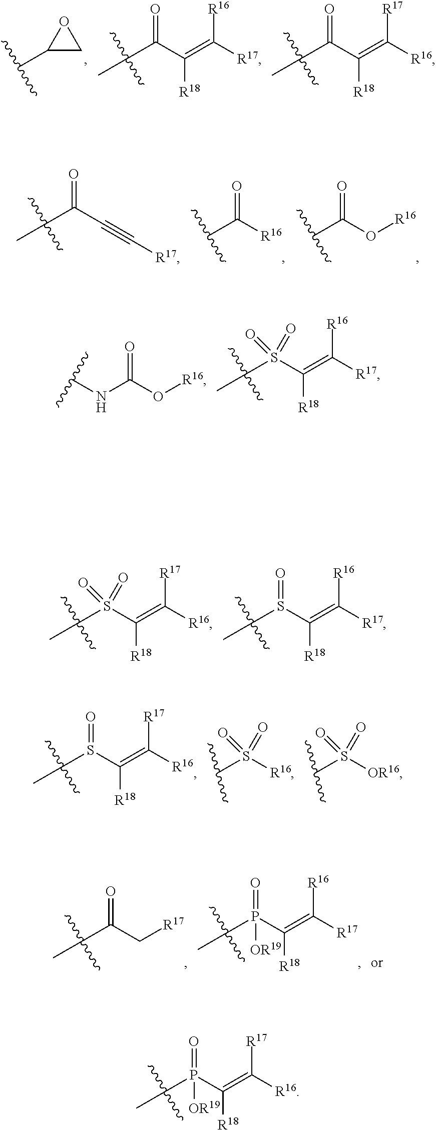

R¹⁶ is hydrogen,

halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC(O)NHNR^{16A}R^{16B},
—NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16B}, —NR^{16A}C(O)R^{16C}, —NR^{16A}C(O)OR^{16C}, —NR^{16A}OR^{16D}, —OCX¹⁶³, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁷ is hydrogen,

halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC(O)NHNR^{17A}R^{17B},
—NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17B}, —NR^{17A}C(O)R^{17C}, —NR^{17A}C(O)OR^{17C}, —NR^{7A}OR^{17D}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁸ is hydrogen,

halogen, —CX¹⁸₃, —CHX¹⁸₂, —CH₂X¹⁸, —CN, —SOₙ₁₈R^{18D}, —SOᵥ₁₈NR^{18A}R^{18B}, —NHNR^{18A}R^{18B}, —ONR^{18A}R^{18B}, —NHC(O)NHNR^{18A}R^{18B},
—NHC(O)NR^{18A}R^{18B}, —N(O)ₘ₁₈, —NR^{1A}R^{18B}, —C(O)R^{18C}, —C(O)—OR^{18C}, —C(O)NR^{18A}R^{18B}, —OR^{18D}, —NR^{18A}SO₂R^{18B}, —NR^{18A}C(O)R^{18C}, —NR^{1A}C(O)OR^{18C}, —NR^{1A}OR^{18D}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁹ is hydrogen,

halogen, —CX¹⁹₃, —CHX¹⁹₂, —CH₂X¹⁹, —CN, —SOₙ₁₉R^{19D}, —SOᵥ₁₉NR^{19A}R^{19B}, —NR^{19A}R^{19B}, —ONR^{19A}R^{19B}, —NHC(O)NHNR^{19A}R^{19B},
—NHC(O)NR^{19A}R^{19B}, —N(O)ₘ₁₉, —NR^{19A}R^{19B}, —C(O)R^{19C}, —C(O)—OR^{19C}, —C(O)NR^{19A}R^{19B}, —OR^{19D}, —NR^{19A}SO₂R^{19B}, —NR^{19A}C(O)R^{19C}, —NR^{19A}C(O)OR^{19C}, —NR^{19A}OR^{19D}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently

hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{16A} and R^{16B} substituents bonded to the same nitrogen atom may optionally be joined to form a substituted or unsubstituted heterocycloalkyl or substituted or unsubstituted heteroaryl. R^{17A} and R^{17B} substituents bonded to the same nitrogen atom may optionally be joined to form a substituted or unsubstituted heterocycloalkyl or substituted or unsubstituted heteroaryl. R^{18A} and R^{18B} substituents bonded to the same nitrogen atom may optionally be joined to form a substituted or unsubstituted heterocycloalkyl or substituted or unsubstituted heteroaryl. R^{19A} and R^{19B} substituents bonded to the same nitrogen atom may optionally be joined to form a substituted or unsubstituted heterocycloalkyl or substituted or unsubstituted heteroaryl.

X¹⁶, X¹⁷, X¹⁸, and X¹⁹ are independently —F, —Cl, —Br, or —I.

n16, n17, n18, and n19 are independently an integer from 0 to 4.

m16, m17, m18, m19, v16, v17, v18, and v19 are independently 1 or 2.

In embodiments, R^{16A} and R^{16B} substituents bonded to the same nitrogen atom are joined to form a substituted or unsubstituted 5 to 6-membered heterocycloalkyl. In embodiments, R^{16A} and R^{16B} substituents bonded to the same nitrogen atom are joined to form a substituted or unsubstituted 5-membered heterocycloalkyl. In embodiments, R^{16A} and R^{16B} substituents bonded to the same nitrogen atom are joined to form a substituted 5-membered heterocycloalkyl. In embodiments, R^{16A} and R^{16B} substituents bonded to the same nitrogen atom are joined to form an unsubstituted 5-membered heterocycloalkyl. In embodiments, R^{16A} and R^{16B} substituents bonded to the same nitrogen atom are joined to form a substituted or unsubstituted 6-membered heterocycloalkyl. In embodiments, R^{16A} and R^{16B} substituents bonded to the same nitrogen atom are joined to form a substituted 6-membered heterocycloalkyl. In embodiments, R^{16A} and R^{16B} substituents bonded to the same nitrogen atom are joined to form an unsubstituted 6-membered heterocycloalkyl.

In embodiments, R^{17A} and R^{17B} substituents bonded to the same nitrogen atom are joined to form a substituted or unsubstituted 5 to 6-membered heterocycloalkyl. In embodiments, R^{17A} and R^{17B} substituents bonded to the same nitrogen atom are joined to form a substituted or unsubstituted 5-membered heterocycloalkyl. In embodiments, R^{17A} and R^{17B} substituents bonded to the same nitrogen atom are joined to form a substituted 5-membered heterocycloalkyl. In embodiments, R^{17A} and R^{17B} substituents bonded to the same nitrogen atom are joined to form an unsubstituted 5-membered heterocycloalkyl. In embodiments, R^{17A} and R^{17B} substituents bonded to the same nitrogen atom are joined to form a substituted or unsubstituted 6-membered heterocycloalkyl. In embodiments, R^{17A} and R^{17B} substituents bonded to the same nitrogen atom are joined to form a substituted 6-membered heterocycloalkyl. In embodiments, R^{17A} and R^{17B} substituents bonded to the same nitrogen atom are joined to form an unsubstituted 6-membered heterocycloalkyl.

In embodiments, R^{18A} and R^{18B} substituents bonded to the same nitrogen atom are joined to form a substituted or unsubstituted 5 to 6-membered heterocycloalkyl. In embodiments, R^{18A} and R^{18B} substituents bonded to the same nitrogen atom are joined to form a substituted or unsubstituted 5-membered heterocycloalkyl. In embodiments, R^{18A} and R^{18B} substituents bonded to the same nitrogen atom are joined to form a substituted 5-membered heterocycloalkyl. In embodiments, R^{18A} and R^{18B} substituents bonded to the same nitrogen atom are joined to form an unsubstituted 5-membered heterocycloalkyl. In embodiments, R^{18A} and R^{18B} substituents bonded to the same nitrogen atom are joined to form a substituted or unsubstituted 6-membered heterocycloalkyl. In embodiments, R^{18A} and R^{18B} substituents bonded to the same nitrogen atom are joined to form a substituted 6-membered heterocycloalkyl. In embodiments, R^{18A} and R^{18B} substituents bonded to the same nitrogen atom are joined to form an unsubstituted 6-membered heterocycloalkyl.

In embodiments, R^{19A} and R^{19B} substituents bonded to the same nitrogen atom are joined to form a substituted or unsubstituted 5 to 6-membered heterocycloalkyl. In embodiments, R^{19A} and R^{19B} substituents bonded to the same nitrogen atom are joined to form a substituted or unsubstituted 5-membered heterocycloalkyl. In embodiments, R^{19A} and R^{19B} substituents bonded to the same nitrogen atom are joined to form a substituted 5-membered heterocycloalkyl. In embodiments, R^{19A} and R^{19B} substituents bonded to the same nitrogen atom are joined to form an unsubstituted 5-membered heterocycloalkyl. In embodiments, R^{19A} and R^{19B} substituents bonded to the same nitrogen atom are joined to form a substituted or unsubstituted 6-membered heterocycloalkyl. In embodiments, R^{19A} and R^{19B} substituents bonded to the same nitrogen atom are joined to form a substituted 6-membered heterocycloalkyl. In embodiments, R^{19A} and R^{19B} substituents bonded to the same nitrogen atom are joined to form an unsubstituted 6-membered heterocycloalkyl.

In embodiments, R² is —CN. In embodiments, R² is

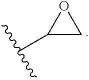 [SMILES: *C1CO1]

In embodiments, R² is

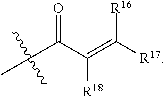

In embodiments, R² is

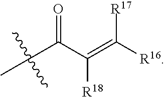

In embodiments, R² is

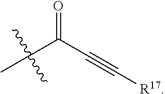

In embodiments, R² is

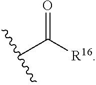

In embodiments, R² is

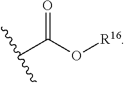

In embodiments, R² is

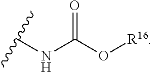

In embodiments, R² is

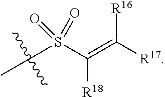

In embodiments, R² is

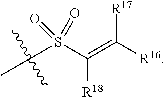

In embodiments, R² is

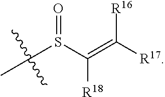

In embodiments, R² is

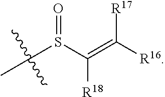

In embodiments, R² is

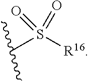

In embodiments, R² is

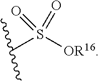

In embodiments, R² is

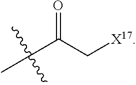

In embodiments, R² is

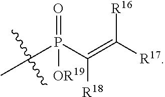

In embodiments, R² is

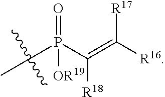

In embodiments, R¹⁶ is hydrogen, unsubstituted C₁-C₄ alkyl, or unsubstituted C₃-C₆ cycloalkyl. In embodiments, R¹⁶ is hydrogen. In embodiments, R¹⁶ is unsubstituted C₁-C₄ alkyl. In embodiments, R¹⁶ is unsubstituted C₁-C₃ alkyl. In embodiments, R¹⁶ is unsubstituted methyl. In embodiments, R¹⁶ is unsubstituted ethyl. In embodiments, R¹⁶ is unsubstituted propyl. In embodiments, R¹⁶ is unsubstituted isopropyl. In embodiments, R¹⁶ is unsubstituted butyl. In embodiments, R¹⁶ is unsubstituted isobutyl. In embodiments, R¹⁶ is unsubstituted 2-methyl propyl. In embodiments, R¹⁶ is unsubstituted t-butyl. In embodiments, R¹⁶ is unsubstituted C₃-C₆ cycloalkyl. In embodiments, R¹⁶ is unsubstituted C₃-C₅ cycloalkyl. In embodiments, R¹⁶ is unsubstituted C₃-C₄ cycloalkyl. In embodiments, R¹⁶ is unsubstituted C₅-C₆ cycloalkyl. In embodiments, R¹⁶ is unsubstituted cyclopropyl. In embodiments, R¹⁶ is unsubstituted cyclobutyl. In embodiments, R¹⁶ is unsubstituted cyclopentyl. In embodiments, R¹⁶ is unsubstituted cyclohexyl.

In embodiments, R¹⁷ is hydrogen, unsubstituted C₁-C₄ alkyl, or unsubstituted C₃-C₆ cycloalkyl. In embodiments, R¹⁷ is hydrogen. In embodiments, R¹⁷ is unsubstituted C₁-C₄ alkyl. In embodiments, R¹⁷ is unsubstituted C₁-C₃ alkyl. In embodiments, R¹⁷ is unsubstituted methyl. In embodiments, R¹⁷ is unsubstituted ethyl. In embodiments, R¹⁷ is unsubstituted propyl. In embodiments, R¹⁷ is unsubstituted isopropyl. In embodiments, R¹⁷ is unsubstituted butyl. In embodiments, R¹⁷ is unsubstituted isobutyl. In embodiments, R¹⁷ is unsubstituted 2-methyl propyl. In embodiments, R¹⁷ is unsubstituted t-butyl. In embodiments, R¹⁷ is unsubstituted C₃-C₆ cycloalkyl. In embodiments, R¹⁷ is unsubstituted C₃-C₅ cycloalkyl. In embodiments, R¹⁷ is unsubstituted C₃-C₄ cycloalkyl. In embodiments, R¹⁷ is unsubstituted C₅-C₆ cycloalkyl. In embodiments, R¹⁷ is unsubstituted cyclopropyl. In embodiments, R¹⁷ is unsubstituted cyclobutyl. In embodiments, R¹⁷ is unsubstituted cyclopentyl. In embodiments, R¹⁷ is unsubstituted cyclohexyl.

In embodiments, R¹⁸ is hydrogen, unsubstituted C₁-C₄ alkyl, or unsubstituted C₃-C₆ cycloalkyl. In embodiments, R¹⁸ is hydrogen. In embodiments, R¹⁸ is unsubstituted C₁-C₄ alkyl. In embodiments, R¹⁸ is unsubstituted C₁-C₃ alkyl. In embodiments, R¹⁸ is unsubstituted methyl. In embodiments, R¹⁸ is unsubstituted ethyl. In embodiments, R¹⁸ is unsubstituted propyl. In embodiments, R¹⁸ is unsubstituted isopropyl. In embodiments, R¹⁸ is unsubstituted butyl. In embodiments, R¹⁸ is unsubstituted isobutyl. In embodiments, R¹⁸ is unsubstituted 2-methyl propyl. In embodiments, R¹⁸ is unsubstituted t-butyl. In embodiments, R¹⁸ is unsubstituted C₃-C₆ cycloalkyl. In embodiments, R¹⁸ is unsubstituted C₃-C₅ cycloalkyl. In embodiments, R¹⁸ is unsubstituted C₃-C₄ cycloalkyl. In embodiments, R¹⁸ is unsubstituted C₅-C₆ cycloalkyl. In embodiments, R¹⁸ is unsubstituted cyclopropyl. In embodiments, R¹⁸ is unsubstituted cyclobutyl. In embodiments, R¹⁸ is unsubstituted cyclopentyl. In embodiments, R¹⁸ is unsubstituted cyclohexyl.

In embodiments, R¹⁶ is hydrogen or unsubstituted C₁-C₄ alkyl; R¹⁷ is hydrogen or unsubstituted C₁-C₄ alkyl; and R¹⁸ is hydrogen or unsubstituted C₁-C₄ alkyl.

In embodiments, R¹⁶ is hydrogen; R¹⁷ is hydrogen or unsubstituted C₁-C₄ alkyl; and R¹⁸ is hydrogen or unsubstituted C₁-C₄ alkyl. In embodiments, R¹⁶ is unsubstituted C₁-C₄ alkyl; R¹⁷ is hydrogen or unsubstituted C₁-C₄ alkyl; and R¹⁸ is hydrogen or unsubstituted C₁-C₄ alkyl. In embodiments, R¹⁶ is hydrogen or unsubstituted C₁-C₄ alkyl; R¹⁷ is hydrogen; and R¹⁸ is hydrogen or unsubstituted C₁-C₄ alkyl. In embodiments, R¹⁶ is hydrogen or unsubstituted C₁-C₄ alkyl; R¹⁷ is unsubstituted C₁-C₄ alkyl; and R¹⁸ is hydrogen or unsubstituted C₁-C₄ alkyl. In embodiments, R¹⁶ is hydrogen or unsubstituted C₁-C₄ alkyl; R¹⁷ is hydrogen or unsubstituted C₁-C₄ alkyl; and R¹⁸ is hydrogen. In embodiments, R¹⁶ is hydrogen or unsubstituted C₁-C₄ alkyl; R¹⁷ is hydrogen or unsubstituted C₁-C₄ alkyl; and R¹⁸ is unsubstituted C₁-C₄ alkyl.

In embodiments, R¹⁶ is hydrogen; R¹⁷ is hydrogen; and R¹⁸ is hydrogen or unsubstituted C₁-C₄ alkyl. In embodiments, R¹⁶ is hydrogen; R¹⁷ is unsubstituted C₁-C₄ alkyl; and R¹⁸ is hydrogen or unsubstituted C₁-C₄ alkyl. In embodiments, R¹⁶ is unsubstituted C₁-C₄ alkyl; R¹⁷ is hydrogen; and R¹⁸ is hydrogen or unsubstituted C₁-C₄ alkyl. In embodiments, R¹⁶ is unsubstituted C₁-C₄ alkyl; R¹⁷ is unsubstituted C₁-C₄ alkyl; and R¹⁸ is hydrogen or unsubstituted C₁-C₄ alkyl. In embodiments, R¹⁶ is hydrogen; R¹⁷ is hydrogen; and R¹⁸ is hydrogen. In embodiments, R¹⁶ is hydrogen; R¹⁷ is hydrogen; and R¹⁸ is unsubstituted C₁-C₄ alkyl. In embodiments, R¹⁶ is hydrogen; R¹⁷ is unsubstituted C₁-C₄ alkyl; and R¹⁸ is hydrogen. In embodiments, R¹⁶ is hydrogen; R¹⁷ is unsubstituted C₁-C₄ alkyl; and R¹⁸ is unsubstituted C₁-C₄ alkyl. In embodiments, R¹⁶ is unsubstituted C₁-C₄ alkyl; R¹⁷ is hydrogen; and R¹⁸ is hydrogen. In embodiments, R¹⁶ is unsubstituted C₁-C₄ alkyl; R¹⁷ is hydrogen; and R¹⁸ is unsubstituted C₁-C₄ alkyl. In embodiments, R¹⁶ is unsubstituted C₁-C₄ alkyl; R¹⁷ is unsubstituted C₁-C₄ alkyl; and R¹⁸ is hydrogen. In embodiments, R¹⁶ is unsubstituted C₁-C₄ alkyl; R¹⁷ is unsubstituted C₁-C₄ alkyl; and R¹⁸ is unsubstituted C₁-C₄ alkyl. In embodiments, R¹⁶, R¹⁷, and R¹⁸ are hydrogen.

In embodiments is provided a compound having the formula

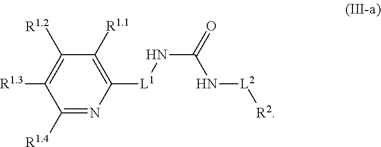

L¹, L², R^{1.1}, R^{1.2}, R^{1.3}, R^{1.4} and R² are described herein.

In embodiments is provided a compound having the formula

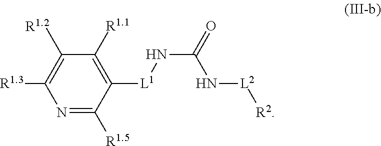

L¹, L², R^{1.1}, R^{1.2}, R^{1.3}, R^{1.5} and R² are described herein.

In embodiments, R^{1.3} is substituted or unsubstituted 2 to 6 membered heteroalkyl. In embodiments, R^{1.3} is —CN. In embodiments, R^{1.1} and R^{1.5} are hydrogen; and R^{1.3} is —CN. In embodiments, R^{1.1} and R^{1.5} are hydrogen; and R^{1.3} is —CN. In embodiments, R^{1.1}, R^{1.4} and R^{1.5} are hydrogen; and R^{1.3} is —CN. In embodiments, R^{1.1}, R^{1.2} and R^{1.5} is hydrogen; and R^{1.3} is —CN.

In embodiments is provided a compound having the formula

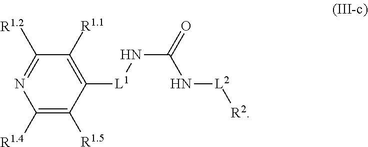

L¹, L², R^{1.1}, R^{1.2}, R^{1.4}, R^{1.5} and R² are described herein.

In embodiments is provided a compound having the formula

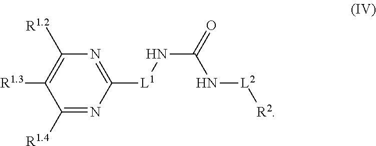

L¹, L², R^{1.2}, R^{1.3}, R^{1.4} and R² are described herein.

In embodiments, R^{1.2} is substituted or unsubstituted alkyl. In embodiments, R^{1.2} is hydrogen. In embodiments, R^{1.2} is substituted or unsubstituted alkyl. In embodiments, R^{1.2} is substituted alkyl. In embodiments, R^{1.2} is unsubstituted alkyl. In embodiments, R^{1.2} is substituted or unsubstituted C₁-C₆ alkyl. In embodiments, R^{1.2} is unsubstituted C₁-C₆ alkyl. In embodiments, R^{1.2} is substituted or unsubstituted C₁-C₅ alkyl. In embodiments, R^{1.2} is unsubstituted C₁-C₅ alkyl. In embodiments, R^{1.2} is substituted or unsubstituted C₁-C₄ alkyl. In embodiments, R^{1.2} is unsubstituted C₁-C₄ alkyl. In embodiments, R^{1.2} is methyl. In embodiments, R^{1.2} is ethyl. In embodiments, R^{1.2} is propyl. In embodiments, R^{1.2} is isopropyl. In embodiments, R^{1.2} is butyl. In embodiments, R^{1.2} is t-butyl.

In embodiments, R^{1.2} is —OR^{1D}. In embodiments, R^{1D} is hydrogen, or substituted or unsubstituted C₁-C₄ alkyl. In embodiments, R^{1D} is hydrogen. In embodiments, R^{1D} is substituted or unsubstituted C₁-C₄ alkyl. In embodiments, R^{1D} is unsubstituted C₁-C₄ alkyl. In embodiments, R^{1D} is methyl. In embodiments, R^{1D} is ethyl. In embodiments, R^{1D} is propyl. In embodiments, R^{1D} is isopropyl. In embodiments, R^{1D} is butyl. In embodiments, R^{1D} is t-butyl. In embodiments, R^{1.2} is —OH. In embodiments, R^{1.2} is —OCH₃. In embodiments, R^{1.2} is —OCH₂CH₃.

In embodiments, R^{1.4} is substituted or unsubstituted alkyl. In embodiments, R^{1.4} is hydrogen. In embodiments, R^{1.4} is substituted or unsubstituted alkyl. In embodiments, R^{1.4} is substituted alkyl. In embodiments, R^{1.4} is unsubstituted alkyl. In embodiments, R^{1.4} is substituted or unsubstituted C₁-C₆ alkyl. In embodiments, R^{1.4} is unsubstituted C₁-C₆ alkyl. In embodiments, R^{1.4} is substituted or unsubstituted C₁-C₅ alkyl. In embodiments, R^{1.4} is unsubstituted C₁-C₅ alkyl. In embodiments, R^{1.4} is substituted or unsubstituted C₁-C₄ alkyl. In embodiments, R^{1.4} is unsubstituted C₁-C₄ alkyl. In embodiments, R^{1.4} is methyl. In embodiments, R^{1.4} is ethyl. In embodiments, R^{1.4} is propyl. In embodiments, R^{1.4} is isopropyl. In embodiments, R^{1.4} is butyl. In embodiments, R^{1.4} is t-butyl.

In embodiments, R^{1.4} is —OR^{1D}. In embodiments, R^{1D} is hydrogen, or substituted or unsubstituted C₁-C₄ alkyl. In embodiments, R^{1D} is hydrogen. In embodiments, R^{1D} is substituted or unsubstituted C₁-C₄ alkyl. In embodiments, R^{1D} is unsubstituted C₁-C₄ alkyl. In embodiments, R^{1D} is methyl. In embodiments, R^{1D} is ethyl. In embodiments, R^{1D} is propyl. In embodiments, R^{1D} is isopropyl. In embodiments, R^{1D} is butyl. In embodiments, R^{1D} is t-butyl. In embodiments, R^{1.4} is —OH. In embodiments, R^{1.4} is —OCH₃. In embodiments, R^{1.4} is —OCH₂CH₃.

In embodiments, R^{1.2} is unsubstituted C₁-C₄ alkyl and R^{1.4} is —OR^{1D}. In embodiments, R^{1.2} is methyl and R^{1.4} is —OH. In embodiments, R^{1.2} is methyl and R^{1.4} is —OCH₃. In embodiments, R^{1.2} is ethyl and R^{1.4} is —OCH₃. In embodiments, R^{1.2} is ethyl and R^{1.4} is —OCH₂CH₃. In embodiments, R^{1.2} is —OR^{1D} and R^{1.4} is unsubstituted C₁-C₄ alkyl. In embodiments, R^{1.2} is —OH and R^{1.4} is methyl. In embodiments, R^{1.2} is —OCH₃ and R^{1.4} is methyl. In embodiments, R^{1.2} is —OCH₃ and R^{1.4} is ethyl. In embodiments, R^{1.2} is —OCH₂CH₃ and R^{1.4} is ethyl.

In embodiments is provided a compound having the formula

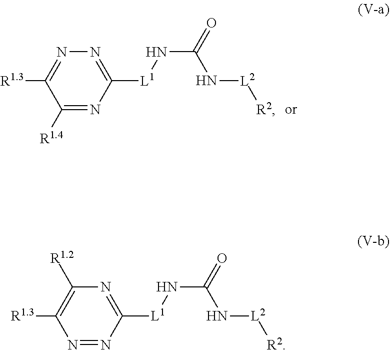

L¹, L², R^{1.2}, R^{1.3}, R⁴ and R² are described herein.

In embodiments, R^{1.2} is substituted or unsubstituted alkyl. In embodiments, R^{1.2} is hydrogen. In embodiments, R^{1.2} is substituted or unsubstituted alkyl. In embodiments, R^{1.2} is substituted alkyl. In embodiments, R^{1.2} is unsubstituted alkyl. In embodiments, R^{1.2} is substituted or unsubstituted C₁-C₆ alkyl. In embodiments, R^{1.2} is unsubstituted C₁-C₆ alkyl. In embodiments, R^{1.2} is substituted or unsubstituted C₁-C₅ alkyl. In embodiments, R^{1.2} is unsubstituted C₁-C₅ alkyl. In embodiments, R^{1.2} is substituted or unsubstituted C₁-C₄ alkyl. In embodiments, R^{1.2} is unsubstituted C₁-C₄ alkyl. In embodiments, R^{1.2} is methyl. In embodiments, R^{1.2} is ethyl. In embodiments, R^{1.2} is propyl. In embodiments, R^{1.2} is isopropyl. In embodiments, R^{1.2} is butyl. In embodiments, R^{1.2} is t-butyl.

In embodiments, R^{1.3} is substituted or unsubstituted alkyl. In embodiments, R^{1.3} is hydrogen. In embodiments, R^{1.3} is substituted or unsubstituted alkyl. In embodiments, R^{1.3} is substituted alkyl. In embodiments, R^{1.3} is unsubstituted alkyl. In embodiments, R^{1.3} is substituted or unsubstituted C₁-C₆ alkyl. In embodiments, R^{1.3} is unsubstituted C₁-C₆ alkyl. In embodiments, R^{1.3} is substituted or unsubstituted C₁-C₅ alkyl. In embodiments, R^{1.3} is unsubstituted C₁-C₅ alkyl. In embodiments, R^{1.3} is substituted or unsubstituted C₁-C₄ alkyl. In embodiments, R^{1.3} is unsubstituted C₁-C₄ alkyl. In embodiments, R^{1.3} is methyl. In embodiments, R^{1.3} is ethyl. In embodiments, R^{1.3} is propyl. In embodiments, R^{1.3} is isopropyl. In embodiments, R^{1.3} is butyl. In embodiments, R^{1.3} is t-butyl.

In embodiments, R^{1.4} is substituted or unsubstituted alkyl. In embodiments, R^{1.4} is hydrogen. In embodiments, R^{1.4} is substituted or unsubstituted alkyl. In embodiments, R^{1.4} is substituted alkyl. In embodiments, R^{1.4} is unsubstituted alkyl. In embodiments, R^{1.4} is substituted or unsubstituted C₁-C₆ alkyl. In embodiments, R^{1.4} is unsubstituted C₁-C₆ alkyl. In embodiments, R^{1.4} is substituted or unsubstituted C₁-C₅ alkyl. In embodiments, R^{1.4} is unsubstituted C₁-C₅ alkyl. In embodiments, R^{1.4} is substituted or unsubstituted C₁-C₄ alkyl. In embodiments, R^{1.4} is unsubstituted C₁-C₄ alkyl. In embodiments, R^{1.4} is methyl. In embodiments, R^{1.4} is ethyl. In embodiments, R^{1.4} is propyl. In embodiments, R^{1.4} is isopropyl. In embodiments, R^{1.4} is butyl. In embodiments, R^{1.4} is t-butyl.

In embodiments, R^{1.2} and R^{1.3} are independently hydrogen or unsubstituted C₁-C₄ alkyl. In embodiments, R^{1.2} and R^{1.3} are independently unsubstituted C₁-C₄ alkyl. In embodiments, R^{1.2} and R^{1.3} are independently methyl or ethyl. In embodiments, R^{1.2} and R^{1.3} are methyl. In embodiments, R^{1.3} and R^{1.4} are independently hydrogen or unsubstituted C₁-C₄ alkyl. In embodiments, R^{1.3} and R^{1.4} are independently unsubstituted C₁-C₄ alkyl. In embodiments, R^{1.3} and R^{1.4} are independently methyl or ethyl. In embodiments, R^{1.3} and R^{1.4} are methyl.

In embodiments is provided a compound having the formula

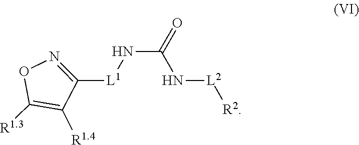

L¹, L², R^{1.3}, R^{1.4} and R² are described herein.

In embodiments, R^{1.3} is hydrogen, or substituted or unsubstituted alkyl. In embodiments, R^{1.3} is substituted or unsubstituted alkyl. In embodiments, R^{1.3} is hydrogen. In embodiments, R^{1.3} is substituted or unsubstituted alkyl. In embodiments, R^{1.3} is substituted alkyl. In embodiments, R^{1.3} is unsubstituted alkyl. In embodiments, R^{1.3} is substituted or unsubstituted C₁-C₆ alkyl. In embodiments, R^{1.3} is unsubstituted C₁-C₆ alkyl. In embodiments, R^{1.3} is substituted or unsubstituted C₁-C₅ alkyl. In embodiments, R^{1.3} is unsubstituted C₁-C₅ alkyl. In embodiments, R^{1.3} is substituted or unsubstituted C₁-C₄ alkyl. In embodiments, R^{1.3} is unsubstituted C₁-C₄ alkyl. In embodiments, R^{1.3} is methyl. In embodiments, R^{1.3} is ethyl. In embodiments, R^{1.3} is propyl. In embodiments, R^{1.3} is isopropyl. In embodiments, R^{1.3} is butyl. In embodiments, R^{1.3} is t-butyl. In embodiments, R^{1.3} is hydrogen.

In embodiments, R^{1.4} is hydrogen, or substituted or unsubstituted alkyl. In embodiments, R^{1.4} is substituted or unsubstituted alkyl. In embodiments, R^{1.4} is hydrogen. In embodiments, R^{1.4} is substituted or unsubstituted alkyl. In embodiments, R^{1.4} is substituted alkyl. In embodiments, R^{1.4} is unsubstituted alkyl. In embodiments, R^{1.4} is substituted or unsubstituted C₁-C₆ alkyl. In embodiments, R^{1.4} is unsubstituted C₁-C₆ alkyl. In embodiments, R^{1.4} is substituted or unsubstituted C₁-C₅ alkyl. In embodiments, R^{1.4} is unsubstituted C₁-C₅ alkyl. In embodiments, R^{1.4} is substituted or unsubstituted C₁-C₄ alkyl. In embodiments, R^{1.4} is unsubstituted C₁-C₄ alkyl. In embodiments, R^{1.4} is methyl. In embodiments, R^{1.4} is ethyl. In embodiments, R^{1.4} is propyl. In embodiments, R^{1.4} is isopropyl. In embodiments, R^{1.4} is butyl. In embodiments, R^{1.4} is t-butyl. In embodiments, R^{1.4} is hydrogen.

In embodiments, R^{1.3} and R^{1.4} are independently hydrogen or unsubstituted C₁-C₄ alkyl. In embodiments, R^{1.3} is hydrogen and R^{1.4} is unsubstituted C₁-C₄ alkyl. In embodiments, R^{1.3} is unsubstituted C₁-C₄ alkyl and R^{1.4} is hydrogen. In embodiments, R^{1.3} is hydrogen and R^{1.4} is methyl or ethyl. In embodiments, R^{1.3} is methyl or ethyl and R^{1.4} is hydrogen.

In embodiments is provided a compound having the formula

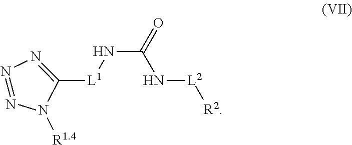

L¹, L², R^{1.4} and R² are described herein.

In embodiments, R^{1.4} is hydrogen, or substituted or unsubstituted alkyl. In embodiments, R^{1.4} is substituted or unsubstituted alkyl. In embodiments, R^{1.4} is hydrogen. In embodiments, R^{1.4} is substituted or unsubstituted alkyl. In embodiments, R^{1.4} is substituted alkyl. In embodiments, R^{1.4} is unsubstituted alkyl. In embodiments, R^{1.4} is substituted or unsubstituted C₁-C₆ alkyl. In embodiments, R^{1.4} is unsubstituted C₁-C₆ alkyl. In embodiments, R^{1.4} is substituted or unsubstituted C₁-C₅ alkyl. In embodiments, R^{1.4} is unsubstituted C₁-C₅ alkyl. In embodiments, R^{1.4} is substituted or unsubstituted C₁-C₄ alkyl. In embodiments, R^{1.4} is unsubstituted C₁-C₄ alkyl. In embodiments, R^{1.4} is methyl. In embodiments, R^{1.4} is ethyl. In embodiments, R^{1.4} is propyl. In embodiments, R^{1.4} is isopropyl. In embodiments, R^{1.4} is butyl. In embodiments, R^{1.4} is t-butyl. In embodiments, R^{1.4} is hydrogen.

In embodiments is provided a compound having the formula

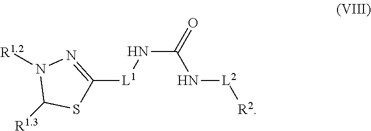

L¹, L², R^{1.2}, R^{1.3} and R² are described herein.

In embodiments, R^{1.2} is hydrogen, or substituted or unsubstituted alkyl. In embodiments, R^{1.2} is substituted or unsubstituted alkyl. In embodiments, R^{1.2} is hydrogen. In embodiments, R^{1.2} is substituted or unsubstituted alkyl. In embodiments, R^{1.2} is substituted alkyl. In embodiments, R^{1.2} is unsubstituted alkyl. In embodiments, R^{1.2} is substituted or unsubstituted C₁-C₆ alkyl. In embodiments, R^{1.2} is unsubstituted C₁-C₆ alkyl. In embodiments, R^{1.2} is substituted or unsubstituted C₁-C₅ alkyl. In embodiments, R^{1.2} is unsubstituted C₁-C₅ alkyl. In embodiments, R^{1.2} is substituted or unsubstituted C₁-C₄ alkyl. In embodiments, R^{1.2} is unsubstituted C₁-C₄ alkyl. In embodiments, R^{1.2} is methyl. In embodiments, R^{1.2} is ethyl. In embodiments, R^{1.2} is propyl. In embodiments, R^{1.2} is isopropyl. In embodiments, R^{1.2} is butyl. In embodiments, R^{1.2} is t-butyl. In embodiments, R^{1.2} is hydrogen.

In embodiments, R^{1.3} is hydrogen, or substituted or unsubstituted alkyl. In embodiments, R^{1.3} is substituted or unsubstituted alkyl. In embodiments, R^{1.3} is hydrogen. In embodiments, R^{1.3} is substituted or unsubstituted alkyl. In embodiments, R^{1.3} is substituted alkyl. In embodiments, R^{1.3} is unsubstituted alkyl. In embodiments, R^{1.3} is substituted or unsubstituted C₁-C₆ alkyl. In embodiments, R^{1.3} is unsubstituted C₁-C₆ alkyl. In embodiments, R^{1.3} is substituted or unsubstituted C₁-C₅ alkyl. In embodiments, R^{1.3} is unsubstituted C₁-C₅ alkyl. In embodiments, R^{1.3} is substituted or unsubstituted C₁-C₄ alkyl. In embodiments, R^{1.3} is unsubstituted C₁-C₄ alkyl. In embodiments, R^{1.3} is methyl. In embodiments, R^{1.3} is ethyl. In embodiments, R^{1.3} is propyl. In embodiments, R^{1.3} is isopropyl. In embodiments, R^{1.3} is butyl. In embodiments, R^{1.3} is t-butyl. In embodiments, R^{1.3} is hydrogen.

In embodiments, R^{1.2} and R^{1.3} are independently hydrogen or unsubstituted C₁-C₄ alkyl. In embodiments, R^{1.2} is hydrogen and R^{1.3} is unsubstituted C₁-C₄ alkyl. In embodiments, R^{1.2} is hydrogen and R^{1.3} is methyl. In embodiments, R^{1.2} is hydrogen and R^{1.3} is ethyl. In embodiments, R^{1.2} is hydrogen and R^{1.3} is propyl. In embodiments, R^{1.2} is hydrogen and R^{1.3} is isopropyl. In embodiments, R^{1.2} is hydrogen and R^{1.3} is butyl. In embodiments, R^{1.2} is hydrogen and R^{1.3} is t-butyl. In embodiments, R^{1.2} is unsubstituted C₁-C₄alkyl and R^{1.3} is hydrogen. In embodiments, R^{1.2} is methyl and R^{1.3} is hydrogen. In embodiments, R^{1.2} is ethyl and R^{1.3} is hydrogen. In embodiments, R^{1.2} is propyl and R^{1.3} is hydrogen. In embodiments, R^{1.2} is isopropyl and R^{1.3} is hydrogen. In embodiments, R^{1.2} is butyl and R^{1.3} is hydrogen. In embodiments, R^{1.2} is t-butyl and R^{1.3} is hydrogen.

In embodiments, examples of

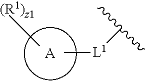

moiety including following structure:

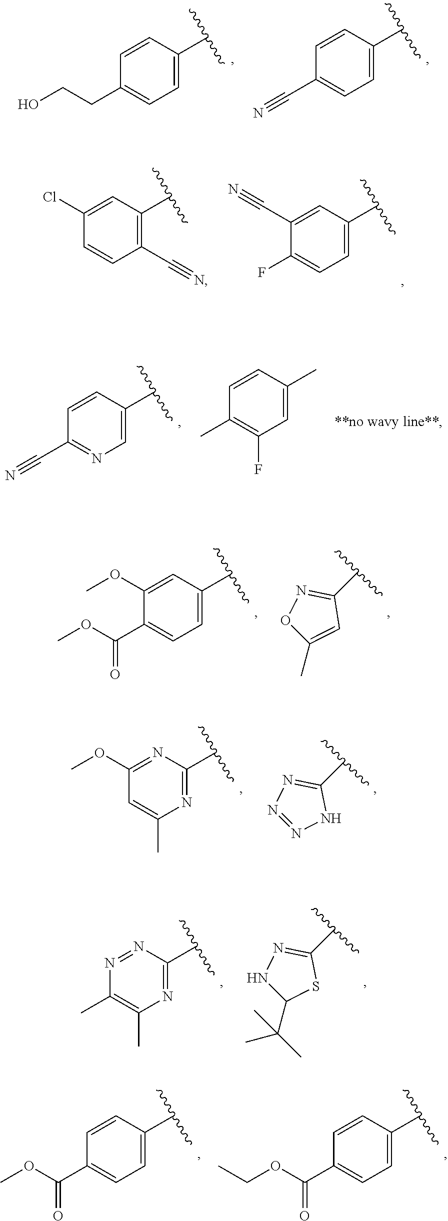

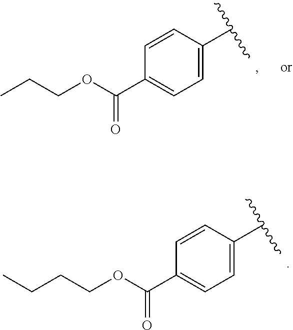 [SMILES: *c1ccc(C(=O)OCCC)cc1 ; *c1ccc(C(=O)OCCCC)cc1]

In embodiments,

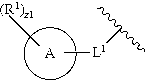

moiety in Formula (I) is

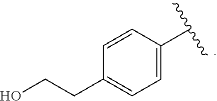 [SMILES: *c1ccc(CCO)cc1]

In embodiments,

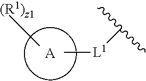

moiety in Formula (I) is

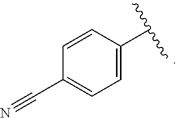 [SMILES: *c1ccc(C#N)cc1]

In embodiments,

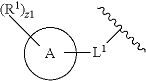

moiety in Formula (I) is

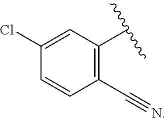 [SMILES: *c1cc(Cl)ccc1C#N]

In embodiments,

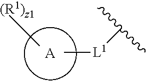

moiety in Formula (I) is

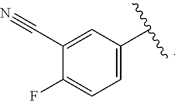 [SMILES: *c1ccc(F)c(C#N)c1]

In embodiments,

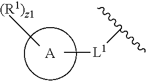

moiety in Formula (I) is

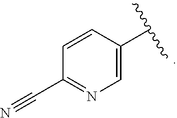 [SMILES: *c1ccc(C#N)nc1]

In embodiments,

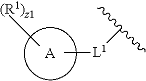

moiety in Formula (I) is

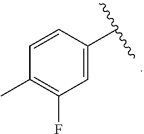 [SMILES: *c1ccc(C)c(F)c1]

In embodiments,

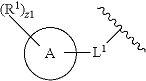

moiety in Formula (I) is

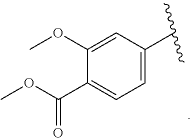 [SMILES: *c1ccc(C(=O)OC)c(OC)c1]

In embodiments,

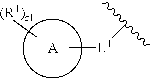

moiety in Formula (I) is

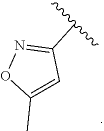 [SMILES: *c1cc(C)on1]

In embodiments,

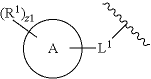

moiety in Formula (I) is

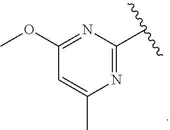 [SMILES: *c1nc(C)cc(OC)n1]

In embodiments,

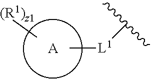

moiety in Formula (I) is

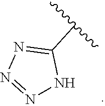 [SMILES: *c1nnn[nH]1]

In embodiments,

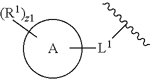

moiety in Formula (I) is

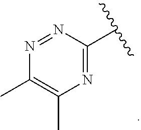 [SMILES: *c1nnc(C)c(C)n1]

In embodiments,

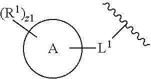

moiety in Formula (I) is

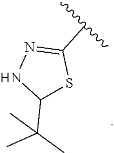 [SMILES: *C1=NNC(C(C)(C)C)S1]

In embodiments,

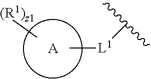

moiety in Formula (I) is

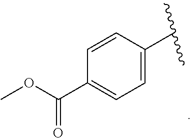 [SMILES: *c1ccc(C(=O)OC)cc1]

In embodiments,

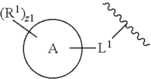

moiety in Formula (I) is

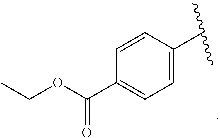 [SMILES: *c1ccc(C(=O)OCC)cc1]

In embodiments,

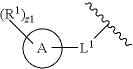

moiety in Formula (I) is

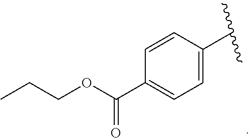 [SMILES: *c1ccc(C(=O)OCCC)cc1]

In embodiments,

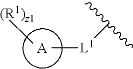

moiety in Formula (I) is

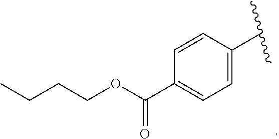 [SMILES: *c1ccc(C(=O)OCCCC)cc1]

In embodiments, R¹ is independently halogen (e.g., —F, —Cl, —Br, or —I), —CX¹₃ (e.g., —CF₃, —CCl₃, —CBr₃, or —C₃), —CHX¹₂ (e.g., —CHF₂, —CHCl₂, —CHBr₂, or —CHI₂), —CH₂X¹ (e.g., —CH₂F, —CH₂Cl, —CH₂Br, or —CH₂I), —OCX¹₃ (e.g., —OCF₃, —OCCl₃, —OCBr₃, or —OC₃), —OCH₂X¹ (e.g., —OCH₂F, —OCH₂Cl, —OCH₂Br, or —OCH₂I), —OCHX¹₂ (e.g., —OCHF₂, —OCHCl₂, —OCHBr₂, —OCHI₂), —CN, —SOₙ₁R^{1D} (e.g., —SH, —SCH₃, —SO₂H, —SO₃H, or —SO₄H), —SOᵥ₁NR^{1A}R^{1B} (e.g., —SO₂NH₂, or —SO₂NHCH₃), —NR^{1C}NR^{1A}R^{1B} (e.g., NHNH₂ or NHNHCH₃), —ONR^{1A}R^{1B} (e.g., —ONH₂, or —ONHCH₃), —NHC(O)NR^{1C}NR^{1A}R^{1B} (e.g., —NHC(O)NHNH₂, or —NHC(O)NHNHCH₃), —NHC(O)NR^{1A}R^{1B} (e.g., —NHC(O)NH₂, or —NHC(O)NHCH₃), —N(O)ₘ₁ (e.g., —NO, or —NO₂), —NR^{1A}R^{1B} (e.g., —NH₂, or —NHCH₃), —C(O)R^{1C} (e.g., —C(O)H or —C(O)CH₃), —C(O)—OR^{1C} (e.g., —C(O)OH or —C(O)OCH₃), —C(O)NR^{1A}R^{1B} (e.g., —C(O)NH₂ or —C(O) NHCH₃), —OR^{1D} (e.g., —OH, or —OCH₃), —NR^{1A}SO₂R^{1D} (e.g., —NHSO₂H), —NR^{1A}C(O)R^{1C} (e.g., —NHCOH), —NR^{1A}C(O)OR^{1C} (e.g., —NHC(O)OH), —NR^{11A}OR^{1C} (e.g., —NHOH), —N₃, substituted (e.g., substituted with a substituent group, a size-limited substituent group, or lower substituent group) or unsubstituted alkyl (e.g., C₁-C₈, C₁-C₆, or C₁-C₄ alkyl), substituted (e.g., substituted with a substituent group, a size-limited substituent group, or lower substituent group) or unsubstituted heteroalkyl (e.g., 2 to 8 membered, 2 to 6 membered, or 2 to 4 membered heteroalkyl), substituted (e.g., substituted with a substituent group, a size-limited substituent group, or lower substituent group) or unsubstituted cycloalkyl (e.g., C₃-C₈, C₃-C₆, or C₅-C₆ cycloalkyl), substituted (e.g., substituted with a substituent group, a size-limited substituent group, or lower substituent group) or unsubstituted heterocycloalkyl (e.g., 3 to 8 membered, 3 to 6 membered, or 5 to 6 membered heterocycloalkyl), substituted (e.g., substituted with a substituent group, a size-limited substituent group, or lower substituent group) or unsubstituted aryl (e.g., C₆-C₁₀, C₁₀ aryl, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered heteroaryl).

In embodiments, a substituted R¹ is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, each R¹ is independently —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{1E}-substituted or unsubstituted alkyl (e.g., C₁-C₂₀, C₁-C₁₂, C₁-C₈, C₁-C₆, C₁-C₄, or C₁-C₂), R^{1E}-substituted or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), R^{1E}-substituted or unsubstituted cycloalkyl (e.g., C₃-C₁₀, C₃-C₈, C₃-C₆, C₄-C₆, or C₅-C₆), R^{1E}-substituted or unsubstituted heterocycloalkyl (e.g., 3 to 10 membered, 3 to 8 membered, 3 to 6 membered, 4 to 6 membered, 4 to 5 membered, or 5 to 6 membered), R^{1E}-substituted or unsubstituted aryl (e.g., C₆-C₁₂, C₆-C₁₀, or phenyl), or R^{1E}-substituted or unsubstituted heteroaryl (e.g., 5 to 12 membered, 5 to 10 membered, 5 to 9 membered, or 5 to 6 membered). In embodiments, each R¹ is independently —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{1E}-substituted alkyl (e.g., C₁-C₂₀, C₁-C₁₂, C₁-C₈, C₁-C₆, C₁-C₄, or C₁-C₂), R^{1E}-substituted heteroalkyl (e.g., 2 to 20 membered, 2 to 12 membered, 2 to 8 membered, 2 to 6 membered, 4 to 6 membered, 2 to 3 membered, or 4 to 5 membered), R^{1E}-substituted cycloalkyl (e.g., C₃-C₁₀, C₃-C₈, C₃-C₆, C₄-C₆, or C₅-C₆), R^{1E}-substituted heterocycloalkyl (e.g., 3 to 10 membered, 3 to 8 membered, 3 to 6 membered, 4 to 6 membered, 4 to 5 membered, or 5 to 6 membered), R^{1E}-substituted aryl (e.g., C₆-C₁₂, C₆-C₁₀, or phenyl), or R^{1E}-substituted heteroaryl (e.g., 5 to 12 membered, 5 to 10 membered, 5 to 9 membered, or 5 to 6 membered). In embodiments, each R¹ is independently —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

Each R^{1.1}, R^{1.2}, R^{1.3}, R^{1.4}, and R^{1.5} is independently hydrogen, halogen (e.g., —F, —Cl, —Br, or —I), —CX¹₃ (e.g., —CF₃, —CCl₃, —CBr₃, or —C₃), —CHX¹₂ (e.g., —CHF₂, —CHCl₂, —CHBr₂, or —CHI₂), —CH₂X¹ (e.g., —CH₂F, —CH₂Cl, —CH₂Br, or —CH₂I), —OCX¹₃ (e.g., —OCF₃, —OCCl₃, —OCBr₃, or —OCI₃), —OCH₂X¹ (e.g., —OCH₂F, —OCH₂Cl, —OCH₂Br, or —OCH₂I), —OCHX¹₂ (e.g., —OCHF₂, —OCHCl₂, —OCHBr₂, —OCHI₂), —CN, —SOₙ₁R^{1D} (e.g., —SH, —SCH₃, —SO₂H, —SO₃H, or —SO₄H), —SOᵥ₁NR^{1A}R^{1B} (e.g., —SO₂NH₂, or —SO₂NHCH₃), —NR^{1C}NR^{1A}R^{1B} (e.g., NHNH₂ or NHNHCH₃), —ONR^{1A}R^{1B} (e.g., —ONH₂, or —ONHCH₃), —NHC(O)NR^{1C}NR^{1A}R^{1B} (e.g., —NHC(O)NHNH₂, or —NHC(O)NHNHCH₃), —NHC(O)NR^{1A}R^{1B} (e.g., —NHC(O)NH₂, or —NHC(O)NHCH₃), —N(O)ₘ₁ (e.g., —NO, or —NO₂), —NR^{1A}R^{1B} (e.g., —NH₂, or —NHCH₃), —C(O)R^{1C} (e.g., —C(O)H or —C(O)CH₃), —C(O)—OR^{1C} (e.g., —C(O)OH or —C(O)OCH₃), —C(O)NR^{1A}R^{1B} (e.g., —C(O)NH₂ or —C(O) NHCH₃), —OR^{1D} (e.g., —OH, or —OCH₃), —NR^{1A}SO₂R^{1D} (e.g., —NHSO₂H), —NR^{1A}C(O)R^{1C} (e.g., —NHCOH), —NR^{1A}C(O)OR^{1C} (e.g., —NHC(O)OH), —NR^{11A}OR^{1C} (e.g., —NHOH), —N₃, substituted (e.g., substituted with a substituent group, a size-limited substituent group, or lower substituent group) or unsubstituted alkyl (e.g., C₁-C₈, C₁-C₆, or C₁-C₄ alkyl), substituted (e.g., substituted with a substituent group, a size-limited substituent group, or lower substituent group) or unsubstituted heteroalkyl (e.g., 2 to 8 membered, 2 to 6 membered, or 2 to 4 membered heteroalkyl), substituted (e.g., substituted with a substituent group, a size-limited substituent group, or lower substituent group) or unsubstituted cycloalkyl (e.g., C₃-C₈, C₃-C₆, or C₅-C₆ cycloalkyl), substituted (e.g., substituted with a substituent group, a size-limited substituent group, or lower substituent group) or unsubstituted heterocycloalkyl (e.g., 3 to 8 membered, 3 to 6 membered, or 5 to 6 membered heterocycloalkyl), substituted (e.g., substituted with a substituent group, a size-limited substituent group, or lower substituent group) or unsubstituted aryl (e.g., C₆-C₁₀, C₁₀ aryl, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered heteroaryl).

In embodiments, a substituted R^{1.1} is substituted with at least one substituent group, size-limited substituent group, or lower substituent group; wherein if the substituted R^{1.1} is substituted with a plurality of groups selected from substituent groups, size-limited substituent groups, and lower substituent groups; each substituent group, size-limited substituent group, and/or lower substituent group may optionally be different. In embodiments, when R^{1.1} is substituted, it is substituted with at least one substituent group. In embodiments, when R^{1.1} is substituted, it is substituted with at least one size-limited substituent group. In embodiments, when R^{1.1} is substituted, it is substituted with at least one lower substituent group.

In embodiments, a substituted R^{1.2} is substituted with at least one substituent group, size-limited substituent group, or lower substituent group; wherein if the substituted R^{1.2} is substituted with a plurality of groups selected from substituent groups, size-limited substituent groups, and lower substituent groups; each substituent group, size-limited substituent group, and/or lower substituent group may optionally be different. In embodiments, when R^{1.2} is substituted, it is substituted with at least one substituent group. In embodiments, when R^{1.2} is substituted, it is substituted with at least one size-limited substituent group. In embodiments, when R^{1.2} is substituted, it is substituted with at least one lower substituent group.

In embodiments, a substituted R^{1.3} is substituted with at least one substituent group, size-limited substituent group, or lower substituent group; wherein if the substituted R^{1.3} is substituted with a plurality of groups selected from substituent groups, size-limited substituent groups, and lower substituent groups; each substituent group, size-limited substituent group, and/or lower substituent group may optionally be different. In embodiments, when R^{1.3} is substituted, it is substituted with at least one substituent group. In embodiments, when R^{1.3} is substituted, it is substituted with at least one size-limited substituent group. In embodiments, when R^{1.3} is substituted, it is substituted with at least one lower substituent group.

In embodiments, a substituted R^{1.4} is substituted with at least one substituent group, size-limited substituent group, or lower substituent group; wherein if the substituted R^{1.4} is substituted with a plurality of groups selected from substituent groups, size-limited substituent groups, and lower substituent groups; each substituent group, size-limited substituent group, and/or lower substituent group may optionally be different. In embodiments, when R^{1.4} is substituted, it is substituted with at least one substituent group. In embodiments, when R^{1.4} is substituted, it is substituted with at least one size-limited substituent group. In embodiments, when R^{1.4} is substituted, it is substituted with at least one lower substituent group.

In embodiments, a substituted R^{1.5} is substituted with at least one substituent group, size-limited substituent group, or lower substituent group; wherein if the substituted R^{1.5} is substituted with a plurality of groups selected from substituent groups, size-limited substituent groups, and lower substituent groups; each substituent group, size-limited substituent group, and/or lower substituent group may optionally be different. In embodiments, when R^{1.5} is substituted, it is substituted with at least one substituent group. In embodiments, when R^{1.5} is substituted, it is substituted with at least one size-limited substituent group. In embodiments, when R^{1.5} is substituted, it is substituted with at least one lower substituent group.

In embodiments, each R¹, R^{1.2}, R^{1.3}, R^{1.4}, and R^{1.5} is independently hydrogen, —F, —C₁, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{1E}-substituted or unsubstituted alkyl (e.g., C₁-C₂₀, C₁-C₁₂, C₁-C₈, C₁-C₆, C₁-C₄, or C₁-C₂), R^{1E}-substituted or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), R^{1E}-substituted or unsubstituted cycloalkyl (e.g., C₃-C₁₀, C₃-C₈, C₃-C₆, C₄-C₆, or C₅-C₆), R^{1E}-substituted or unsubstituted heterocycloalkyl (e.g., 3 to 10 membered, 3 to 8 membered, 3 to 6 membered, 4 to 6 membered, 4 to 5 membered, or 5 to 6 membered), R^{1E}-substituted or unsubstituted aryl (e.g., C₆-C₁₂, C₆-C₁₀, or phenyl), or R^{1E}-substituted or unsubstituted heteroaryl (e.g., 5 to 12 membered, 5 to 10 membered, 5 to 9 membered, or 5 to 6 membered). In embodiments, each R^{1.1}, R^{1.2}, R^{1.3}, R^{1.4}, and R^{1.5} is independently hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{1E}-substituted alkyl (e.g., C₁-C₂₀, C₁-C₁₂, C₁-C₈, C₁-C₆, C₁-C₄, or C₁-C₂), R^{1E}-substituted heteroalkyl (e.g., 2 to 20 membered, 2 to 12 membered, 2 to 8 membered, 2 to 6 membered, 4 to 6 membered, 2 to 3 membered, or 4 to 5 membered), R^{1E}-substituted cycloalkyl (e.g., C₃-C₁₀, C₃-C₈, C₃-C₆, C₄-C₆, or C₅-C₆), R^{1E}-substituted heterocycloalkyl (e.g., 3 to 10 membered, 3 to 8 membered, 3 to 6 membered, 4 to 6 membered, 4 to 5 membered, or 5 to 6 membered), R^{1E}-substituted aryl (e.g., C₆-C₁₂, C₆-C₁₀, or phenyl), or R^{1E}-substituted heteroaryl (e.g., 5 to 12 membered, 5 to 10 membered, 5 to 9 membered, or 5 to 6 membered). In embodiments, each R¹, R^{1.2}, R^{1.3}, R^{1.4}, and R^{1.5} is independently hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

R^{1E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{1F}-substituted or unsubstituted alkyl (e.g., C₁-C₈, C₁-C₆, or C₁-C₄ alkyl), R^{1F}-substituted or unsubstituted heteroalkyl (e.g., 2 to 8 membered, 2 to 6 membered, or 2 to 4 membered heteroalkyl), R^{1F}-substituted or unsubstituted cycloalkyl (e.g., C₃-C₈, C₃-C₆, or C₅-C₆ cycloalkyl), R^{1F}-substituted or unsubstituted heterocycloalkyl (e.g., 3 to 8 membered, 3 to 6 membered, or 5 to 6 membered heterocycloalkyl), R^{1F}-substituted or unsubstituted aryl (e.g., C₆-C₁₀, C₁₀ aryl, or phenyl), or R^{1F}-substituted or unsubstituted heteroaryl (e.g., 5 to 10 membered, 5 to 9 membered, or 5 to 6 membered heteroaryl). In embodiments, R^{1E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{1F}-substituted alkyl (e.g., C₁-C₈, C₁-C₆, or C₁-C₄ alkyl), R^{1F}-substituted heteroalkyl (e.g., 2 to 8 membered, 2 to 6 membered, or 2 to 4 membered heteroalkyl), R^{1F}-substituted cycloalkyl (e.g., C₃-C₈, C₃-C₆, or C₅-C₆ cycloalkyl), R^{1F}-substituted heterocycloalkyl (e.g., 3 to 8 membered, 3 to 6 membered, or 5 to 6 membered heterocycloalkyl), R^{1F}-substituted aryl (e.g., C₆-C₁₀, C₁₀ aryl, or phenyl), or R^{1F}-substituted heteroaryl (e.g., 5 to 10 membered, 5 to 9 membered, or 5 to 6 membered heteroaryl). In embodiments, R^{1E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

R¹⁶ is halogen (e.g., —F, —Cl, —Br, or —I), —CX¹⁶₃ (e.g., —CF₃, —CCl₃, —CBr₃, or —C₃), —CHX¹⁶₂ (e.g., —CHF₂, —CHCl₂, —CHBr₂, or —CHI₂), —CH₂X¹⁶ (e.g., —CH₂F, —CH₂Cl, —CH₂Br, or —CH₂I), —OCX¹⁶₃ (e.g., —OCF₃, —OCCl₃, —OCBr₃, or —OCI₃), —OCH₂X¹⁶ (e.g., —OCH₂F, —OCH₂Cl, —OCH₂Br, or —OCH₂I), —OCHX¹⁶₂ (e.g., —OCHF₂, —OCHCl₂, —OCHBr₂, —OCHI₂), —CN, —SOₙ₁₆R^{16D} (e.g., —SH, —SCH₃, —SO₂H, —SO₃H, or —SO₄H), —SOᵥ₁₆NR^{16A}R^{16B}(e.g., —SO₂NH₂, or —SO₂NHCH₃), —NR^{16C}NR^{16A}R^{16B} (e.g., NHNH₂ or NHNHCH₃), —ONR^{16A}R^{16B} (e.g., —ONH₂, or —ONHCH₃), —NHC(O)NR^{16C}NR^{16A}R^{16B} (e.g., —NHC(O)NHNH₂, or —NHC(O)NHNHCH₃), —NHC(O)NR^{16A}R¹⁶ (e.g., —NHC(O)NH₂, or —NHC(O)NHCH₃), —N(O)ₘ₁₆ (e.g., —NO, or —NO₂), —NR^{16A}R^{16B} (e.g., —NH₂, or —NHCH₃), —C(O)R^{16C} (e.g., —C(O)H or —C(O)CH₃), —C(O)—OR^{16C} (e.g., —C(O)OH or —C(O)OCH₃), —C(O)NR^{16A}R^{16B} (e.g., —C(O)NH₂ or —C(O) NHCH₃), —OR^{16D} (e.g., —OH, or —OCH₃), —NR^{16A}SO₂R^{16D} (e.g., —NHSO₂H), —NR^{16A}C(O)R^{16C} (e.g., —NHCOH), —NR^{16A}C(O)OR^{16C} (e.g., —NHC(O)OH), —NR^{16A}OR^{16C} (e.g., —NHOH), —N₃, substituted (e.g., substituted with a substituent group, a size-limited substituent group, or lower substituent group) or unsubstituted alkyl (e.g., C₁-C₈, C₁-C₆, or C₁-C₄ alkyl), substituted (e.g., substituted with a substituent group, a size-limited substituent group, or lower substituent group) or unsubstituted heteroalkyl (e.g., 2 to 8 membered, 2 to 6 membered, or 2 to 4 membered heteroalkyl), substituted (e.g., substituted with a substituent group, a size-limited substituent group, or lower substituent group) or unsubstituted cycloalkyl (e.g., C₃-C₈, C₃-C₆, or C₅-C₆ cycloalkyl), substituted (e.g., substituted with a substituent group, a size-limited substituent group, or lower substituent group) or unsubstituted heterocycloalkyl (e.g., 3 to 8 membered, 3 to 6 membered, or 5 to 6 membered heterocycloalkyl), substituted (e.g., substituted with a substituent group, a size-limited substituent group, or lower substituent group) or unsubstituted aryl (e.g., C₆-C₁₀, C₁₀ aryl, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered heteroaryl).

In embodiments, a substituted R¹⁶ is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, R¹⁶ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{16E}-substituted or unsubstituted alkyl (e.g., C₁-C₂₀, C₁-C₁₂, C₁-C₈, C₁-C₆, C₁-C₄, or C₁-C₂), R^{16E}-substituted or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), R^{16E}-substituted or unsubstituted cycloalkyl (e.g., C₃-C₁₀, C₃-C₈, C₃-C₆, C₄-C₆, or C₅-C₆), R^{16E}-substituted or unsubstituted heterocycloalkyl (e.g., 3 to 10 membered, 3 to 8 membered, 3 to 6 membered, 4 to 6 membered, 4 to 5 membered, or 5 to 6 membered), R^{16E}-substituted or unsubstituted aryl (e.g., C₆-C₁₂, C₆-C₁₀, or phenyl), or R^{16E}-substituted or unsubstituted heteroaryl (e.g., 5 to 12 membered, 5 to 10 membered, 5 to 9 membered, or 5 to 6 membered). In embodiments, R¹⁶ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{16E}-substituted alkyl (e.g., C₁-C₂₀, C₁-C₁₂, C₁-C₈, C₁-C₆, C₁-C₄, or C₁-C₂), R^{16E}-substituted heteroalkyl (e.g., 2 to 20 membered, 2 to 12 membered, 2 to 8 membered, 2 to 6 membered, 4 to 6 membered, 2 to 3 membered, or 4 to 5 membered), R^{16E}-substituted cycloalkyl (e.g., C₃-C₁₀, C₃-C₈, C₃-C₆, C₄-C₆, or C₅-C₆), R^{16E}-substituted heterocycloalkyl (e.g., 3 to 10 membered, 3 to 8 membered, 3 to 6 membered, 4 to 6 membered, 4 to 5 membered, or 5 to 6 membered), R^{16E}-substituted aryl (e.g., C₆-C₁₂, C₆-C₁₀, or phenyl), or R^{16E}-substituted heteroaryl (e.g., 5 to 12 membered, 5 to 10 membered, 5 to 9 membered, or 5 to 6 membered). In embodiments, R¹⁶ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

R^{16E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OC₃, —OCHF₂, —OCHCl₂, —OCHBr₂, —OCHI₂, —OCH₂F, —OCH₂Cl, —OCH₂Br, —OCH₂I, R^{16F}-substituted or unsubstituted alkyl (e.g., C₁-C₈, C₁-C₆, or C₁-C₄ alkyl), R^{16F}-substituted or unsubstituted heteroalkyl (e.g., 2 to 8 membered, 2 to 6 membered, or 2 to 4 membered heteroalkyl), R^{16F}-substituted or unsubstituted cycloalkyl (e.g., C₃-C₈, C₃-C₆, or C₅-C₆ cycloalkyl), R^{16F}-substituted or unsubstituted heterocycloalkyl (e.g., 3 to 8 membered, 3 to 6 membered, or 5 to 6 membered heterocycloalkyl), R^{16F}-substituted or unsubstituted aryl (e.g., C₆-C₁₀, C₁₀ aryl, or phenyl), or R^{16F}-substituted or unsubstituted heteroaryl (e.g., 5 to 10 membered, 5 to 9 membered, or 5 to 6 membered heteroaryl). In embodiments, R^{16E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{16F}-substituted alkyl (e.g., C₁-C₈, C₁-C₆, or C₁-C₄ alkyl), R^{16F}-substituted heteroalkyl (e.g., 2 to 8 membered, 2 to 6 membered, or 2 to 4 membered heteroalkyl), R^{16F}-substituted cycloalkyl (e.g., C₃-C₈, C₃-C₆, or C₅-C₆ cycloalkyl), R^{16F}-substituted heterocycloalkyl (e.g., 3 to 8 membered, 3 to 6 membered, or 5 to 6 membered heterocycloalkyl), R^{16F}-substituted aryl (e.g., C₆-C₁₀, C₁₀ aryl, or phenyl), or R^{16F}-substituted heteroaryl (e.g., 5 to 10 membered, 5 to 9 membered, or 5 to 6 membered heteroaryl). In embodiments, R^{16E} is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

R¹⁷ is halogen (e.g., —F, —Cl, —Br, or —I), —CX¹⁷₃ (e.g., —CF₃, —CCl₃, —CBr₃, or —C₃), —CHX¹⁷₂ (e.g., —CHF₂, —CHCl₂, —CHBr₂, or —CHI₂), —CH₂X¹⁷ (e.g., —CH₂F, —CH₂Cl, —CH₂Br, or —CH₂I), —OCX¹⁷₃ (e.g., —OCF₃, —OCCl₃, —OCBr₃, or —OCI₃), —OCH₂X¹⁷ (e.g., —OCH₂F, —OCH₂Cl, —OCH₂Br, or —OCH₂I), —OCHX¹⁷₂ (e.g., —OCHF₂, —OCHCl₂, —OCHBr₂, —OCHI₂), —CN, —SOₙ₁₇R^{17D} (e.g., —SH, —SCH₃, —SO₂H, —SO₃H, or —SO₄H), —SOₙ₁₇NR^{17A}R^{17B}(e.g., —SO₂NH₂, or —SO₂NHCH₃), —NR^{17C}NR^{17A}R^{17B} (e.g., NHNH₂ or NHNHCH₃), —ONR^{17A}R^{17B} (e.g., —ONH₂, or —ONHCH₃), —NHC(O)NR^{17C}NR^{17A}R^{17B} (e.g., —NHC(O)NHNH₂, or —NHC(O)NHNHCH₃), —NHC(O)NR^{17A}R^{17B} (e.g., —NHC(O)NH₂, or —NHC(O)NHCH₃), —N(O)ₘ₁₇ (e.g., —NO, or —NO₂), —NR^{17A}R^{17B} (e.g., —NH₂, or —NHCH₃), —C(O)R^{17C} (e.g., —C(O)H or —C(O)CH₃), —C(O)—OR^{17C} (e.g., —C(O)OH or —C(O)OCH₃), —C(O)NR^{17A}R^{17B} (e.g., —C(O)NH₂ or —C(O) NHCH₃), —OR^{17D} (e.g., —OH, or —OCH₃), —NR^{17A}S02R^{17D} (e.g., —NHSO₂H), —NR^{17A}C(O)R^{17C} (e.g., —NHCOH), —NR^{17A}C(O)OR^{17C} (e.g., —NHC(O)OH), —NR^{17A}OR^{17C} (e.g., —NHOH), —N₃, substituted (e.g., substituted with a substituent group, a size-limited substituent group, or lower substituent group) or unsubstituted alkyl (e.g., C₁-C₈, C₁-C₆, or C₁-C₄ alkyl), substituted (e.g., substituted with a substituent group, a size-limited substituent group, or lower substituent group) or unsubstituted heteroalkyl (e.g., 2 to 8 membered, 2 to 6 membered, or 2 to 4 membered heteroalkyl), substituted (e.g., substituted with a substituent group, a size-limited substituent group, or lower substituent group) or unsubstituted cycloalkyl (e.g., C₃-C₈, C₃-C₆, or C₅-C₆ cycloalkyl), substituted (e.g., substituted with a substituent group, a size-limited substituent group, or lower substituent group) or unsubstituted heterocycloalkyl (e.g., 3 to 8 membered, 3 to 6 membered, or 5 to 6 membered heterocycloalkyl), substituted (e.g., substituted with a substituent group, a size-limited substituent group, or lower substituent group) or unsubstituted aryl (e.g., C₆-C₁₀, C₁₀ aryl, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered heteroaryl).

In embodiments, a substituted R¹⁷ is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁷ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{17E}-substituted or unsubstituted alkyl (e.g., C₁-C₂₀, C₁-C₁₂, C₁-C₈, C₁-C₆, C₁-C₄, or C₁-C₂), R^{17E}-substituted or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), R^{17E}-substituted or unsubstituted cycloalkyl (e.g., C₃-C₁₀, C₃-C₈, C₃-C₆, C₄-C₆, or C₅-C₆), R^{17E}-substituted or unsubstituted heterocycloalkyl (e.g., 3 to 10 membered, 3 to 8 membered, 3 to 6 membered, 4 to 6 membered, 4 to 5 membered, or 5 to 6 membered), R^{17E}-substituted or unsubstituted aryl (e.g., C₆-C₁₂, C₆-C₁₀, or phenyl), or R^{17E}-substituted or unsubstituted heteroaryl (e.g., 5 to 12 membered, 5 to 10 membered, 5 to 9 membered, or 5 to 6 membered). In embodiments, R¹⁷ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{17E}-substituted alkyl (e.g., C₁-C₂₀, C₁-C₁₂, C₁-C₈, C₁-C₆, C₁-C₄, or C₁-C₂), R^{17E}-substituted heteroalkyl (e.g., 2 to 20 membered, 2 to 12 membered, 2 to 8 membered, 2 to 6 membered, 4 to 6 membered, 2 to 3 membered, or 4 to 5 membered), R^{17E}-substituted cycloalkyl (e.g., C₃-C₁₀, C₃-C₈, C₃-C₆, C₄-C₆, or C₅-C₆), R^{17E}-substituted heterocycloalkyl (e.g., 3 to 10 membered, 3 to 8 membered, 3 to 6 membered, 4 to 6 membered, 4 to 5 membered, or 5 to 6 membered), R^{17E}-substituted aryl (e.g., C₆-C₁₂, C₆-C₁₀, or phenyl), or R^{17E}-substituted heteroaryl (e.g., 5 to 12 membered, 5 to 10 membered, 5 to 9 membered, or 5 to 6 membered). In embodiments, R¹⁷ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

R^{17E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{17F}-substituted or unsubstituted alkyl (e.g., C₁-C₈, C₁-C₆, or C₁-C₄ alkyl), R^{17F}-substituted or unsubstituted heteroalkyl (e.g., 2 to 8 membered, 2 to 6 membered, or 2 to 4 membered heteroalkyl), R^{17F}-substituted or unsubstituted cycloalkyl (e.g., C₃-C₈, C₃-C₆, or C₅-C₆ cycloalkyl), R^{17F}-substituted or unsubstituted heterocycloalkyl (e.g., 3 to 8 membered, 3 to 6 membered, or 5 to 6 membered heterocycloalkyl), R^{17F}-substituted or unsubstituted aryl (e.g., C₆-C₁₀, C₁₀ aryl, or phenyl), or R^{17F}-substituted or unsubstituted heteroaryl (e.g., 5 to 10 membered, 5 to 9 membered, or 5 to 6 membered heteroaryl). In embodiments, R^{17E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{17F}-substituted alkyl (e.g., C₁-C₈, C₁-C₆, or C₁-C₄ alkyl), R^{17F}-substituted heteroalkyl (e.g., 2 to 8 membered, 2 to 6 membered, or 2 to 4 membered heteroalkyl), R^{17F}-substituted cycloalkyl (e.g., C₃-C₈, C₃-C₆, or C₅-C₆ cycloalkyl), R^{17F}-substituted heterocycloalkyl (e.g., 3 to 8 membered, 3 to 6 membered, or 5 to 6 membered heterocycloalkyl), R^{17F}-substituted aryl (e.g., C₆-C₁₀, C₁₀ aryl, or phenyl), or R^{17F}-substituted heteroaryl (e.g., 5 to 10 membered, 5 to 9 membered, or 5 to 6 membered heteroaryl). In embodiments, R^{17E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

R¹⁸ is halogen (e.g., —F, —Cl, —Br, or —I), —CX¹⁸₃ (e.g., —CF₃, —CCl₃, —CBr₃, or —C₃), —CHX¹⁸₂ (e.g., —CHF₂, —CHCl₂, —CHBr₂, or —CHI₂), —CH₂X¹⁸ (e.g., —CH₂F, —CH₂Cl, —CH₂Br, or —CH₂I), —OCX¹⁸₃ (e.g., —OCF₃, —OCCl₃, —OCBr₃, or —OCI₃), —OCH₂X¹⁸ (e.g., —OCH₂F, —OCH₂Cl, —OCH₂Br, or —OCH₂I), —OCHX¹⁸₂ (e.g., —OCHF₂, —OCHCl₂, —OCHBr₂, —OCHI₂), —CN, —SOₙ₁₈R^{18D} (e.g., —SH, —SCH₃, —SO₂H, —SO₃H, or —SO₄H), —SOᵥ₁₈NR^{18A}R^{18B} (e.g., —SO₂NH₂, or —SO₂NHCH₃), —NR^{18C}NR^{18A}R^{18B} (e.g., NHNH₂ or NHNHCH₃), —ONR^{18A}R^{18B} (e.g., —ONH₂, or —ONHCH₃), —NHC(O)NR^{18C}NR^{18A}R^{18B} (e.g., —NHC(O)NHNH₂, or —NHC(O)NHNHCH₃), —NHC(O)NR^{18A}R^{18B} (e.g., —NHC(O)NH₂, or —NHC(O)NHCH₃), —N(O)ₘ₁₈ (e.g., —NO, or —NO₂), —NR^{18A}R^{18B} (e.g., —NH₂, or —NHCH₃), —C(O)R^{18C} (e.g., —C(O)H or —C(O)CH₃), —C(O)—OR^{18C} (e.g., —C(O)OH or —C(O)OCH₃), —C(O)NR^{18A}R^{18B} (e.g., —C(O)NH₂ or —C(O) NHCH₃), —OR^{18D} (e.g., —OH, or —OCH₃), —NR^{18A}SO₂R^{18D} (e.g., —NHSO₂H), —NR^{18A}C(O)R^{18C} (e.g., —NHCOH), —NR^{18A}C(O)OR^{18C} (e.g., —NHC(O)OH), —NR^{18A}OR^{18C} (e.g., —NHOH), —N₃, substituted (e.g., substituted with a substituent group, a size-limited substituent group, or lower substituent group) or unsubstituted alkyl (e.g., C₁-C₈, C₁-C₆, or C₁-C₄ alkyl), substituted (e.g., substituted with a substituent group, a size-limited substituent group, or lower substituent group) or unsubstituted heteroalkyl (e.g., 2 to 8 membered, 2 to 6 membered, or 2 to 4 membered heteroalkyl), substituted (e.g., substituted with a substituent group, a size-limited substituent group, or lower substituent group) or unsubstituted cycloalkyl (e.g., C₃-C₈, C₃-C₆, or C₅-C₆ cycloalkyl), substituted (e.g., substituted with a substituent group, a size-limited substituent group, or lower substituent group) or unsubstituted heterocycloalkyl (e.g., 3 to 8 membered, 3 to 6 membered, or 5 to 6 membered heterocycloalkyl), substituted (e.g., substituted with a substituent group, a size-limited substituent group, or lower substituent group) or unsubstituted aryl (e.g., C₆-C₁₀, C₁₀ aryl, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered heteroaryl).

In embodiments, a substituted R¹⁸ is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

In embodiments, R¹⁸ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{18E}-substituted or unsubstituted alkyl (e.g., C₁-C₂₀, C₁-C₁₂, C₁-C₈, C₁-C₆, C₁-C₄, or C₁-C₂), R^{18E}-substituted or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), R^{18E}-substituted or unsubstituted cycloalkyl (e.g., C₃-C₁₀, C₃-C₈, C₃-C₆, C₄-C₆, or C₅-C₆), R^{18E}-substituted or unsubstituted heterocycloalkyl (e.g., 3 to 10 membered, 3 to 8 membered, 3 to 6 membered, 4 to 6 membered, 4 to 5 membered, or 5 to 6 membered), R^{18E}-substituted or unsubstituted aryl (e.g., C₆-C₁₂, C₆-C₁₀, or phenyl), or R^{18E}-substituted or unsubstituted heteroaryl (e.g., 5 to 12 membered, 5 to 10 membered, 5 to 9 membered, or 5 to 6 membered). In embodiments, R¹⁸ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{18E}-substituted alkyl (e.g., C₁-C₂₀, C₁-C₁₂, C₁-C₈, C₁-C₆, C₁-C₄, or C₁-C₂), R^{18E}-substituted heteroalkyl (e.g., 2 to 20 membered, 2 to 12 membered, 2 to 8 membered, 2 to 6 membered, 4 to 6 membered, 2 to 3 membered, or 4 to 5 membered), R^{18E}-substituted cycloalkyl (e.g., C₃-C₁₀, C₃-C₈, C₃-C₆, C₄-C₆, or C₅-C₆), R^{18E}-substituted heterocycloalkyl (e.g., 3 to 10 membered, 3 to 8 membered, 3 to 6 membered, 4 to 6 membered, 4 to 5 membered, or 5 to 6 membered), R^{18E}-substituted aryl (e.g., C₆-C₁₂, C₆-C₁₀, or phenyl), or R^{18E}-substituted heteroaryl (e.g., 5 to 12 membered, 5 to 10 membered, 5 to 9 membered, or 5 to 6 membered). In embodiments, R¹⁸ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

R^{18E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{18F}-substituted or unsubstituted alkyl (e.g., C₁-C₈, C₁-C₆, or C₁-C₄ alkyl), R^{18F}-substituted or unsubstituted heteroalkyl (e.g., 2 to 8 membered, 2 to 6 membered, or 2 to 4 membered heteroalkyl), R^{18F}-substituted or unsubstituted cycloalkyl (e.g., C₃-C₈, C₃-C₆, or C₅-C₆ cycloalkyl), R^{18F}-substituted or unsubstituted heterocycloalkyl (e.g., 3 to 8 membered, 3 to 6 membered, or 5 to 6 membered heterocycloalkyl), R^{18F}-substituted or unsubstituted aryl (e.g., C₆-C₁₀, C₁₀ aryl, or phenyl), or R^{18F}-substituted or unsubstituted heteroaryl (e.g., 5 to 10 membered, 5 to 9 membered, or 5 to 6 membered heteroaryl). In embodiments, R^{18E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{18F}-substituted alkyl (e.g., C₁-C₈, C₁-C₆, or C₁-C₄ alkyl), R^{18F}-substituted heteroalkyl (e.g., 2 to 8 membered, 2 to 6 membered, or 2 to 4 membered heteroalkyl), R^{18F}-substituted cycloalkyl (e.g., C₃-C₈, C₃-C₆, or C₅-C₆ cycloalkyl), R^{18F}-substituted heterocycloalkyl (e.g., 3 to 8 membered, 3 to 6 membered, or 5 to 6 membered heterocycloalkyl), R^{18F}-substituted aryl (e.g., C₆-C₁₀, C₁₀ aryl, or phenyl), or R^{18F}-substituted heteroaryl (e.g., 5 to 10 membered, 5 to 9 membered, or 5 to 6 membered heteroaryl). In embodiments, R^{18E} is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

R¹⁹ is halogen (e.g., —F, —Cl, —Br, or —I), —CX¹⁹₃ (e.g., —CF₃, —CCl₃, —CBr₃, or —CI₃), —CHX¹⁹₂ (e.g., —CHF₂, —CHCl₂, —CHBr₂, or —CHI₂), —CH₂X¹⁹ (e.g., —CH₂F, —CH₂Cl, —CH₂Br, or —CH₂I), —OCX¹⁹₃ (e.g., —OCF₃, —OCCl₃, —OCBr₃, or —OCI₃), —OCH₂X¹⁹ (e.g., —OCH₂F, —OCH₂Cl, —OCH₂Br, or —OCH₂I), —OCHX¹⁹₂ (e.g., —OCHF₂, —OCHCl₂, —OCHBr₂, —OCHI₂), —CN, —SOₙ₁₉R^{19D} (e.g., —SH, —SCH₃, —SO₂H, —SO₃H, or —SO₄H), —SOᵥ₁₉NR^{19A}R^{19B}(e.g., —SO₂NH₂, or —SO₂NHCH₃), —NR^{19C}NR^{19A}R^{19B} (e.g., NHNH₂ or NHNHCH₃), —ONR^{19A}R^{19B} (e.g., —ONH₂, or —ONHCH₃), —NHC(O)NR^{19C}NR^{19A}R^{19B} (e.g., —NHC(O)NHNH₂, or —NHC(O)NHNHCH₃), —NHC(O)NR^{19A}R^{19B} (e.g., —NHC(O)NH₂, or —NHC(O)NHCH₃), —N(O)ₘ₁₉ (e.g., —NO, or —NO₂), —NR^{19A}R^{19B} (e.g., —NH₂, or —NHCH₃), —C(O)R^{19C} (e.g., —C(O)H or —C(O)CH₃), —C(O)—OR^{19C} (e.g., —C(O)OH or —C(O)OCH₃), —C(O)NR^{19A}R^{19B} (e.g., —C(O)NH₂ or —C(O) NHCH₃), —OR^{19D} (e.g., —OH, or —OCH₃), —NR^{19A}SO₂R^{19D} (e.g., —NHSO₂H), —NR^{19A}C(O)R^{19C} (e.g., —NHCOH), —NR^{19A}C(O)OR^{19C} (e.g., —NHC(O)OH), —NR^{19A}OR^{19C} (e.g., —NHOH), —N₃, substituted (e.g., substituted with a substituent group, a size-limited substituent group, or lower substituent group) or unsubstituted alkyl (e.g., C₁-C₈, C₁-C₆, or C₁-C₄ alkyl), substituted (e.g., substituted with a substituent group, a size-limited substituent group, or lower substituent group) or unsubstituted heteroalkyl (e.g., 2 to 8 membered, 2 to 6 membered, or 2 to 4 membered heteroalkyl), substituted (e.g., substituted with a substituent group, a size-limited substituent group, or lower substituent group) or unsubstituted cycloalkyl (e.g., C₃-C₈, C₃-C₆, or C₅-C₆ cycloalkyl), substituted (e.g., substituted with a substituent group, a size-limited substituent group, or lower substituent group) or unsubstituted heterocycloalkyl (e.g., 3 to 8 membered, 3 to 6 membered, or 5 to 6 membered heterocycloalkyl), substituted (e.g., substituted with a substituent group, a size-limited substituent group, or lower substituent group) or unsubstituted aryl (e.g., C₆-C₁₀, C₁₀ aryl, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered heteroaryl).

In embodiments, a substituted R¹⁹ is substituted with at least one substituent group, size-limited substituent group, or lower substituent group; wherein if the substituted R¹⁹ is substituted with a plurality of groups selected from substituent groups, size-limited substituent groups, and lower substituent groups; each substituent group, size-limited substituent group, and/or lower substituent group may optionally be different. In embodiments, when R¹⁹ is substituted, it is substituted with at least one substituent group. In embodiments, when R¹⁹ is substituted, it is substituted with at least one size-limited substituent group. In embodiments, when R¹⁹ is substituted, it is substituted with at least one lower substituent group.

In embodiments, R¹⁹ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{19E}-substituted or unsubstituted alkyl (e.g., C₁-C₂₀, C₁-C₁₂, C₁-C₈, C₁-C₆, C₁-C₄, or C₁-C₂), R^{19E}-substituted or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), R^{19E}-substituted or unsubstituted cycloalkyl (e.g., C₃-C₁₀, C₃-C₈, C₃-C₆, C₄-C₆, or C₅-C₆), R^{19E}-substituted or unsubstituted heterocycloalkyl (e.g., 3 to 10 membered, 3 to 8 membered, 3 to 6 membered, 4 to 6 membered, 4 to 5 membered, or 5 to 6 membered), R^{19E}-substituted or unsubstituted aryl (e.g., C₆-C₁₂, C₆-C₁₀, or phenyl), or R^{19E}-substituted or unsubstituted heteroaryl (e.g., 5 to 12 membered, 5 to 10 membered, 5 to 9 membered, or 5 to 6 membered). In embodiments, R¹⁹ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{19E}-substituted alkyl (e.g., C₁-C₂₀, C₁-C₁₂, C₁-C₈, C₁-C₆, C₁-C₄, or C₁-C₂), R^{19E}-substituted heteroalkyl (e.g., 2 to 20 membered, 2 to 12 membered, 2 to 8 membered, 2 to 6 membered, 4 to 6 membered, 2 to 3 membered, or 4 to 5 membered), R^{19E}-substituted cycloalkyl (e.g., C₃-C₁₀, C₃-C₈, C₃-C₆, C₄-C₆, or C₅-C₆), R^{19E}-substituted heterocycloalkyl (e.g., 3 to 10 membered, 3 to 8 membered, 3 to 6 membered, 4 to 6 membered, 4 to 5 membered, or 5 to 6 membered), R^{19E}-substituted aryl (e.g., C₆-C₁₂, C₆-C₁₀, or phenyl), or R^{19E}-substituted heteroaryl (e.g., 5 to 12 membered, 5 to 10 membered, 5 to 9 membered, or 5 to 6 membered). In embodiments, R¹⁹ is hydrogen, —F, —Cl, —Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

R^{19E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{19F}-substituted or unsubstituted alkyl (e.g., C₁-C₈, C₁-C₆, or C₁-C₄ alkyl), R^{19F}-substituted or unsubstituted heteroalkyl (e.g., 2 to 8 membered, 2 to 6 membered, or 2 to 4 membered heteroalkyl), R^{19F}-substituted or unsubstituted cycloalkyl (e.g., C₃-C₈, C₃-C₆, or C₅-C₆ cycloalkyl), R^{19F}-substituted or unsubstituted heterocycloalkyl (e.g., 3 to 8 membered, 3 to 6 membered, or 5 to 6 membered heterocycloalkyl), R^{19F}-substituted or unsubstituted aryl (e.g., C₆-C₁₀, C₁₀ aryl, or phenyl), or R^{19F}-substituted or unsubstituted heteroaryl (e.g., 5 to 10 membered, 5 to 9 membered, or 5 to 6 membered heteroaryl). In embodiments, R^{19E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{19F}-substituted alkyl (e.g., C₁-C₈, C₁-C₆, or C₁-C₄ alkyl), R^{19F}-substituted heteroalkyl (e.g., 2 to 8 membered, 2 to 6 membered, or 2 to 4 membered heteroalkyl), R^{19F}-substituted cycloalkyl (e.g., C₃-C₈, C₃-C₆, or C₅-C₆ cycloalkyl), R^{19F}-substituted heterocycloalkyl (e.g., 3 to 8 membered, 3 to 6 membered, or 5 to 6 membered heterocycloalkyl), R^{19F}-substituted aryl (e.g., C₆-C₁₀, C₁₀ aryl, or phenyl), or R^{19F}-substituted heteroaryl (e.g., 5 to 10 membered, 5 to 9 membered, or 5 to 6 membered heteroaryl). In embodiments, R^{19E} is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OC₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

L¹ is a

bond, —NH—, —O—, —S—, —C(O)—, —C(O)NH—, —NHC(O)—, —NHC(O)NH—, —C(O)O—, —OC(O)—, substituted (e.g., substituted with a substituent group, a size-limited substituent group, or lower substituent group) or unsubstituted alkylene (e.g., C₁-C₈, C₁-C₆, or C₁-C₄ alkylene), substituted (e.g., substituted with a substituent group, a size-limited substituent group, or lower substituent group) or unsubstituted heteroalkylene (e.g., 2 to 8 membered, 2 to 6 membered, or 2 to 4 membered heteroalkylene), substituted (e.g., substituted with a substituent group, a size-limited substituent group, or lower substituent group) or unsubstituted cycloalkylene (e.g., C₃-C₈, C₃-C₆, or C₅-C₆ cycloalkylene), substituted (e.g., substituted with a substituent group, a size-limited substituent group, or lower substituent group) or unsubstituted heterocycloalkylene (e.g., 3 to 8 membered, 3 to 6 membered, or 5 to 6 membered heterocycloalkylene), substituted (e.g., substituted with a substituent group, a size-limited substituent group, or lower substituent group) or unsubstituted arylene (e.g., C₆-C₁₀, C₁₀ aryl, or phenylene), or substituted (e.g., substituted with a substituent group, a size-limited substituent group, or lower substituent group) or unsubstituted heteroarylene (e.g., 5 to 10 membered, 5 to 9 membered, or 5 to 6 membered heteroarylene).

In embodiments, a substituted L¹ is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a

bond, —NH—, —O—, —S—, —C(O)—, —C(O)NH—, —NHC(O)—, —NHC(O)NH—, —C(O)O—, —OC(O)—, R²⁰-substituted or unsubstituted alkylene (e.g., C₁-C₈, C₁-C₆, or C₁-C₄ alkylene), R²⁰-substituted or unsubstituted heteroalkylene (e.g., 2 to 8 membered, 2 to 6 membered, or 2 to 4 membered heteroalkylene), R²⁰-substituted or unsubstituted cycloalkylene (e.g., C₃-C₈, C₃-C₆, or C₅-C₆ cycloalkylene), R²⁰-substituted or unsubstituted heterocycloalkylene (e.g., 3 to 8 membered, 3 to 6 membered, or 5 to 6 membered heterocycloalkylene), R²⁰-substituted or unsubstituted arylene (e.g., C₆-C₁₀, C₁₀ aryl, or phenylene), or R²⁰-substituted or unsubstituted heteroarylene (e.g., 5 to 10 membered, 5 to 9 membered, or 5 to 6 membered heteroarylene). In embodiments, L¹ is a
bond, —NH—, —O—, —S—, —C(O)—, —C(O)NH—, —NHC(O)—, —NHC(O)NH—, —C(O)O—, —OC(O)—, R²⁰-substituted alkylene (e.g., C₁-C₈, C₁-C₆, or C₁-C₄ alkylene), R²⁰-substituted heteroalkylene (e.g., 2 to 8 membered, 2 to 6 membered, or 2 to 4 membered heteroalkylene), R²⁰-substituted cycloalkylene (e.g., C₃-C₈, C₃-C₆, or C₅-C₆ cycloalkylene), R²⁰-substituted heterocycloalkylene (e.g., 3 to 8 membered, 3 to 6 membered, or 5 to 6 membered heterocycloalkylene), R²⁰-substituted arylene (e.g., C₆-C₁₀, C₁₀ aryl, or phenylene), or R²⁰-substituted heteroarylene (e.g., 5 to 10 membered, 5 to 9 membered, or 5 to 6 membered heteroarylene). In embodiments, L¹ is a
bond, —NH—, —O—, —S—, —C(O)—, —C(O)NH—, —NHC(O)—, —NHC(O)NH—, —C(O)O—, —OC(O)—, unsubstituted alkylene (e.g., C₁-C₈, C₁-C₆, or C₁-C₄ alkylene), unsubstituted heteroalkylene (e.g., 2 to 8 membered, 2 to 6 membered, or 2 to 4 membered heteroalkylene), unsubstituted cycloalkylene (e.g., C₃-C₈, C₃-C₆, or C₅-C₆ cycloalkylene), unsubstituted heterocycloalkylene (e.g., 3 to 8 membered, 3 to 6 membered, or 5 to 6 membered heterocycloalkylene), unsubstituted arylene (e.g., C₆-C₁₀, C₁₀ aryl, or phenylene), or unsubstituted heteroarylene (e.g., 5 to 10 membered, 5 to 9 membered, or 5 to 6 membered heteroarylene).

R²⁰ is oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R²¹-substituted or unsubstituted alkyl (e.g., C₁-C₈, C₁-C₆, or C₁-C₄ alkyl), R²¹-substituted or unsubstituted heteroalkyl (e.g., 2 to 8 membered, 2 to 6 membered, or 2 to 4 membered heteroalkyl), R²¹-substituted or unsubstituted cycloalkyl (e.g., C₃-C₈, C₃-C₆, or C₅-C₆ cycloalkyl), R²¹-substituted or unsubstituted heterocycloalkyl (e.g., 3 to 8 membered, 3 to 6 membered, or 5 to 6 membered heterocycloalkyl), R²¹-substituted or unsubstituted aryl (e.g., C₆-C₁₀, C₁₀ aryl, or phenyl), or R²¹-substituted or unsubstituted heteroaryl (e.g., 5 to 10 membered, 5 to 9 membered, or 5 to 6 membered heteroaryl). In embodiments, R²⁰ is oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R²¹-substituted alkyl (e.g., C₁-C₈, C₁-C₆, or C₁-C₄ alkyl), R²¹-substituted heteroalkyl (e.g., 2 to 8 membered, 2 to 6 membered, or 2 to 4 membered heteroalkyl), R²¹-substituted cycloalkyl (e.g., C₃-C₈, C₃-C₆, or C₅-C₆ cycloalkyl), R²¹-substituted heterocycloalkyl (e.g., 3 to 8 membered, 3 to 6 membered, or 5 to 6 membered heterocycloalkyl), R²¹-substituted aryl (e.g., C₆-C₁₀, C₁₀ aryl, or phenyl), or R²¹-substituted heteroaryl (e.g., 5 to 10 membered, 5 to 9 membered, or 5 to 6 membered heteroaryl). In embodiments, R²⁰ is oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

L² is a

bond, —NH—, —O—, —S—, —C(O)—, —C(O)NH—, —NHC(O)—, —NHC(O)NH—, —C(O)O—, —OC(O)—, substituted (e.g., substituted with a substituent group, a size-limited substituent group, or lower substituent group) or unsubstituted alkylene (e.g., C₁-C₈, C₁-C₆, or C₁-C₄ alkylene), substituted (e.g., substituted with a substituent group, a size-limited substituent group, or lower substituent group) or unsubstituted heteroalkylene (e.g., 2 to 8 membered, 2 to 6 membered, or 2 to 4 membered heteroalkylene), substituted (e.g., substituted with a substituent group, a size-limited substituent group, or lower substituent group) or unsubstituted cycloalkylene (e.g., C₃-C₈, C₃-C₆, or C₅-C₆ cycloalkylene), substituted (e.g., substituted with a substituent group, a size-limited substituent group, or lower substituent group) or unsubstituted heterocycloalkylene (e.g., 3 to 8 membered, 3 to 6 membered, or 5 to 6 membered heterocycloalkylene), substituted (e.g., substituted with a substituent group, a size-limited substituent group, or lower substituent group) or unsubstituted arylene (e.g., C₆-C₁₀, C₁₀ aryl, or phenylene), or substituted (e.g., substituted with a substituent group, a size-limited substituent group, or lower substituent group) or unsubstituted heteroarylene (e.g., 5 to 10 membered, 5 to 9 membered, or 5 to 6 membered heteroarylene).

In embodiments, a substituted L² is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is a

bond, —NH—, —O—, —S—, —C(O)—, —C(O)NH—, —NHC(O)—, —NHC(O)NH—, —C(O)O—, —OC(O)—, R²²-substituted or unsubstituted alkylene (e.g., C₁-C₈, C₁-C₆, or C₁-C₄ alkylene), R²²-substituted or unsubstituted heteroalkylene (e.g., 2 to 8 membered, 2 to 6 membered, or 2 to 4 membered heteroalkylene), R²²-substituted or unsubstituted cycloalkylene (e.g., C₃-C₈, C₃-C₆, or C₅-C₆ cycloalkylene), R²²-substituted or unsubstituted heterocycloalkylene (e.g., 3 to 8 membered, 3 to 6 membered, or 5 to 6 membered heterocycloalkylene), R²²-substituted or unsubstituted arylene (e.g., C₆-C₁₀, C₁₀ aryl, or phenylene), or R²²-substituted or unsubstituted heteroarylene (e.g., 5 to 10 membered, 5 to 9 membered, or 5 to 6 membered heteroarylene). In embodiments, L² is a
bond, —NH—, —O—, —S—, —C(O)—, —C(O)NH—, —NHC(O)—, —NHC(O)NH—, —C(O)O—, —OC(O)—, R²²-substituted alkylene (e.g., C₁-C₈, C₁-C₆, or C₁-C₄ alkylene), R²²-substituted heteroalkylene (e.g., 2 to 8 membered, 2 to 6 membered, or 2 to 4 membered heteroalkylene), R²²-substituted cycloalkylene (e.g., C₃-C₈, C₃-C₆, or C₅-C₆ cycloalkylene), R²²-substituted heterocycloalkylene (e.g., 3 to 8 membered, 3 to 6 membered, or 5 to 6 membered heterocycloalkylene), R²²-substituted arylene (e.g., C₆-C₁₀, C₁₀ aryl, or phenylene), or R²²-substituted heteroarylene (e.g., 5 to 10 membered, 5 to 9 membered, or 5 to 6 membered heteroarylene). In embodiments, L² is a bond, —NH—, —O—, —S—, —C(O)—, —C(O)NH—, —NHC(O)—, —NHC(O)NH—, —C(O)O—, —OC(O)—, unsubstituted alkylene (e.g., C₁-C₈, C₁-C₆, or C₁-C₄ alkylene), unsubstituted heteroalkylene (e.g., 2 to 8 membered, 2 to 6 membered, or 2 to 4 membered heteroalkylene), unsubstituted cycloalkylene (e.g., C₃-C₈, C₃-C₆, or C₅-C₆ cycloalkylene), unsubstituted heterocycloalkylene (e.g., 3 to 8 membered, 3 to 6 membered, or 5 to 6 membered heterocycloalkylene), unsubstituted arylene (e.g., C₆-C₁₀, C₁₀ aryl, or phenylene), or unsubstituted heteroarylene (e.g., 5 to 10 membered, 5 to 9 membered, or 5 to 6 membered heteroarylene).

R²² is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R²³-substituted or unsubstituted alkyl (e.g., C₁-C₈, C₁-C₆, or C₁-C₄ alkyl), R²³-substituted or unsubstituted heteroalkyl (e.g., 2 to 8 membered, 2 to 6 membered, or 2 to 4 membered heteroalkyl), R²³-substituted or unsubstituted cycloalkyl (e.g., C₃-C₈, C₃-C₆, or C₅-C₆ cycloalkyl), R²³-substituted or unsubstituted heterocycloalkyl (e.g., 3 to 8 membered, 3 to 6 membered, or 5 to 6 membered heterocycloalkyl), R²³-substituted or unsubstituted aryl (e.g., C₆-C₁₀, C₁₀ aryl, or phenyl), or R²³-substituted or unsubstituted heteroaryl (e.g., 5 to 10 membered, 5 to 9 membered, or 5 to 6 membered heteroaryl). In embodiments, R²² is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R²³-substituted alkyl (e.g., C₁-C₈, C₁-C₆, or C₁-C₄ alkyl), R²³-substituted heteroalkyl (e.g., 2 to 8 membered, 2 to 6 membered, or 2 to 4 membered heteroalkyl), R²³-substituted cycloalkyl (e.g., C₃-C₈, C₃-C₆, or C₅-C₆ cycloalkyl), R²³-substituted heterocycloalkyl (e.g., 3 to 8 membered, 3 to 6 membered, or 5 to 6 membered heterocycloalkyl), R²³-substituted aryl (e.g., C₆-C₁₀, C₁₀ aryl, or phenyl), or R³-substituted heteroaryl (e.g., 5 to 10 membered, 5 to 9 membered, or 5 to 6 membered heteroaryl). In embodiments, R²² is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

Each R^{1A}, R^{1B}, R^{1C}, R^{1D}, R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently hydrogen, —CX₃, —CHX₂, —CH₂X (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I), —CN, —OH, —COOH, —CONH₂, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). X is independently —F, —Cl, —Br, or —I. In embodiments, each R^{1A}, R^{1B}, R^{1C}, R^{1D}, R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C} and R^{19D} are independently hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —CN, —OH, —COOH, —CONH₂, R²⁴-substituted or unsubstituted alkyl (e.g., C₁-C₂₀, C₁-C₁₂, C₁-C₈, C₁-C₆, C₁-C₄, or C₁-C₂), R²⁴-substituted or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), R²⁴-substituted or unsubstituted cycloalkyl (e.g., C₃-C₁₀, C₃-C₈, C₃-C₆, C₄-C₆, or C₅-C₆), R²⁴-substituted or unsubstituted heterocycloalkyl (e.g., 3 to 10 membered, 3 to 8 membered, 3 to 6 membered, 4 to 6 membered, 4 to 5 membered, or 5 to 6 membered), R²⁴-substituted or unsubstituted aryl (e.g., C₆-C₁₂, C₆-C₁₀, or phenyl), or R²⁴-substituted or unsubstituted heteroaryl (e.g., 5 to 12 membered, 5 to 10 membered, 5 to 9 membered, or 5 to 6 membered). In embodiments, each R^{1A}, R^{1B}, R^{1C}, R^{1D}, R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently hydrogen, —CF₃, —CHF₂, —CH₂F, —CCl₃, —CHCl₂, —CH₂C₁, —CBr₃, —CHBr₂, —CH₂Br, —CI₃, —CHI₂, —CH₂I, —CN, —OH, —COOH, —CONH₂, R²⁴-substituted alkyl (e.g., C₁-C₂₀, C₁-C₁₂, C₁-C₈, C₁-C₆, C₁-C₄, or C₁-C₂), R²⁴-substituted heteroalkyl (e.g., 2 to 20 membered, 2 to 12 membered, 2 to 8 membered, 2 to 6 membered, 4 to 6 membered, 2 to 3 membered, or 4 to 5 membered), R²⁴-substituted cycloalkyl (e.g., C₃-C₁₀, C₃-C₈, C₃-C₆, C₄-C₆, or C₅-C₆), R²⁴-substituted heterocycloalkyl (e.g., 3 to 10 membered, 3 to 8 membered, 3 to 6 membered, 4 to 6 membered, 4 to 5 membered, or 5 to 6 membered), R²⁴-substituted aryl (e.g., C₆-C₁₂, C₆-C₁₀, or phenyl), or R²⁴-substituted heteroaryl (e.g., 5 to 12 membered, 5 to 10 membered, 5 to 9 membered, or 5 to 6 membered). In embodiments, each R^{1A}, R^{1B}, R^{1C}, R^{1D}, R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —CN, —OH, —COOH, —CONH₂, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, each R^{1A}, R^{1B}, R^{1C}, R^{1D}, R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently hydrogen.

Each R^{1A} and R^{1B}, R^{16A} and R^{16B}, R^{17A} and R^{17B}, R^{18A} and R^{18B}, and R^{19A} and R^{19B} together with nitrogen attached thereto may be joined to form R²⁴-substituted or unsubstituted heterocycloalkyl (e.g., 3 to 10 membered, 3 to 8 membered, 3 to 6 membered, 4 to 6 membered, 4 to 5 membered, or 5 to 6 membered), or R²⁴-substituted or unsubstituted heteroaryl (e.g., 5 to 12 membered, 5 to 10 membered, 5 to 9 membered, or 5 to 6 membered). Each R^{1A} and R^{1B}, R^{16A} and R^{16B}, R^{17A} and R^{17B}, R^{18A} and R^{18B}, and R^{19A} and R^{19B} together with nitrogen attached thereto may be joined to form R²⁴-substituted heterocycloalkyl (e.g., 3 to 10 membered, 3 to 8 membered, 3 to 6 membered, 4 to 6 membered, 4 to 5 membered, or 5 to 6 membered), or R²⁴-substituted heteroaryl (e.g., 5 to 12 membered, 5 to 10 membered, 5 to 9 membered, or 5 to 6 membered). Each R^{1A} and R^{1B}, R^{16A} and R^{16B}, R^{17A} and R^{17B}, R^{18A} and R^{18B}, and R^{19A} and R^{19B} together with nitrogen attached thereto may be joined to form unsubstituted heterocycloalkyl (e.g., 3 to 10 membered, 3 to 8 membered, 3 to 6 membered, 4 to 6 membered, 4 to 5 membered, or 5 to 6 membered), or unsubstituted heteroaryl (e.g., 5 to 12 membered, 5 to 10 membered, 5 to 9 membered, or 5 to 6 membered). In embodiments, each R^{1A} and R^{1B}, R^{16A} and R^{16B}, R^{17A} and R^{17B}, R^{18A} and R^{18B}, and R^{19A} and R^{19B} together with nitrogen attached thereto may be joined to form R²⁴-substituted or unsubstituted pyridyl. In embodiments, each R^{1A} and R^{1B}, R^{16A} and R^{16B}, R^{17A} and R^{17B}, R^{18A} and R^{18B}, and R^{19A} and R^{19B} together with nitrogen attached thereto may be joined to form R²⁴-substituted or unsubstituted piperidinyl. In embodiments, each R^{1A} and R^{1B}, R^{16A} and R^{16B}, R^{17A} and R^{17B}, R^{18A} and R^{18B}, and R^{19A} and R^{19B} together with nitrogen attached thereto may be joined to form R²⁴-substituted or unsubstituted morpholinyl. In embodiments, each R^{1A} and R^{1B}, R^{16A} and R^{16B}, R^{17A} and R^{17B}, R^{18A} and R^{18B}, and R^{19A} and R^{19B} joined to form R²⁴-substituted or unsubstituted pyrrolyl. In embodiments, each R^{1A} and R^{1B}, R^{16A} and R^{16B}, R^{17A} and R^{17B}, R^{18A} and R^{18B}, and R^{19A} and R^{19B} together with nitrogen attached thereto may be joined to form R²⁴-substituted or unsubstituted pyrimidinyl.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{1A} and R^{1B} substituents bonded to the same nitrogen atom are joined is substituted, it is substituted with at least one substituent group. In embodiments, when the ring formed when R^{1A} and R^{1B} substituents bonded to the same nitrogen atom are joined is substituted, it is substituted with at least one size-limited substituent group. In embodiments, when the ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, a substituted R^{16A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16A} is substituted with a plurality of groups selected from substituent groups, size-limited substituent groups, and lower substituent groups; each substituent group, size-limited substituent group, and/or lower substituent group may optionally be different. In embodiments, when R^{16A} is substituted, it is substituted with at least one substituent group. In embodiments, when R^{16A} is substituted, it is substituted with at least one size-limited substituent group. In embodiments, when R^{16A} is substituted, it is substituted with at least one lower substituent group.

In embodiments, a substituted R^{16B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16B} is substituted with a plurality of groups selected from substituent groups, size-limited substituent groups, and lower substituent groups; each substituent group, size-limited substituent group, and/or lower substituent group may optionally be different. In embodiments, when R^{16B} is substituted, it is substituted with at least one substituent group. In embodiments, when R^{16B} is substituted, it is substituted with at least one size-limited substituent group. In embodiments, when R^{16B} is substituted, it is substituted with at least one lower substituent group.

In embodiments, a substituted ring formed when R^{16A} and R^{16B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{16A} and R^{16B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{16A} and R^{16B} substituents bonded to the same nitrogen atom are joined is substituted, it is substituted with at least one substituent group. In embodiments, when the ring formed when R^{16A} and R^{16B} substituents bonded to the same nitrogen atom are joined is substituted, it is substituted with at least one size-limited substituent group. In embodiments, when the ring formed when R^{16A} and R^{1B} substituents bonded to the same nitrogen atom are joined is substituted, it is substituted with at least one lower substituent group.

In embodiments, a substituted R^{16C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16C} is substituted with a plurality of groups selected from substituent groups, size-limited substituent groups, and lower substituent groups; each substituent group, size-limited substituent group, and/or lower substituent group may optionally be different. In embodiments, when R^{16C} is substituted, it is substituted with at least one substituent group. In embodiments, when R^{16C} is substituted, it is substituted with at least one size-limited substituent group. In embodiments, when R^{16C} is substituted, it is substituted with at least one lower substituent group.

In embodiments, a substituted R^{16D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16D} is substituted with a plurality of groups selected from substituent groups, size-limited substituent groups, and lower substituent groups; each substituent group, size-limited substituent group, and/or lower substituent group may optionally be different. In embodiments, when R^{16D} is substituted, it is substituted with at least one substituent group. In embodiments, when R^{16D} is substituted, it is substituted with at least one size-limited substituent group. In embodiments, when R^{16D} is substituted, it is substituted with at least one lower substituent group.

In embodiments, a substituted R^{17A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17A} is substituted with a plurality of groups selected from substituent groups, size-limited substituent groups, and lower substituent groups; each substituent group, size-limited substituent group, and/or lower substituent group may optionally be different. In embodiments, when R^{17A} is substituted, it is substituted with at least one substituent group. In embodiments, when R^{17A} is substituted, it is substituted with at least one size-limited substituent group. In embodiments, when R^{17A} is substituted, it is substituted with at least one lower substituent group.

In embodiments, a substituted R^{17B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17B} is substituted with a plurality of groups selected from substituent groups, size-limited substituent groups, and lower substituent groups; each substituent group, size-limited substituent group, and/or lower substituent group may optionally be different. In embodiments, when R^{17B} is substituted, it is substituted with at least one substituent group. In embodiments, when R^{17B} is substituted, it is substituted with at least one size-limited substituent group. In embodiments, when R^{17B} is substituted, it is substituted with at least one lower substituent group.

In embodiments, a substituted ring formed when R^{17A} and R^{17B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{17A} and R^{17B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{17A} and R^{17B} substituents bonded to the same nitrogen atom are joined is substituted, it is substituted with at least one substituent group. In embodiments, when the ring formed when R^{17A} and R^{17B} substituents bonded to the same nitrogen atom are joined is substituted, it is substituted with at least one size-limited substituent group. In embodiments, when the ring formed when R^{17A} and R^{17B} substituents bonded to the same nitrogen atom are joined is substituted, it is substituted with at least one lower substituent group.

In embodiments, a substituted R^{17C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17C} is substituted with a plurality of groups selected from substituent groups, size-limited substituent groups, and lower substituent groups; each substituent group, size-limited substituent group, and/or lower substituent group may optionally be different. In embodiments, when R^{17C} is substituted, it is substituted with at least one substituent group. In embodiments, when R^{17C} is substituted, it is substituted with at least one size-limited substituent group. In embodiments, when R^{17C} is substituted, it is substituted with at least one lower substituent group.

In embodiments, a substituted R^{17D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17D} is substituted with a plurality of groups selected from substituent groups, size-limited substituent groups, and lower substituent groups; each substituent group, size-limited substituent group, and/or lower substituent group may optionally be different. In embodiments, when R^{17D} is substituted, it is substituted with at least one substituent group. In embodiments, when R^{17D} is substituted, it is substituted with at least one size-limited substituent group. In embodiments, when R^{17D} is substituted, it is substituted with at least one lower substituent group.

In embodiments, a substituted R^{18A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18A} is substituted with a plurality of groups selected from substituent groups, size-limited substituent groups, and lower substituent groups; each substituent group, size-limited substituent group, and/or lower substituent group may optionally be different. In embodiments, when R^{18A} is substituted, it is substituted with at least one substituent group. In embodiments, when R^{18A} is substituted, it is substituted with at least one size-limited substituent group. In embodiments, when R^{18A} is substituted, it is substituted with at least one lower substituent group.

In embodiments, a substituted R^{18B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18B} is substituted with a plurality of groups selected from substituent groups, size-limited substituent groups, and lower substituent groups; each substituent group, size-limited substituent group, and/or lower substituent group may optionally be different. In embodiments, when R^{18B} is substituted, it is substituted with at least one substituent group. In embodiments, when R^{18B} is substituted, it is substituted with at least one size-limited substituent group. In embodiments, when R^{18B} is substituted, it is substituted with at least one lower substituent group.

In embodiments, a substituted ring formed when R^{18A} and R^{18B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{18A} and R^{18B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{18A} and R^{18B} substituents bonded to the same nitrogen atom are joined is substituted, it is substituted with at least one substituent group. In embodiments, when the ring formed when R^{18A} and R^{18B} substituents bonded to the same nitrogen atom are joined is substituted, it is substituted with at least one size-limited substituent group. In embodiments, when the ring formed when R^{18A} and R^{18B} substituents bonded to the same nitrogen atom are joined is substituted, it is substituted with at least one lower substituent group.

In embodiments, a substituted R^{18C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18C} is substituted with a plurality of groups selected from substituent groups, size-limited substituent groups, and lower substituent groups; each substituent group, size-limited substituent group, and/or lower substituent group may optionally be different. In embodiments, when R^{18C} is substituted, it is substituted with at least one substituent group. In embodiments, when R^{18C} is substituted, it is substituted with at least one size-limited substituent group. In embodiments, when R^{18C} is substituted, it is substituted with at least one lower substituent group.

In embodiments, a substituted R^{18D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18D} is substituted with a plurality of groups selected from substituent groups, size-limited substituent groups, and lower substituent groups; each substituent group, size-limited substituent group, and/or lower substituent group may optionally be different. In embodiments, when R^{18D} is substituted, it is substituted with at least one substituent group. In embodiments, when R^{18D} is substituted, it is substituted with at least one size-limited substituent group. In embodiments, when R^{18D} is substituted, it is substituted with at least one lower substituent group.

In embodiments, a substituted R^{19A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19A} is substituted with a plurality of groups selected from substituent groups, size-limited substituent groups, and lower substituent groups; each substituent group, size-limited substituent group, and/or lower substituent group may optionally be different. In embodiments, when R^{19A} is substituted, it is substituted with at least one substituent group. In embodiments, when R^{19A} is substituted, it is substituted with at least one size-limited substituent group. In embodiments, when R^{19A} is substituted, it is substituted with at least one lower substituent group.

In embodiments, a substituted R^{19B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19B} is substituted with a plurality of groups selected from substituent groups, size-limited substituent groups, and lower substituent groups; each substituent group, size-limited substituent group, and/or lower substituent group may optionally be different. In embodiments, when R^{19B} is substituted, it is substituted with at least one substituent group. In embodiments, when R^{19B} is substituted, it is substituted with at least one size-limited substituent group. In embodiments, when R^{19B} is substituted, it is substituted with at least one lower substituent group.

In embodiments, a substituted ring formed when R^{19A} and R^{19B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{19A} and R^{19B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{19A} and R^{19B} substituents bonded to the same nitrogen atom are joined is substituted, it is substituted with at least one substituent group. In embodiments, when the ring formed when R^{19A} and R^{19B} substituents bonded to the same nitrogen atom are joined is substituted, it is substituted with at least one size-limited substituent group. In embodiments, when the ring formed when R^{19A} and R^{19B} substituents bonded to the same nitrogen atom are joined is substituted, it is substituted with at least one lower substituent group.

In embodiments, a substituted R^{19C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19C} is substituted with a plurality of groups selected from substituent groups, size-limited substituent groups, and lower substituent groups; each substituent group, size-limited substituent group, and/or lower substituent group may optionally be different. In embodiments, when R^{19C} is substituted, it is substituted with at least one substituent group. In embodiments, when R^{19C} is substituted, it is substituted with at least one size-limited substituent group. In embodiments, when R^{19C} is substituted, it is substituted with at least one lower substituent group.

In embodiments, a substituted R^{19D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19D} is substituted with a plurality of groups selected from substituent groups, size-limited substituent groups, and lower substituent groups; each substituent group, size-limited substituent group, and/or lower substituent group may optionally be different. In embodiments, when R^{19D} is substituted, it is substituted with at least one substituent group. In embodiments, when R^{19D} is substituted, it is substituted with at least one size-limited substituent group. In embodiments, when R^{19D} is substituted, it is substituted with at least one lower substituent group.

R^{1F}, R^{16F}, R^{17F}, R^{18F}, R^{19F}, R²¹, R²³, and R²⁴ are independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀ aryl, C₁₀ aryl or phenyl), or unsubstituted heteroaryl (e.g., 5 to 10 membered, 5 to 9 membered, or 5 to 6 membered heteroaryl).

X, X¹, X¹⁶, X¹⁷, X¹⁸, are X¹⁹ are independently —F, —Cl, —Br, or —I. In embodiments, X is —F. In embodiments, X is —Cl. In embodiments, X is —Br. In embodiments, X is —I. In embodiments, X¹ is —F. In embodiments, X¹ is —Cl. In embodiments, X¹ is —Br. In embodiments, X¹ is —I. In embodiments, X¹⁶ is —F. In embodiments, X¹⁶ is —Cl. In embodiments, X¹⁶ is —Br. In embodiments, X¹⁶ is —I. In embodiments, X¹⁷ is —F. In embodiments, X¹⁷ is —Cl. In embodiments, X¹⁷ is —Br. In embodiments, X¹⁷ is —I. In embodiments, X¹⁸ is —F. In embodiments, X¹⁸ is —Cl. In embodiments, X¹⁸ is —Br. In embodiments, X¹⁸ is —I. In embodiments, X¹⁹ is —F. In embodiments, X¹⁹ is —Cl. In embodiments, X¹⁹ is —Br. In embodiments, X¹⁹ is —I.

n1, n16, n17, n18, and n19 are independently an integer from 0 to 4 (e.g. 0). In embodiments, n1 is 0. In embodiments, n1 is 1. In embodiments, n1 is 2. In embodiments, n1 is 3. In embodiments, n1 is 4. In embodiments, n16 is 0. In embodiments, n16 is 1. In embodiments, n16 is 2. In embodiments, n16 is 3. In embodiments, n16 is 4. In embodiments, n17 is 0. In embodiments, n17 is 1. In embodiments, n17 is 2. In embodiments, n17 is 3. In embodiments, n17 is 4. In embodiments, n18 is 0. In embodiments, n18 is 1. In embodiments, n18 is 2. In embodiments, n18 is 3. In embodiments, n18 is 4. In embodiments, n19 is 0. In embodiments, n19 is 1. In embodiments, n19 is 2. In embodiments, n19 is 3. In embodiments, n19 is 4.

m1, m16, m17, m18, and m19 are independently an integer from 1 to 2. In embodiments, m1 is 1. In embodiments, m1 is 2. In embodiments, m2 is 1. In embodiments, m16 is 1. In embodiments, m16 is 2. In embodiments, m17 is 1. In embodiments, m17 is 2. In embodiments, m18 is 1. In embodiments, m18 is 2. In embodiments, m19 is 1. In embodiments, m19 is 2.

v1, v16, v17, v18, and v19 are independently an integer from 1 to 2. In embodiments, v1 is 1. In embodiments, v1 is 2. In embodiments, v16 is 1. In embodiments, v16 is 2. In embodiments, v17 is 1. In embodiments, v17 is 2. In embodiments, v18 is 1. In embodiments, v18 is 2. In embodiments, v19 is 1. In embodiments, v19 is 2.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Proteins

A compound as described herein may form a covalent bond with an amino acid moiety of a Gαs protein (e.g., human Gαs). Thus, in an aspect is provided a Gαs protein covalently bonded to a compound as described herein. In embodiments, the Gαs is in the GTP state. In embodiments, the Gαs is in the GDP state.

In embodiments, the compound is bonded to a cysteine residue of the protein. In embodiments, the Gαs protein as being covalently bonded to the compound has the structure:

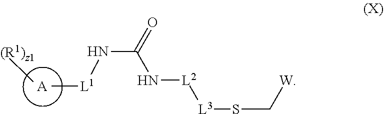

W together with the —CH₂S— to which it is attached form said Gαs protein covalently bonded to a compound; and L³ is substituted or unsubstituted alkylene or substituted or unsubstituted heteroalkylene. L¹, L², R¹, and z1 are as described above.

In embodiments, L³ is

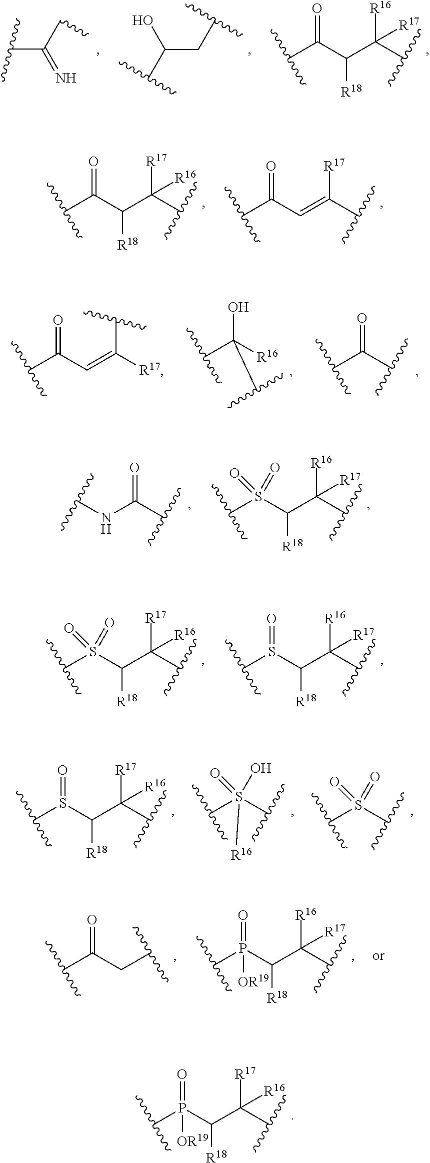

R¹⁶, R¹⁷, R¹⁸ and R¹⁹ are as described above.

In embodiments, L³ is

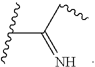

In embodiments, L³ is

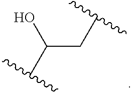 [SMILES: *CC(*)O]

In embodiments, L³ is

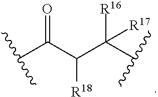

In embodiments, L³ is

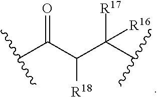

In embodiments, L³ is

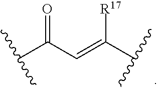

In embodiments, L³ is

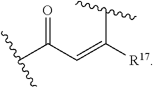

In embodiments, L³ is

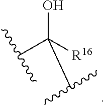

In embodiments, L³ is

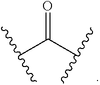 [SMILES: *C(*)=O]

In embodiments, L³ is

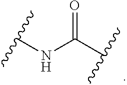 [SMILES: *NC(*)=O]

In embodiments, L³ is

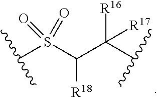

In embodiments, L³ is

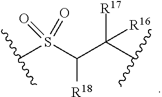

In embodiments, L³ is

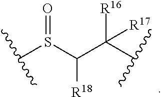

In embodiments, L³ is

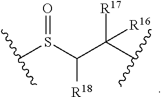

In embodiments, L³ is

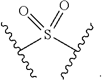 [SMILES: *S(*)(=O)=O]

In embodiments, L³ is

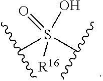

In embodiments, L³ is

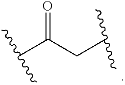 [SMILES: *CC(*)=O]

In embodiments, L³ is

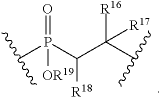

In embodiments, L³ is

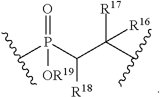

In embodiments, L³ is a substituted (e.g., substituted with a substituent group, a size-limited substituent group, or lower substituent group) or unsubstituted alkylene (e.g., C₁-C₈, C₁-C₆, or C₁-C₄ alkylene), substituted (e.g., substituted with a substituent group, a size-limited substituent group, or lower substituent group) or unsubstituted heteroalkylene (e.g., 2 to 8 membered, 2 to 6 membered, or 2 to 4 membered heteroalkylene). In embodiments, L³ is a substituted (e.g., substituted with a substituent group, a size-limited substituent group, or lower substituent group) or unsubstituted alkylene (e.g., C₁-C₈, C₁-C₆, or C₁-C₄ alkylene). In embodiments, L³ is a substituted (e.g., substituted with a substituent group, a size-limited substituent group, or lower substituent group) or unsubstituted heteroalkylene (e.g., 2 to 8 membered, 2 to 6 membered, or 2 to 4 membered heteroalkylene).

In embodiments, a substituted L³ is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a substituted (e.g., substituted with a substituent group, a size-limited substituent group, or lower substituent group) or unsubstituted C₁-C₈ alkylene. In embodiments, L³ is a substituted (e.g., substituted with a substituent group, a size-limited substituent group, or lower substituent group) C₁-C₈ alkylene. In embodiments, L³ is an unsubstituted C₁-C₈ alkylene. In embodiments, L³ is a substituted (e.g., substituted with a substituent group, a size-limited substituent group, or lower substituent group) or unsubstituted C₁-C₆ alkylene. In embodiments, L³ is a substituted (e.g., substituted with a substituent group, a size-limited substituent group, or lower substituent group) C₁-C₆ alkylene. In embodiments, L³ is an unsubstituted C₁-C₆ alkylene. In embodiments, L³ is a substituted (e.g., substituted with a substituent group, a size-limited substituent group, or lower substituent group) or unsubstituted C₁-C₄ alkylene. In embodiments, L³ is a substituted (e.g., substituted with a substituent group, a size-limited substituent group, or lower substituent group) C₁-C₄ alkylene. In embodiments, L³ is an unsubstituted C₁-C₄ alkylene.

In embodiments, L³ is a substituted (e.g., substituted with a substituent group, a size-limited substituent group, or lower substituent group) or unsubstituted 2 to 8 membered heteroalkylene. In embodiments, L³ is a substituted (e.g., substituted with a substituent group, a size-limited substituent group, or lower substituent group) 2 to 8 membered heteroalkylene. In embodiments, L³ is an unsubstituted 2 to 8 membered heteroalkylene. In embodiments, L³ is a substituted (e.g., substituted with a substituent group, a size-limited substituent group, or lower substituent group) or unsubstituted 2 to 6 membered heteroalkylene. In embodiments, L³ is a substituted (e.g., substituted with a substituent group, a size-limited substituent group, or lower substituent group) 2 to 6 membered heteroalkylene. In embodiments, L³ is an unsubstituted 2 to 6 membered heteroalkylene. In embodiments, L³ is a substituted (e.g., substituted with a substituent group, a size-limited substituent group, or lower substituent group) or unsubstituted 2 to 4 membered heteroalkylene. In embodiments, L³ is a substituted (e.g., substituted with a substituent group, a size-limited substituent group, or lower substituent group) 2 to 4 membered heteroalkylene. In embodiments, L³ is an unsubstituted 2 to 4 membered heteroalkylene.

In embodiments, L³ is a R²⁵-substituted or unsubstituted C₁-C₈ alkylene. In embodiments, L³ is a R²⁵-substituted C₁-C₈ alkylene. In embodiments, L³ is an unsubstituted C₁-C₈ alkylene. In embodiments, L³ is a R²⁵-substituted or unsubstituted C₁-C₆ alkylene. In embodiments, L³ is a R²⁵-substituted C₁-C₆ alkylene. In embodiments, L³ is an unsubstituted C₁-C₆ alkylene. In embodiments, L³ is a R²⁵-substituted or unsubstituted C₁-C₄ alkylene. In embodiments, L³ is a R²⁵-substituted C₁-C₄ alkylene. In embodiments, L³ is an unsubstituted C₁-C₄ alkylene.

In embodiments, L³ is a R²⁵-substituted or unsubstituted 2 to 8 membered heteroalkylene. In embodiments, L³ is a R²⁵-substituted 2 to 8 membered heteroalkylene. In embodiments, L³ is an unsubstituted 2 to 8 membered heteroalkylene. In embodiments, L³ is a R²⁵-substituted or unsubstituted 2 to 6 membered heteroalkylene. In embodiments, L³ is a R²⁵-substituted 2 to 6 membered heteroalkylene. In embodiments, L³ is an unsubstituted 2 to 6 membered heteroalkylene. In embodiments, L³ is a R²⁵-substituted or unsubstituted 2 to 4 membered heteroalkylene. In embodiments, L³ is a R²⁵-substituted 2 to 4 membered heteroalkylene. In embodiments, L³ is an unsubstituted 2 to 4 membered heteroalkylene.

R²⁵ is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R²⁶-substituted or unsubstituted alkyl (e.g., C₁-C₈, C₁-C₆, or C₁-C₄ alkyl), R²⁶-substituted or unsubstituted heteroalkyl (e.g., 2 to 8 membered, 2 to 6 membered, or 2 to 4 membered heteroalkyl), R²⁶-substituted or unsubstituted cycloalkyl (e.g., C₃-C₈, C₃-C₆, or C₅-C₆ cycloalkyl), R²⁶-substituted or unsubstituted heterocycloalkyl (e.g., 3 to 8 membered, 3 to 6 membered, or 5 to 6 membered heterocycloalkyl), R²⁶-substituted or unsubstituted aryl (e.g., C₆-C₁₀, C₁₀ aryl, or phenyl), or R²⁶-substituted or unsubstituted heteroaryl (e.g., 5 to 10 membered, 5 to 9 membered, or 5 to 6 membered heteroaryl). In embodiments, R²⁵ is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R²⁶-substituted alkyl (e.g., C₁-C₈, C₁-C₆, or C₁-C₄ alkyl), R²⁶-substituted heteroalkyl (e.g., 2 to 8 membered, 2 to 6 membered, or 2 to 4 membered heteroalkyl), R²⁶-substituted cycloalkyl (e.g., C₃-C₈, C₃-C₆, or C₅-C₆ cycloalkyl), R²⁶-substituted heterocycloalkyl (e.g., 3 to 8 membered, 3 to 6 membered, or 5 to 6 membered heterocycloalkyl), R²⁶-substituted aryl (e.g., C₆-C₁₀, C₁₀ aryl, or phenyl), or R⁶-substituted heteroaryl (e.g., 5 to 10 membered, 5 to 9 membered, or 5 to 6 membered heteroaryl). In embodiments, R²⁵ is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀, C₁₀ aryl, or phenyl), or unsubstituted heteroaryl (e.g., 5 to 10 membered, 5 to 9 membered, or 5 to 6 membered heteroaryl).

R²⁶ is independently oxo, halogen, —CF₃, —CCl₃, —CBr₃, —C₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, or C₁-C₄ alkyl), unsubstituted heteroalkyl (e.g., 2 to 8 membered, 2 to 6 membered, or 2 to 4 membered heteroalkyl), unsubstituted cycloalkyl (e.g., C₃-C₈, C₃-C₆, or C₅-C₆ cycloalkyl), unsubstituted heterocycloalkyl (e.g., 3 to 8 membered, 3 to 6 membered, or 5 to 6 membered heterocycloalkyl), unsubstituted aryl (e.g., C₆-C₁₀ aryl, C₁₀ aryl or phenyl), or unsubstituted heteroaryl (e.g., 5 to 10 membered, 5 to 9 membered, or 5 to 6 membered heteroaryl).

In embodiments, the Gαs protein has the amino acid sequence of SEQ ID NO:1 including the sequence below with one or more mutations (e.g., R201C and C237S). The amino acids, R201 and C237, in the SEQ ID NO: 1 may be where the mutations (e.g., R201C and C237S) can occur.

In embodiments, the Gαs protein includes R201C mutation in SEQ ID NO: 1. In embodiments, the Gαs protein includes Cys201. In embodiments, the Gαs protein does not include Cys237.

In embodiments, the compound is bonded to Cys201 of the mutant human Gαs (e.g., R201C mutation in SEQ ID NO: 1) or a selected residue in a selected protein corresponding to Cys201. In embodiments, the compound is bonded to cysteine 201 of the mutant human Gαs (e.g., R201C mutation in SEQ ID NO: 1). In embodiments, the compound is bonded to an amin acid residue corresponding to Cys201 in the selected Gαs.

In embodiments, the compound is bonded to Cys237 of the mutant human Gαs (e.g., C237 in SEQ ID NO: 1) or a selected residue in a selected protein corresponding to Cys237. In embodiments, the compound is bonded to cysteine 237 of the human Gαs (e.g., C237 in SEQ ID NO: 1). In embodiments, the compound is bonded to an amin acid residue corresponding to cystein237 in the selected Gαs.

In embodiments, the Gαs protein has the amino acid sequence of SEQ ID NO:2 including the sequence below with one or more mutations (e.g., R187C and C223S). The amino acids, R187 and C223, in the SEQ ID NO: 1 may be where the mutations (e.g., R187C and C223S) can occur.

In embodiments, the Gαs protein includes R187C mutation in SEQ ID NO: 2. In embodiments, the Gαs protein includes Cys187. In embodiments, the Gαs protein does not include Cys223.

In embodiments, the compound is bonded to Cys187 of the mutant human Gαs (e.g., R187C mutation in SEQ ID NO: 2) or a selected residue in a selected protein corresponding to Cys187. In embodiments, the compound is bonded to Cys187 of the mutant human Gαs (e.g., R187C mutation in SEQ ID NO: 2). In embodiments, the compound is bonded to an amin acid residue corresponding to Cys187 in the selected Gαs.

In embodiments, the compound is bonded to Cys223 of the human Gαs (e.g., C223 in SEQ ID NO: 2) or a selected residue in a selected protein corresponding to Cys223. In embodiments, the compound is bonded to Cys223 of the human Gαs (e.g., C223 in SEQ ID NO: 2). In embodiments, the compound is bonded to an amin acid residue corresponding to Cys223 in the selected Gαs.

In an aspect is provided a Gαs protein that is covalently bonded to a portion of a compound as described herein.

In embodiments, the Gαs protein is covalently bonded to a Gαs small molecule inhibitor **not defined** at R201C. In embodiments, the Gαs protein is a GTP-bound Gαs protein. In embodiments, the Gαs protein is a GDP-bound Gαs protein.

In embodiments, the Gαs protein is covalently bonded to a human Gαs small molecule inhibitor at R201C. In embodiments, the Gαs protein is a GTP-bound human Gαs protein. In embodiments, the Gαs protein is a GDP-bound human Gαs protein.

In embodiments, the Gαs protein is covalently bonded to a human Gαs small molecule inhibitor at a corresponding residue of R201C in SEQ ID NO: 1. In embodiments, the Gαs protein is a GTP-bound human Gαs protein. In embodiments, the Gαs protein is a GDP-bound human Gαs protein.

In embodiments, the Gαs protein covalently bonded to a Gαs small molecule inhibitor at C237. In embodiments, the Gαs protein is a GTP-bound Gαs protein. In embodiments, the Gαs protein is a GDP-bound Gαs protein.

In embodiments, the Gαs protein covalently bonded to a human Gαs small molecule inhibitor at C237. In embodiments, the Gαs protein is a GTP-bound human Gαs protein. In embodiments, the Gαs protein is a GDP-bound human Gαs protein.

In embodiments, the Gαs protein covalently bonded to a human Gαs small molecule inhibitor at a corresponding residue of C237 in SEQ ID NO: 1. In embodiments, the Gαs protein is a GTP-bound human Gαs protein. In embodiments, the Gαs protein is a GDP-bound human Gαs protein.

In embodiments, the Gαs protein is covalently bonded to a human Gαs small molecule inhibitor at a corresponding residue of R187C in SEQ ID NO: 2. In embodiments, the Gαs protein is a GTP-bound human Gαs protein. In embodiments, the Gαs protein is a GDP-bound human Gαs protein.

In embodiments, the Gαs protein covalently bonded to a human Gαs small molecule inhibitor at a corresponding residue of C223 in SEQ ID NO: 2. In embodiments, the Gαs protein is a GTP-bound human Gαs protein. In embodiments, the Gαs protein is a GDP-bound human Gαs protein.

### IV. Pharmaceutical Compositions

In embodiments, the compound described herein (e.g., Formula (I), (II), (III-a), (III-b), (III-c), (IV), (V-a), (V-b), (VI), (VII), or (VIII)) is administered as a pure chemical. In embodiments, the compound as described herein (e.g., Formula (I), (II), (III-a), (III-b), (III-c), (IV), (V-a), (V-b), (VI), (VII), or (VIII)) is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Gennaro, A. R., “Remington: The Science and Practice of Pharmacy,” 21^{st} ed., Easton: Lippincott Williams & Wilkins, 2005.

In certain embodiments, the compound of Formula (I), (II), (III-a), (III-b), (III-c), (IV), (V-a), (V-b), (VI), (VII), or (VIII) as described herein is administered as a pure chemical. In some embodiments, the compound of Formula (I), (II), (III-a), (III-b), (III-c), (IV), (V-a), (V-b), (VI), (VII), or (VIII) described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Gennaro, A. R., “Remington: The Science and Practice of Pharmacy,” 21^{st} ed., Easton: Lippincott Williams & Wilkins, 2005.

Accordingly, provided herein is a pharmaceutical composition including at least one compound of Formula (I), (II), (III-a), (III-b), (III-c), (IV), (V-a), (V-b), (VI), (VII), or (VIII) described herein, or a pharmaceutically acceptable salt or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

Accordingly, provided herein is a pharmaceutical composition including at least one compound of Formula (I), (I), (III-a), (III-b), (III-c), (IV), (V-a), (V-b), (VI), (VII), or (VIII) described herein, or a pharmaceutically acceptable salt or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

In embodiments, the compound described herein (e.g., of Formula (I), (II), (III-a), (III-b), (III-c), (IV), (V-a), (V-b), (VI), (VII), or (VIII)) is substantially pure, in that it contains less than about 5%, or less than about 1%, or less than about 0.1%, of other organic small molecules, such as contaminating intermediates or by-products that are created, for example, in one or more of the steps of a synthesis method.

In certain embodiments, the compound of Formula (I), (II), (III-a), (III-b), (III-c), (IV), (V-a), (V-b), (VI), (VII), or (VIII) as described herein is substantially pure, in that it contains less than about 5%, or less than about 1%, or less than about 0.1%, of other organic small molecules, such as contaminating intermediates or by-products that are created, for example, in one or more of the steps of a synthesis method.

These pharmaceutical compositions include those suitable for oral, rectal, topical, buccal, parenteral (e.g., subcutaneous, intramuscular, intradermal, or intravenous), vaginal, ophthalmic, or aerosol administration.

Exemplary pharmaceutical compositions are used in the form of a pharmaceutical preparation, for example, in solid, semisolid or liquid form, which includes one or more of a disclosed compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active compound is included in the pharmaceutical composition in an amount sufficient to produce the desired effect upon the process or condition of the disease.

The dose of the composition including at least one compound described herein (e.g., of Formula (I), (II), (III-a), (III-b), (III-c), (IV), (V-a), (V-b), (VI), (VII), or (VIII)) differs, depending upon the patient's (e.g., human) condition, that is, stage of the disease, general health status, age, and other factors.

The dose of the composition including at least one compound of Formula (I), (II), (III-a), (III-b), (III-c), (IV), (V-a), (V-b), (VI), (VII), or (VIII) as described herein differs, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

Disclosed compounds are administered to subjects or patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors, with the appropriate dosage ultimately being at the discretion of the attendant physician. For treating clinical conditions and diseases noted above, a contemplated compound disclosed herein is administered orally, subcutaneously, topically, parenterally, by inhalation spray or rectally in dosage unit formulations containing conventional non-toxic pharmaceutically acceptable carriers, adjuvants and vehicles. Parenteral administration include subcutaneous injections, intravenous or intramuscular injections or infusion techniques.

### Effective Dosages

The pharmaceutical composition may include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### V. Methods of Use

In an aspect is provided a method of treating cancer. The method includes administering to a subject in need thereof an effective amount of a compound as described herein.

In embodiments, the subject is a human. In embodiments, the cancer is selected from human cancers and carcinomas, sarcomas, adenocarcinomas, lymphomas, leukemias, and the like. In embodiments, the cancer is a solid cancer or tumor. In embodiments, the cancer is pancreatic cancer. In embodiments, the cancer is a pituitary tumor. In embodiments, the cancer is a bone tumor.

In embodiments, the cancer or cancer cell is sensitive to Gαs inhibition.

In an aspect is provided a method of treating a bone condition. The method includes administering to a subject in need thereof an effective amount of a compound as described herein.

In embodiments, the bone condition is fibrous dysplasia. In embodiments, the fibrous dysplasia is monostotic fibrous dysplasia or polystotic fibrous dysplasia. In embodiments, the fibrous dysplasia is monostotic fibrous dysplasia. In embodiments, the fibrous dysplasia is polystotic fibrous dysplasia.

In an aspect is provided a method of treating McCune-Albright Syndrome. The method includes administering to a subject in need thereof an effective amount of a compound as described herein.

In an aspect is provided a method of treating cancer. The method include administering a Gαs cysteine 201 covalent inhibitor. In embodiments, the Gαs cysteine 201 covalent inhibitor is a compound as described herein.

In an aspect is provided a method of treating cancer. The method include administering a Gαs cysteine 237 covalent inhibitor. In embodiments, the Gαs cysteine 237 covalent inhibitor is a compound as described herein.

### VI. Embodiments P

Embodiment P1. A compound having the formula:

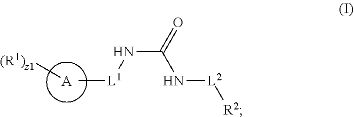

wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C (O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 6;
Ring A is aryl or heteroaryl;
L¹ is a
   bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a
   bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R² is an electrophilic moiety;
R^{1A}, R^{1B}, R^{1C}, and R^{1D} are independently
   hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X and X¹ are independently —F, —Cl, —Br, or —I;
n1 is independently an integer from 0 to 4; and
m1 and v1 are independently 1 or 2.

Embodiment P2. The compound of Embodiment 1, wherein z1 is an integer from 1 to 3.

Embodiment P3. The compound of Embodiment 1, wherein z1 is 0.

Embodiment P4. The compound of one of Embodiments 1 to 3, wherein Ring A is phenyl or 5 to 6-membered heteroaryl.

Embodiments P5. The compound of one of Embodiments 1 to 4, having the formula:

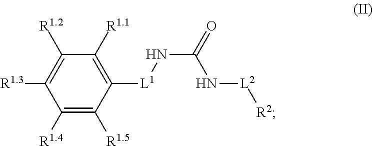

Each R^{1.1}, R^{1.2}, R^{1.3}, R^{1.4}, and R^{1.5} is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B},
   —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C (O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently
   hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X and X¹ are independently —F, —Cl, —Br, or —I;
n1 is independently an integer from 0 to 4; and
m1 and v1 are independently 1 or 2.

Embodiment P6. The compound of one of Embodiments 1 to 5, wherein L¹ is a bond, —NH—, —O—, —S—, —C(O)—, —C(O)NH—, —NHC(O)—, —NHC(O)NH—, —C(O)O—, —OC(O)—, substituted or unsubstituted alkylene, or substituted or unsubstituted heteroalkylene.

Embodiment P7. The compound of one of Embodiments 1 to 5, wherein L¹ is a bond, —NH—, —O—, —S—, —C(O)—, —C(O)NH—, —NHC(O)—, —NHC(O)NH—, —C(O)O—, —OC(O)—, substituted or unsubstituted C₁-C₆ alkylene, or substituted or unsubstituted 2 to 6 membered heteroalkylene.

Embodiment P8. The compound of one of Embodiments 1 to 5, wherein L¹ is a bond.

Embodiment P9. The compound of one of Embodiments 1 to 8, wherein L² is a bond, —NH—, —O—, —S—, —C(O)—, —C(O)NH—, —NHC(O)—, —NHC(O)NH—, —C(O)O—, —OC(O)—, substituted or unsubstituted alkylene, or substituted or unsubstituted heteroalkylene.

Embodiment P10. The compound of one of Embodiments 1 to 8, wherein L² is a bond, —NH—, —O—, —S—, —C(O)—, —C(O)NH—, —NHC(O)—, —NHC(O)NH—, —C(O)O—, —OC(O)—, substituted or unsubstituted C₁-C₆ alkylene, or substituted or unsubstituted 2 to 6 membered heteroalkylene.

Embodiment P11. The compound of one of Embodiments 1 to 8, wherein L² is an unsubstituted C₁-C₆ alkylene.

Embodiment P12. The compound of one of Embodiments 1 to 8, wherein L² is a bond.

Embodiment P13. The compound of one of Embodiments 1 to 12, wherein R² is

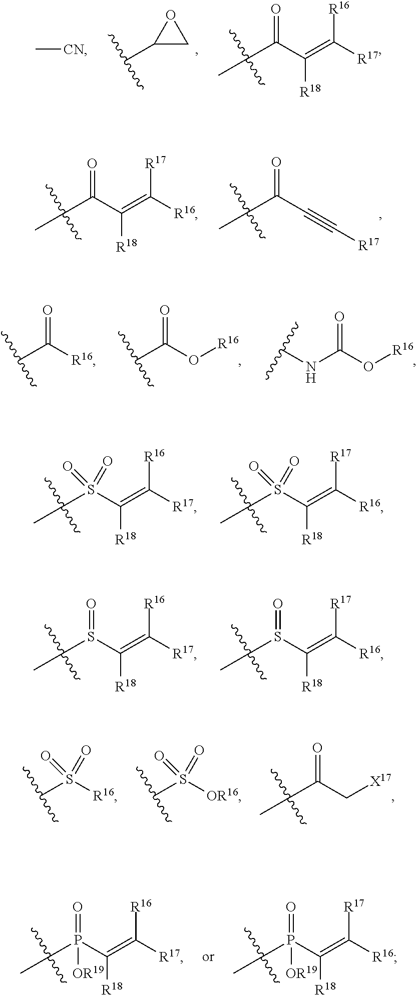

R¹⁶ is hydrogen,
   halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16B}, —NR^{16A}C(O)R^{16C}, —NR^{16A}C(O)OR^{16C}, —NR^{16A}OR^{16D}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is hydrogen,
   halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17B}, —NR^{17A}C(O)R^{17C}, —NR^{17A}C(O)OR^{17C}, —NR^{17A}OR^{17D}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is hydrogen,
   halogen, —CX¹⁸₃, —CHX¹⁸₂, —CH₂X¹⁸, —CN, —SOₙ₁₈R^{18D}, —SOᵥ₁₈NR^{18A}R^{18B}, —NHNR^{18A}R^{18B}, —ONR^{18A}R^{18B}, —NHC(O)NHNR^{18A}R^{18B},
   —NHC(O)NR^{18A}R^{18B}, —N(O)ₘ₁₈, —NR^{1A}R^{18B}, —C(O)R^{18C}, —C(O)—OR^{18C}, —C(O)NR^{18A}R^{18B}, —OR^{18D}, —NR^{18A}SO₂R^{18B}, —NR^{18A}C(O)R^{18C}, —NR^{1A}C(O)OR^{1C}, —NR^{18A}OR^{18D}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁹ is hydrogen,
   halogen, —CX¹⁹₃, —CHX¹⁹₂, —CH₂X¹⁹, —CN, —SOₙ₁₉R^{19D}, —SOᵥ₁₉NR^{19A}R^{19B}, —NHNR^{19A}R^{19B}, —ONR^{19A}R^{19B}, —NHC(O)NHNR^{19A}R^{19B}, —NHC(O)NR^{19A}R^{19B}, —N(O)ₘ₁₉, —NR^{19A}R^{19B}, —C(O)R^{19C}, —C(O)—OR^{19C}, —C(O)NR^{19A}R^{19B}, —OR^{19D}, —NR^{19A}SO₂R^{19B}, —NR^{19A}C(O)R^{19C}, —NR^{19A}C(O)OR^{19C}, —NR^{19A}OR^{19D}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
X¹⁶, X¹⁷, X¹⁸, and X¹⁹ are independently —F, —Cl, —Br, or —I;
n16, n17, n18, and n19 are independently an integer from 0 to 4; and
m16, m17, m18, m19, v16, v17, v18, and v19 are independently 1 or 2.

Embodiment P14. The compound of one of Embodiments 1 to 12, wherein R² is

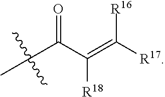

Embodiment P15. The compound of one of Embodiments 1 to 14, wherein:
R¹⁶ is hydrogen, unsubstituted C₁-C₄ alkyl, or unsubstituted C₃-C₆ cycloalkyl;
R¹⁷ is hydrogen, unsubstituted C₁-C₄ alkyl, or unsubstituted C₃-C₆ cycloalkyl; and
R¹⁸ is hydrogen, unsubstituted C₁-C₄ alkyl, or unsubstituted C₃-C₆ cycloalkyl.

Embodiment P16. The compound of one of Embodiments 1 to 14, wherein:
R¹⁶ is hydrogen or unsubstituted C₁-C₄ alkyl;
R¹⁷ is hydrogen or unsubstituted C₁-C₄ alkyl; and
R¹⁸ is hydrogen or unsubstituted C₁-C₄ alky.

Embodiment P17. The compound of one of Embodiments 1 to 14, wherein R¹⁶, R¹⁷, and R¹⁸ are hydrogen.

Embodiment P18. A pharmaceutical composition comprising the compound of any one of Embodiments 1 to 17 and a pharmaceutically acceptable excipient.

Embodiment P19. A method of inhibiting Gαs protein activity, said method comprising: contacting the Gαs protein with a compound of one of Embodiments 1 to 17.

Embodiment P20. A method of treating cancer, said method comprising administering to a subject in need thereof an effective amount of a compound of one of Embodiments 1 to 17.

Embodiment P21. The method of Embodiment 20, wherein the cancer is pancreatic cancer, a pituitary tumor, or a bone tumor.

Embodiment P22. The method of Embodiment 20, wherein the cancer is sensitive to Gαs inhibition.

Embodiment P23. A method of treating a bone condition, said method comprising administering to a subject in need thereof an effective amount of a compound of one of Embodiments 1 to 17.

Embodiment P24. The method of Embodiment 23, wherein the bone condition is fibrous dysplasia.

Embodiment P25. The method of Embodiment 24, wherein the fibrous dysplasia is monostotic fibrous dysplasia or polystotic fibrous dysplasia.

Embodiment P26. A method of treating McCune-Albright Syndrome, said method comprising administering to a subject in need thereof an effective amount of a compound of one of Embodiments 1 to 17.

Embodiment P27. A Gαs protein covalently bonded to a compound of one of Embodiments 1 to 17.

Embodiment P28. The Gαs protein of Embodiment 27, wherein Gαs is in the GTP state.

Embodiment P29. The Gαs protein of Embodiment 27, wherein Gαs is in the GDP state.

Embodiment P30. The Gαs protein of Embodiment 27, wherein the compound is bonded to a cysteine residue of the protein.

Embodiment P31. The Gαs protein of Embodiment 27, having the structure:

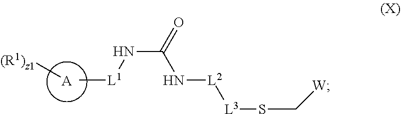

wherein,
W together with the —CH₂S— to which it is attached form said Gαs protein covalently bonded to a compound; and
L³ is substituted or unsubstituted alkylene or substituted or unsubstituted heteroalkylene.

Embodiment P32. The Gαs protein of Embodiment 31 wherein L³ is

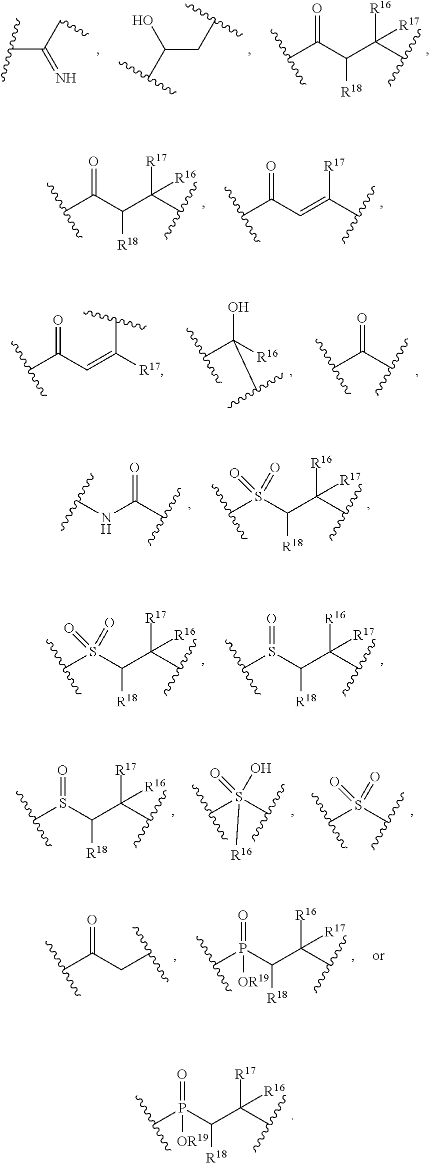

Embodiment P33. The Gαs protein of Embodiment 30, wherein the compound is bonded to cysteine 201.

Embodiment P34. The Gαs protein of Embodiment 30, wherein the compound is bonded to cysteine 237.

Embodiment P35. A Gαs protein covalently bonded to a portion of a compound of one of Embodiments 1 to 17.

Embodiment P36. A Gαs protein covalently bonded to a Gαs small molecule inhibitor at R201C.

Embodiment P37. The Gαs protein of Embodiment 36, wherein the Gαs protein is a GTP-bound Gαs protein.

Embodiment P38. The Gαs protein of Embodiment 36, wherein the Gαs protein is a GDP-bound Gαs protein.

Embodiment P39. A Gαs protein covalently bonded to a Gαs small molecule inhibitor at C237.

Embodiment P40. The Gαs protein of Embodiment 39, wherein the Gαs protein is a GTP-bound Gαs protein.

Embodiment P41. The Gαs protein of Embodiment 39, wherein the Gαs protein is a GDP-bound Gαs protein.

Embodiment P42. A method of treating cancer comprising administering a Gαs cysteine 201 covalent inhibitor.

Embodiment P43. The method of Embodiment 42, wherein the Gαs cysteine 201 covalent inhibitor is a compound of one of Embodiments 1 to 17.

Embodiment P44. A method of treating cancer comprising administering a Gαs cysteine 237 covalent inhibitor.

Embodiment P45. The method of Embodiment 44, wherein the Gαs cysteine 237 covalent inhibitor is a compound of one of Embodiments 1 to 17.

### EXAMPLES

State-selective Gαs labeling molecules based on disulfide tethering are demonstrated. The leading compounds can label the somatic cysteine mutant selectively over all other cysteines present in the protein. Based on (i) the structure activity relationships and (ii) GDP or GTP state dependent labelling, the leading compounds are excellent starting points for discovery of covalent irreversible (likely acrylamide) based drug candidates, which can yield enhanced drug candidate for Gαs associated cancer or disease. In particular, the leading compounds may be used to treat a cancer caused by mutations (e.g., R201C) in the GNAS gene.

### Example 1. Design of Inhibitors of Cancer-Associated Mutant GNAS

Deep sequencing reveals that G-protein mutations occur in several kinds of cancers^{[1]}. Some of the remarkable mutations were observed in GNAS, which encodes the α-subunit of the stimulatory G protein (Gαs). GNAS was first proved to be a putative oncogene that was abnormally activated in human growth hormone (GH)-secreting pituitary tumors in 1987^{[2, 3]}; activating mutations were identified in about 43% of 42 GH-secreting pituitary tumors, and they were responsible for the high secretory activity of such tumors^{[4]}. Since then, activating mutations of GNAS have been revealed to contribute to progression and metastasis of several other kinds of cancers. For example, to understand the pathogenesis of intraductal papillary mucinous neoplasm (IPMN, a precursor to invasive adenocarcinoma), Jian Wu et al. searched the mutations in IPMN patients, and found that 66% of 132 patients carried activating mutations at codon 201 of GNAS^{[5]}. According to the catalogue of somatic mutations in cancer (COSMIC) v62, approximately 4.2% of all cancer types harbor activating mutations in GNAS^{[1]}. About 64% of such GNAS mutations result in R201C^{[1]}. Arg201 can stabilize the pentavalent phosphate intermediate thus facilitates GTP hydrolysis; therefore mutation of this residue disrupts the GTPase activity of Gαs, keeping Gαs in a constitutively active state^{[6, 7]}. Using small molecules to specifically inhibit the cancer-associated mutant Gαs(R201C), would be a promising strategy for the therapy of cancers in which GNAS mutations occur. However, only a few small molecules have been reported to be Gαs inhibitors, and their selectivity between different subtypes of G-proteins is poor. No inhibitor that selectively targets Gαs(R201C) has been reported. In this proposal, I will take advantage of the similarity of Gαs(R201C) with K-Ras(G12C) and Gαq to design Gαs(R201C)-specific inhibitors. Such a design is based on the structure of Gαq with a non-covalent inhibitor^{[8]}, and the crystal structures of K-Ras(G12C) with inhibitors that's covalently linked with Cys12^{[9-11]}. Activity assays are also proposed to evaluate the potency of the designed inhibitors.

The G-proteins are composed of Gα, Gβ and Gγ subunits. Among them, Gα can form a heterotrimer with Gβγ when one molecule of GDP is located in the nucleotide-binding pocket of Gα. The GDP-bound Gα is inactive, and can be activated by the corresponding GPCR. As for Gαs, it can be activated by β2 adrenergic receptor (β2AR). Association of an agonist to the extracellular side of β2AR induces a conformational change of the intracellular side of the β2AR; then β2AR recruits the Gαs-Gβγ heterotrimer, and induces a rearrangement of the “P-loop” of the nucleotide-binding pocket of Gαs, leading to GDP release and GTP binding^{[12]}. The nucleotide exchange results in the dissociation of Gαs from both β2AR and the Gμγ dimer^{[6]}. After dissociation, the GTP-bound Gαs binds with and activates adenylyl cyclase (AC), which can catalyze the conversion of ATP to cAMP_{[13]}. Gαs has relatively slow GTPase activity. The hydrolysis of the GTP molecule in the nucleotide-binding pocket of Gαs to GDP induces the release of AC from Gαs. Then the GDP-bound Gαs associates with Gβγ, waiting for β2AR to activate it again.

The above cycle maintains the activity of Gαs at a proper level. But the cancer-associated mutation R201C breaks this cycle by locking Gαs in its GTP-bound state. The constitutive activation of Gαs can promote hyperplasia of cells in several kinds of cancers^{[1]}. So using small molecules to inhibit the abnormally activated Gαs would be a promising strategy for the therapy of cancer with a R201C mutation in GNAS. Because of the mosaic nature of GNAS in cancer patients^{[14]}, the inhibitors should specifically target the mutant Gαs but not the wild-type Gαs to avoid side effects.

So far, only a few types of G-protein inhibitors have been reported. They targeted different steps of G-protein activation. Some of them could disrupt the interaction between G-proteins and their receptors. For example, A small peptide, pGlu-Gln-D-Trp-Phe-D-Trp-D-Trp-Met-NH₂, was reported to competitively inhibit the binding of Gi (or Go) to M2 muscarinic cholinergic receptor or the binding of Gs to β2AR^{[15]}. Another compound, BIM-46187, was first reported to bind to the Gα subunit and block the receptor-G protein interaction with a poor selectivity^{[16]}, but in a recent study, BIM-46187 preferentially inhibited Gαq by blocking GTP entry^{[17]}. Some other compounds were used to inhibit replacement of GDP with GTP, such as compound YM-254890, which was reported to selectively block GDP-GTP exchange of Gαq/₁₁^{[18]}. Suramin and its analogues are another class of G-protein inhibitors, but the molecular mechanism of inhibition is controversial^{[19-21]}. None of these inhibitors was reported to selectively target Gαs, let alone specific inhibitors of the cancer-associated mutant Gαs(R201C).

It is aimed to develop small molecule inhibitors to specifically inhibit the cancer-associated mutant Gαs(R201C). To this end, the specific aims include 1) design of leading compounds that bind Gαs; 2) modification of the compounds to covalently bond Cys201 of Gαs(R201C); 3) structural analysis of the binding of these compounds with Gαs(R201C); 4) evaluation of the potency of these compounds in cellular models.

### Methods

Within G-protein family, Gαq shares a sequence identity of 42.5% with Gαs. Crystal structure of Gαq in complex with a specific inhibitor named YM254890 has been reported, giving us the only example of small molecule inhibitor-G-protein binding^{[8]}. In addition, G-proteins show similar features to other GTPase, such as the Ras family proteins. Their GDP-bound state is inactive, while the GTP-bound state is active in signal transduction. Structural analysis of Ras proteins revealed that two regions largely switched during the replacement of GDP with GTP^{[22]}. The two regions, named switch I and II, are also involved in nucleotide-exchange in G-proteins^{[7, 13]}.

Design of Leading Compounds that can Bind Cys201 of Gαs(R201C)

A crystal structure of Gαq-YM254890 complexed with Gαq has been reported because Gαq is closely related to Gαs. YM254890, a cyclic depsipeptide produced by Chromobacterium sp. QS3666, was first identified as a platelet aggregation inhibitor^{[23]}, then it turned out to be a novel Gαq/11-selective inhibitor^{[18]}. The cyclic scaffold of YM254890 is linked by ester bonds and amide bonds, and the nitrogen atoms of the amides are highly methylated. The moiety around the cyclic scaffold consists of aliphatic and aromatic residues, indicating that hydrophobic interactions involve in YM254890-Gαq binding^{[24]}.

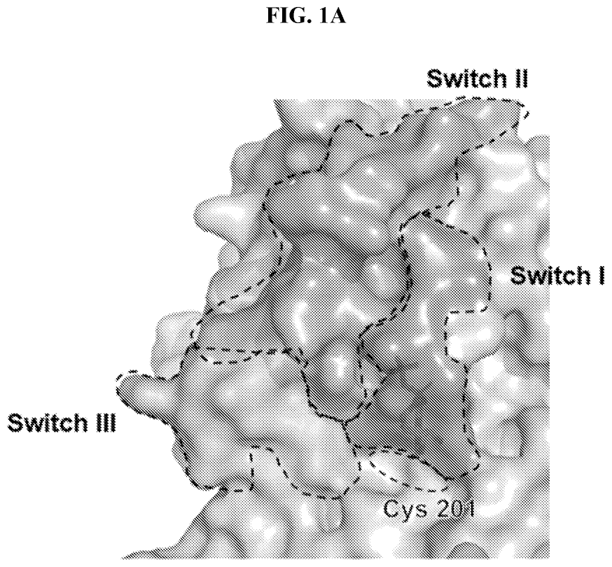

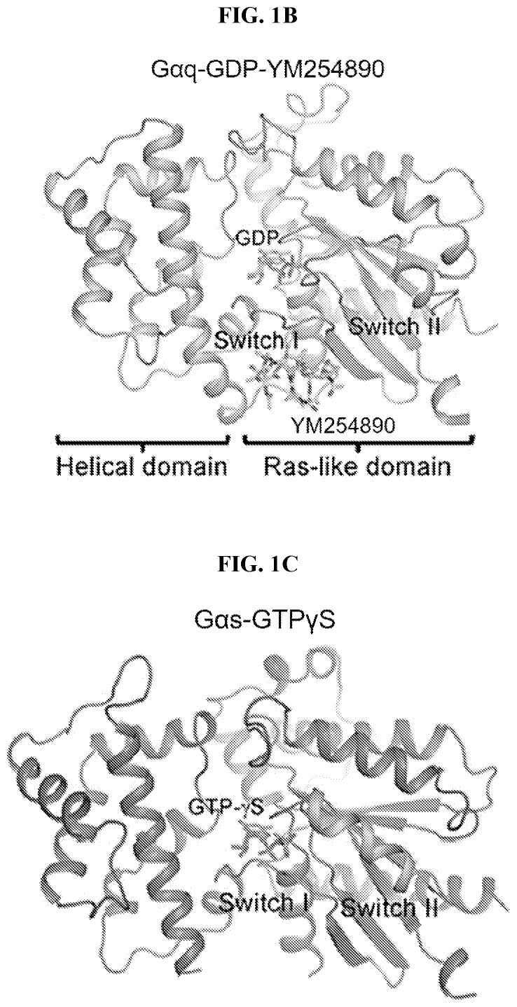

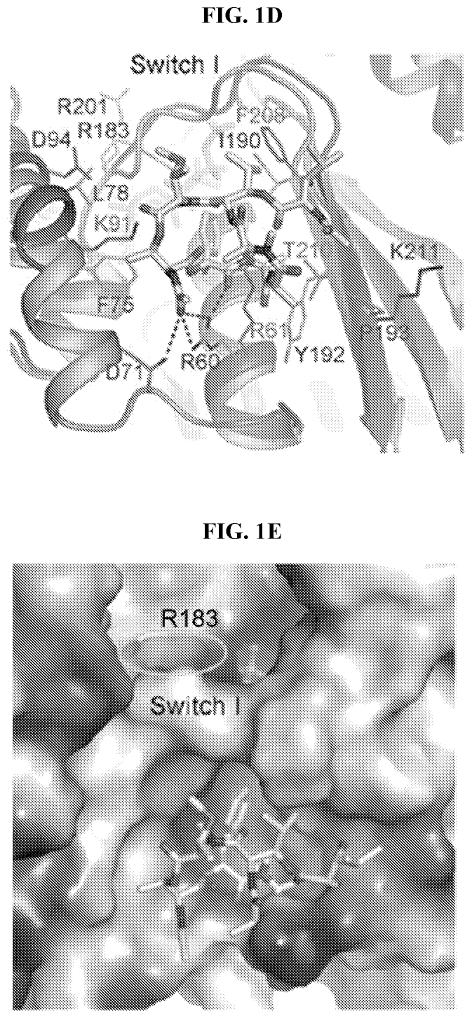

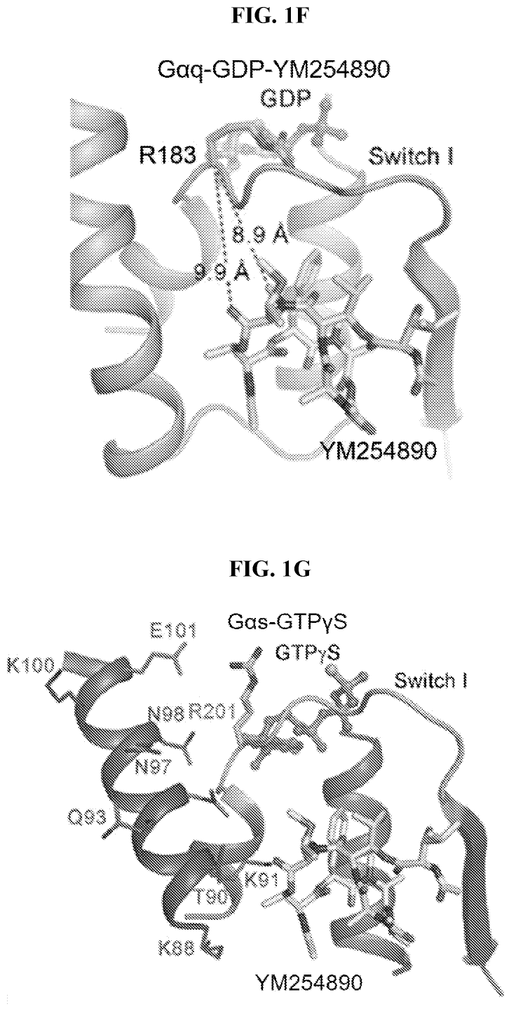

In the crystal structure of Gαq-GDP-YM254890 complex, YM254890 located in a pocket between the Ras-like domain and helical domain (FIG. 1B, PDB code 1AZT). The α-helix and β-sheet that linked by switch I, as well as switch I, surround YM254890, playing an important role for binding. Binding of YM254890 locks Gαq in a GDP-bound state, thus inhibits Gαq activation^{[8]}. Crystal structure of Gαs-GTPγS complex can be superimposed with that of Gαq-GDP-YM254890 complex with a root mean squared deviation (r.m.s.d.) of 2.3 Å (FIG. 1C). A similar pocket surrounded by switch I in Gαs provides potential inhibitor-binding sites.

To design leading compounds that can bind Gαs, I carefully analyzed the interactions between YM254890 and Gαq (FIG. 1D). The phenyl group of YM254890 inserts into a hydrophobic pocket of the Ras-like domain of Gαq. It was reported that replacement of the hydrophobic residues of switch I with hydrophilic residues severely weakened the inhibition effect^{[8]}. Switch I was thus supposed to determine the sensitivity of G-proteins to YM254890^{[8]}. Besides, there are three hydrogen bonds between YM254890 and Arg60 of Gαq, signifying the importance of Arg60. Comparison reveals that most of the key residues are conserved in Gαs. But there are also some obvious differences between Gαs and Gαq that may explain the inability of YM254890 to inhibit Gαs. These differences include K91 vs. F75, D94 vs. L78, F206 vs. 1190, T210 vs. Y192 and K211 vs. P193. Such differences result a less hydrophobic pocket, and also change the shape of the pocket.

Leading compounds that can bind Gαs could obtained by modification of YM254890. Residue (2S,3R)—N,O-Me₂Thr of YM254890 is adjacent to F75 and L78 of Gαq. To change the specificity from Gαq to Gαs, (2S,3R)—N,O-Me₂Thr could be replaced with hydrophilic residues to bind K91 and D94 in Gαs. The isopropyl group of (2S,3R)-β-HyLeu-2 interact with 1190 in Gαq, but replacement of 1190 with F206 in Gαs narrows the pocket. So the isopropyl group could be changed to a smaller group, such as methyl group, to be accommodated by F206. I think such a rational design could help us obtain the leading compounds.

### Modification of the Leading Compounds to Covalently Bond Cys201 of Gαs(R201C)

The aim of this proposal is to develop small molecules to specifically inhibit the cancer-associated mutant Gαs(R201C) but not wild-type Gαs, so the leading compounds should be further modified. Design of specific inhibitors of cancer-associated mutant K-Ras(G12C) provides good examples of using cysteine-reactive small molecules in drug design.

K-Ras, an important member in the Ras family, also has cancer-associated mutants. One of these mutants, G12C, disrupts the GTPase activity of K-Ras, keeping K-Ras in a GTP-bound state. Small molecule inhibitors specifically target the mutant but not wild-type K-Ras have been reported by two groups^{[9-11]}. These inhibitors harbor electrophilic groups that can be covalently linked with Cys12 of the mutant K-Ras. Such electrophilic groups guarantee the specificity of the inhibitors.

Noting that R201C mutation of Gαs is just located in switch I, and is adjacent to the YM254890-binding pocket, I propose to introduce a cysteine-reactive group in the leading compounds to bond Cys201 of the mutant Gαs. Residue (2S,3R)—N,O-Me₂Thr of YM254890 may be a proper position to introduce cysteine-reactive groups. Comparing to other positions on YM254890, (2S,3R)—N,O-Me₂Thr is more close to R183 (corresponding to R201 in Gαs). In addition, a surface grove lays between the helical domain and Ras-like domain; R183 and (2S,3R)—N,O-Me₂Thr can be connected by a linker across this grove (FIG. 1E). Therefore, a linker would be added to the side chain or main chain of (2S,3R)—N,O-Me₂Thr; the other head of the linker would be linked with various cysteine-reactive groups. The length and composition of the linker would be varied to acquire a better inhibitor.

Specifically, a long linker is needed because the distance between the α-carbon of R183 and the main chain of (2S,3R)—N,O-Me₂Thr is nearly 10 Å (FIG. 1F). Considering that there are several hydrophilic residues in the grove between the helical domain and Ras-like domain in Gαs (FIG. 1G), a linker with certain hydrophilicity would be used. Such a linker might also increase the binding affinity of the inhibitor, and fix switch I in an inactive conformation.

As for the cysteine-reactive groups, because cysteine has long been an ideal residue for selective modification of proteins, several classes of reactive groups have been developed to chemically modify cysteine residues, such as α-halocarbonyls (e.g., iodoacetamides), maleimides, vinyl sulfones, etc._{[25, 26]}. Besides, a series of cysteine proteases inhibitors that can covalently modify the cysteine residue have been reported, such as epoxysuccinyl derivatives and O-acyl hydroxamates_{[27]}. These reactive groups can be employed to link the leading compounds with Cys201.

Structural Analysis of the Binding of these Compounds with Gαs(R201C);

Gαs can be easily over-expressed in E. coli^{[28]} and in insect cells^{[29]}. Crystal structures of Gαs-GTPγS complex, Gαs-adenylyl cyclase complex, and the ternary complex of Gαs-Gβγ-β2AR, have been reported^{[7, 13, 29]}. So the crystal structure of Gαs or Gαs(R201C) in complex with its inhibitor may be obtained.

Evaluation of the Potency of these Inhibitors

An in vitro assay system can be used to evaluate the inhibition effects of the designed compounds on the activities of Gαs and the cancer-associated mutant Gαs(R201C). Covalent modification of Gαs(R201C) can be detected by mass spectrometry as described in the study of K-Ras(G12C) inhibitors that carried by Kevan Shokat's laboratory in 2013^{[11]}. After modification, nucleotide exchange rates on Gαs can be determined by a fluorescence-based assay^{[30, 31]}.

### Potential Problems and Alternatives

It might be possible that simple modifications of YM254890 cannot give us the desired Gαs-specific leading compounds, and even if we get the leading compounds, we might fail to introduce a proper linker with a cysteine-reactive group. If so, other strategies would be considered. As I mentioned above, specific inhibitors of K-Ras(G12C) have been reported by two groups. One is Kevan Shokat's group. They have developed a series of compounds with cysteine-reactive groups to covalently modify Cys12 of K-Ras(G12C), while the aryl groups of these compounds bound into a pocket around switch II (not shown, PDB code: 4NMM)^{[11]}. This binding disturbed switch II, and thus convert the nucleotide preference of K-Ras(G12C) to favour GDP over GTP^{[11]}. There is also a similar pocket around switch II in Gαs that has the potential to bind with inhibitors; besides, the location of Cys201 gives it the similar advantage to Cys12 in K-Ras for inhibitor design (not shown, PDB code: 4NMM). The other is Nathanael S. Gray's group. They used GDP analogues to occupy the nucleotide-binding pocket, thus block the entry of GTP; these GDP analogues also contain a cysteine-reactive group to form covalent bond with Cys12 (not shown, PDB code: 4NMM)^{[9, 10]}. So we could employ similar strategies to design Gαs(R201C)-specific inhibitors.

Somatic mutations of GNAS (encoding Gαs) occur in approximately 4.2% of all cancer types^{[1]}. For example, statistics show that 11.8% of 473 pancreas cancer samples and 27.9% of 816 pituitary cancer samples harbor GNAS mutations^{[1]}; 66% of 132 intraductal papillary mucinous neoplasm (IPMN) patients carried a GNAS mutation^{[5]}. These mutations lead to constitutive activation of Gαs, and promote tumourigenesis. About 64% of the cancer-associated mutations of GNAS change Arg201 of Gαs to a cysteine residue (R201C). Therefore, specific inhibitors of the R201C mutant of Gαs would be effective tools for cancer therapies. Development of such inhibitors is just the aim of the proposed research.

### Significance

Somatic mutations of GNAS (encoding Gαs) occur in approximately 4.2% of all cancer types^{[1]}. For example, statistics show that 11.8% of 473 pancreas cancer samples and 27.9% of 816 pituitary cancer samples harbor GNAS mutations^{[1]}; 66% of 132 intraductal papillary mucinous neoplasm (IPMN) patients carried a GNAS mutation^{[5]}. These mutations lead to constitutive activation of Gαs, and promote tumourigenesis. About 64% of the cancer-associated mutations of GNAS change Arg201 of Gαs to a cysteine residue (R201C). Therefore, specific inhibitors of the R201C mutant of Gαs would be effective tools for cancer therapies. Development of such inhibitors is just the aim of the proposed research.

### REFERENCES IN EXAMPLE 1

[1] O'Hayre M, Vazquez-Prado J, Kufareva I, et al. The emerging mutational landscape of G proteins and G-protein-coupled receptors in cancer. Nat Rev Cancer, 2013, 13(6):412-424.
[2] Landis C A, Masters S B, Spada A, et al. GTPase inhibiting mutations activate the alpha chain of Gs and stimulate adenylyl cyclase in human pituitary tumours. Nature, 1989,340(6236):692-696.
[3] Vallar L, Spada A, Giannattasio G. Altered Gs and adenylate cyclase activity in human GH-secreting pituitary adenomas. Nature, 1987,330(6148):566-568.
[4] Lyons J, Landis C A, Harsh G, et al. Two G protein oncogenes in human endocrine tumors. Science, 1990,249(4969):655-659.
[5] Wu J, Matthaei H, Maitra A, et al. Recurrent GNAS mutations define an unexpected pathway for pancreatic cyst development. Sci Transl Med, 2011, 3(92):66r-92r.
[6] Coleman D E, Berghuis A M, Lee E, et al. Structures of active conformations of Gi alpha 1 and the mechanism of GTP hydrolysis. Science, 1994,265(5177):1405-1412.
[7] Sunahara R K, Tesmer J J, Gilman A G, et al. Crystal structure of the adenylyl cyclase activator Gsalpha. Science, 1997,278(5345):1943-1947.
[8] Nishimura A, Kitano K, Takasaki J, et al. Structural basis for the specific inhibition of heterotrimeric Gq protein by a small molecule. Proc Natl Acad Sci USA, 2010,107(31):13666-13671.
[9] Hunter J C, Gurbani D, Ficarro S B, et al. In situ selectivity profiling and crystal structure of SML-8-73-1, an active site inhibitor of oncogenic K-Ras G12C. Proc Natl Acad Sci USA, 2014,111(24):8895-8900.
[10] Lim S M, Westover K D, Ficarro S B, et al. Therapeutic targeting of oncogenic K-Ras by a covalent catalytic site inhibitor. Angew Chem Int Ed Engl, 2014, 53(1):199-204.
[11] Ostrem J M, Peters U, Sos M L, et al. K-Ras(G12C) inhibitors allosterically control GTP affinity and effector interactions. Nature, 2013,503(7477):548-551.
[12] Chung K Y, Rasmussen S G, Liu T, et al. Conformational changes in the G protein Gs induced by the beta2 adrenergic receptor. Nature, 2011,477(7366):611-615.
[13] Tesmer J J, Sunahara R K, Gilman A G, et al. Crystal structure of the catalytic domains of adenylyl cyclase in a complex with Gsalpha.GTPgammaS. Science, 1997,278(5345):1907-1916.
[14] Weinstein L S, Shenker A, Gejman P V, et al. Activating mutations of the stimulatory G protein in the McCune-Albright syndrome. N Engl J Med, 1991,325(24):1688-1695.
[15] Mukai H, Munekata E, Higashijima T. G protein antagonists. A novel hydrophobic peptide competes with receptor for G protein binding. J Biol Chem, 1992,267(23):16237-16243.
[16] Ayoub M A, Damian M, Gespach C, et al. Inhibition of heterotrimeric G protein signaling by a small molecule acting on Galpha subunit. J Biol Chem, 2009,284(42):29136-29145.
[17] Schmitz A L, Schrage R, Gaffal E, et al. A cell-permeable inhibitor to trap Galphaq proteins in the empty pocket conformation. Chem Biol, 2014, 21(7):890-902.
[18] Takasaki J, Saito T, Taniguchi M, et al. A novel Galphaq/11-selective inhibitor. J Biol Chem, 2004,279(46):47438-47445.
[19] Freissmuth M, Boehm S, Beindl W, et al. Suramin analogues as subtype-selective G protein inhibitors. Mol Pharmacol, 1996, 49(4):602-611.
[20] Hohenegger M, Waldhoer M, Beindl W, et al. Gsalpha-selective G protein antagonists. Proc Natl Acad Sci USA, 1998, 95(1):346-351.
[21] Chung W C, Kermode J C. Suramin disrupts receptor-G protein coupling by blocking association of G protein alpha and betagamma subunits. J Pharmacol Exp Ther, 2005,313(1):191-198.
[22] Milburn M V, Tong L, Devos A M, et al. Molecular switch for signal transduction: structural differences between active and inactive forms of protooncogenic ras proteins. Science, 1990,247(4945):939-945.
[23] Taniguchi M, Nagai K, Arao N, et al. YM-254890, a novel platelet aggregation inhibitor produced by Chromobacterium sp. QS3666. J Antibiot (Tokyo), 2003, 56(4):358-363.
[24] Taniguchi M, Suzumura K, Nagai K, et al. Structure of YM-254890, a novel G q/11 inhibitor from Chromobacterium sp. QS3666. Tetrahedron, 2003, 59(25):4533-4538.
[25] Chalker J M, Bernardes G J, Lin Y A, et al. Chemical modification of proteins at cysteine: opportunities in chemistry and biology. Chem Asian J, 2009, 4(5):630-640.
[26] Spicer C D, Davis B G. Selective chemical protein modification. Nat Commun, 2014, 5:4740.
[27] Otto H H, Schirmeister T. Cysteine Proteases and Their Inhibitors. Chem Rev, 1997, 97(1):133-172.
[28] Lee E, Linder M E, Gilman A G. Expression of G-protein alpha subunits in Escherichia coli. Methods Enzymol, 1994, 237:146-164.
[29] Rasmussen S G, Devree B T, Zou Y, et al. Crystal structure of the beta2 adrenergic receptor-Gs protein complex. Nature, 2011,477(7366):549-555.
[30] Kimple R J, Jones M B, Shutes A, et al. Established and emerging fluorescence-based assays for G-protein function: heterotrimeric G-protein alpha subunits and regulator of G-protein signaling (RGS) proteins. Comb Chem High Throughput Screen, 2003, 6(4):399-407.
[31] Mcewen D P, Gee K R, Kang H C, et al. Fluorescent BODIPY-GTP analogs: real-time measurement of nucleotide binding to G proteins. Anal Biochem, 2001,291(1):109-117.

### Example 2. In Vitro Assays

Measuring the GDP Dissociation Rate of Gαs R201C is Slower than its GTP Hydrolysis Rate

The R201C mutant Gαs can bypass the need for GTP binding by directly activating GDP-bound Gαs through stabilization of an intramolecular hydrogen bond network between the P-loop, switch III and switch II of Gαs. In order to check whether the R201C mutant in cells would be mostly in the GTP-bound form thus rendering our biochemical findings about the active GDP-bound not physiologically relevant, the single turnover GTP hydrolysis rate _{(kcat)} and the GDP dissociation rate (k_{off}) of R201C and wild-type Gαs were measured and compared. The ratio of Gαs in the GTP-bound state should be less than k_{off}/(k_{off}+_{kcat}) when the GTP binding and hydrolysis cycle reaches a steady state. In the presence of excess G_{f3}γ subunits and millimolar Mg²⁺, only 11% of the R201C mutant was in the GTP state without stimulation by GPCRs.

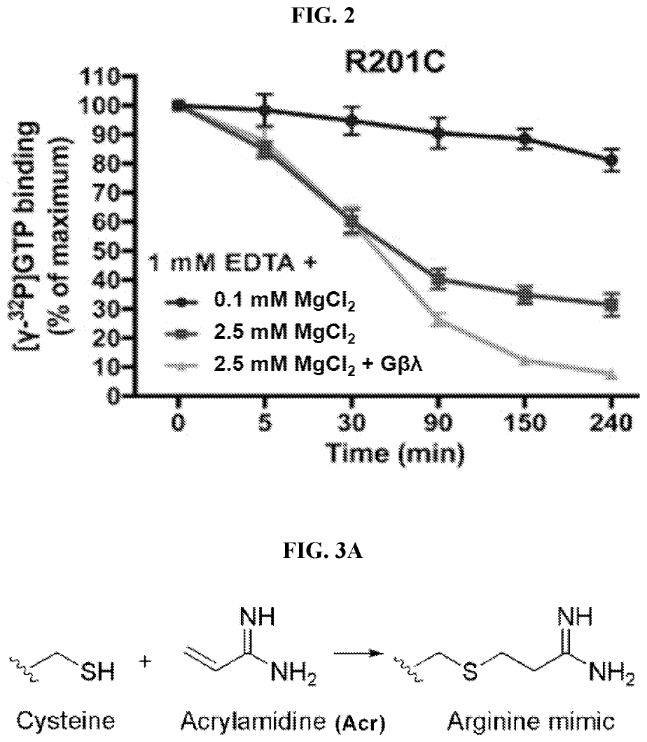

The calculation using a [γ-³²P]GTP binding assay is shown in FIG. 2. The R201C mutant was pre-incubated in a low Mg²⁺ buffer (1 mM EDTA+0.1 mM MgCl₂) with 400 μM GTP that is close to the physiological concentration of GTP; 20 nM [γ-³²P]GTP was added as an internal standard. After the binding of [γ-³²P]GTP to the R201C mutant reached a maximum, the concentration of free Mg²⁺ was increased to about 1.1 mM (1 mM EDTA+2.5 mM MgCl₂) and the changes of bound [γ-³²P]GTP with time were measured. The bound [γ-³²P]GTP decreased to about 30% of the maximum after 4 hours, which can be explained by the faster GTP hydrolysis than GDP dissociation. When G_{f3}γ subunits were added together with MgCl₂(G_{f3}γ:Gαs=1.5:1, molar ratio), the bound [γ-³²P]GTP further decreased to below 10% of the maximum after 4 hours, which supports the finding that G_{f3}γ subunits decrease the rate of GDP dissociation. In contrast, when the free Mg²⁺ concentration was kept at 0.1 μM (1 mM EDTA+0.1 mM MgCl₂), the bound [γ-³²P]GTP only slowly decreased to about 80% of the maximum, which may be due to the instability of the R201C mutant in the low Mg²⁺ buffer.

The two pieces of evidence demonstrate that the R201C mutant is not locked in the GTP state, instead, without GPCR stimulation it would be mainly in the GDP state in cells considering that the presence of G_{f3}γ subunits and millimolar Mg²⁺ dramatically decrease the rate of GDP dissociation.

### Correction of the Misactivation of Gαs(R201C) by an Arginine Mimic

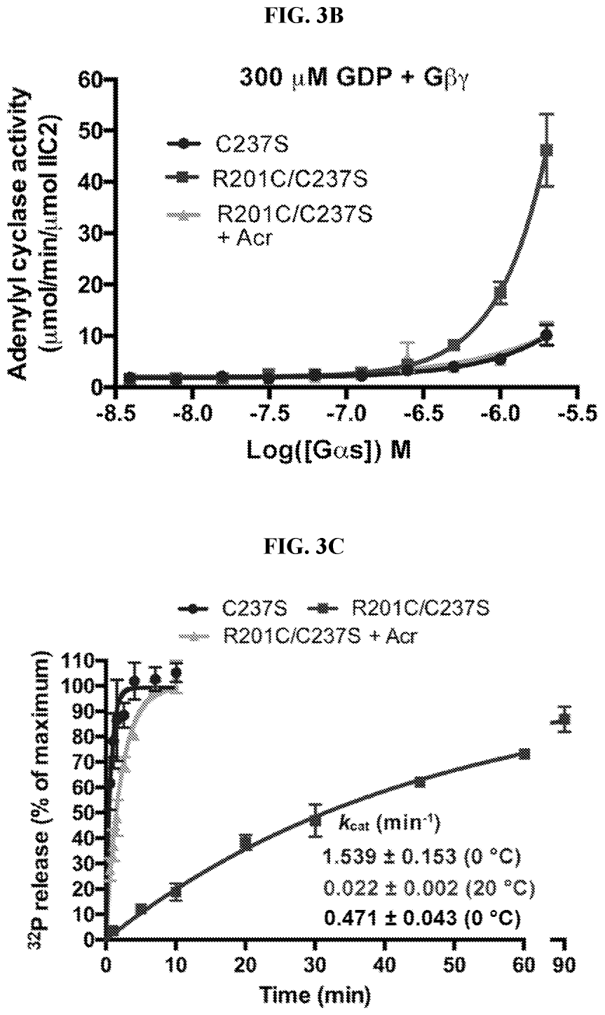

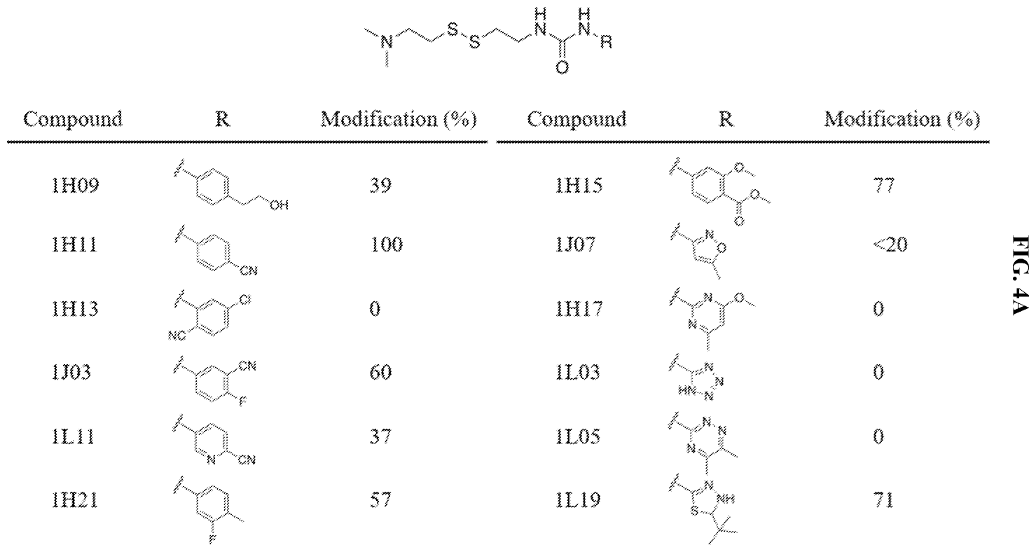

A crystal structure of the R201C/C237S mutant was solved and shown in FIG. 1A. Structure analysis indicates the importance of the interactions between E50 and ammonium η1 and η2 of R201 in maintaining GDP-bound Gαs in an inactive state. The R201C mutation results in the loss of ammonium η1 and η2 of R201 thus freeing E50 to interact with R258 and R265 to indirectly stabilize switch II in an active-like conformation. Based on this finding, replacing the side chain of R201C with a close mimic of native arginine which contains N(η1) and N(η2) may correct the misactivation of the R201C mutant. For example, acrylamidine (Acr) can modify Cys237 and Cys201 in the R201C mutant. In the R201C/C237S double mutant, only Cys201 can be modified by Acr. This modification converts cysteine to an arginine mimic (FIG. 3A).

Also effect of modifying C201 with Acr on the adenylyl cyclase-activating activity of GDP-bound Gαs(R201C/C237S) was calculated. In the presence of GDP and Gβ1/Gγ2(C68S), the unmodified Gαs(R201C/C237S) showed significantly higher activity than Gαs(C237S) (FIG. 3B), consistent with our finding that Gαs(R201C) has a higher activity than WT Gαs. After Gαs(R201C/C237S) was modified by Acr (free Acr was removed by gel filtration), its ability to activate adenylyl cyclase was lowered to the same level as that of Gαs(C237S) (FIG. 3B). Particularly, site specific modification of C201 with Acr can effectively restore the canonical role of GDP to Gαs disrupted by the R201C mutation, supporting the role of N(η1) and N(η2) in restraining the GDP-bound form in a state that does not activate adenylyl cyclase in the presence of Gβγ subunits. It is also demonstrated that this modification can partly restore the GTPase activity of Gαs(R201C/C237S). The k_{cat} of Gαs(C237S) is 1.539±0.153 min⁻¹ (measured at 0° C.), slightly higher than that of WT Gαs (FIG. 3C). The unmodified R201C/C237S mutant showed a slow k_{cat} (0.022±0.002 min⁻¹) even at 20° C., similar to that of Gαs(R201C). Following Acr modification of the R201C/C237S mutant its k_{cat} increased to 0.471±0.043 min⁻¹ (measured at 0° C.), about 30% of that of Gαs(C237S). These results show the possibility of correction of the misactivation of Gαs(R201C) through covalently modifying C201 with compounds that can mimic the function of arginine.

Identification of Lead Compounds that Covalently Modify C201 in Gαs (R201C)

A tethering screening was used to identify compounds that can covalently modify C201. Untagged recombinant Gαs(R201C) at 2 μM was reacted with 200 μM fragment and 200 μM βME in 20 mM HEPES, pH 8.0, 150 mM NaCl, 30 μM MgCl₂, 50 μM GDP for 2 h at ambient temperature. The extent of modification was assessed by electrospray mass spectrometry using a Waters LCT-Premier LC/ESI-MS.

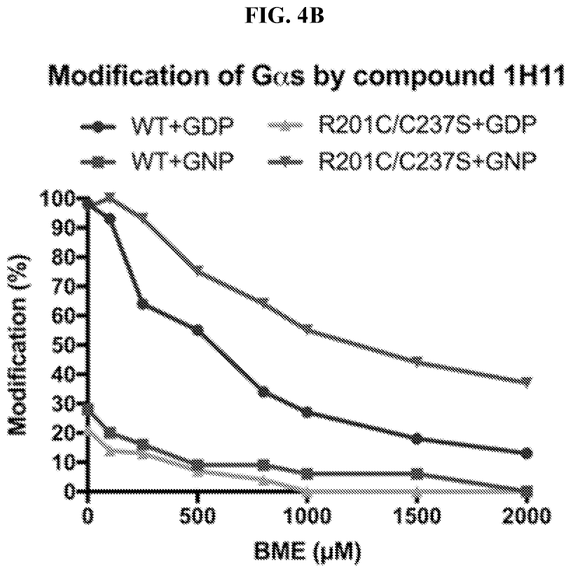

In the related studies, a native cysteine, C237, can be more reactive than C201 so that all the compounds that efficiently modified Gαs(R201C) were finally proved to target C237 but not C201. Therefore, instead of GDP-bound Gαs(R201C) the double mutant Gαs(R201C/C237S) was used this time. After screening about 1600 disulphide fragments, a class of compounds that modified Gαs(R201C/C237S) but not Gαs(C237S) was identified. These compounds are distinguished by containing a urea moiety (FIG. 4A) that likely mimics the guanidine group in arginine. Among them, compound 1H11 gave the highest modification ratio.

The reactivity of C201 and C237 against compound 1H11 were compared. Not surprisingly, when the concentration of the compound was lowered from 200 μM to 50 μM, compound 1H11 only modified about 20% of the GDP-bound Gαs(R201C/C237S) even when BME (beta mercaptoethanol) concentration was 0 while modified nearly 100% of GDP-bound wild-type Gαs in the same condition (FIG. 4B, circle and triangle curves), indicating that in the GDP-bound state C237 is more reactive than C201 against this compound. But when GDP in Gαs was replaced with GNP (5-Guanylyl imidodiphosphate, a non-hydrolyzable GTP analog), the reactivity of C201 was dramatically increased, with a modification ratio over 50% even at a BME concentration of 1000 μM (FIG. 4B, reverse triangle curve); in contrast, the reactivity of C237 was much lower (FIG. 4B, square curve). These results demonstrate that compound 1H11 prefers C201 in the GNP-bound Gαs. Such a preference makes 1H11 a good lead compound for the development of inhibitors that selectively target the active GTP-bound Gαs. One possibility is that the γ-phosphate in GNP directly binds to the urea moiety in compound 1H11. To get information to optimize 1H11, the crystal structure of the GNP-bound Gαs(R201C/C237S) in complex with 1H11 and effects of 1H11 on the GTPase activity and adenylyl cyclase-activating activity of Gαs would be obtained.

Chemotherapy is one of the mainstays of cancer treatment. However, this approach is often troubled by severe side effects mainly because anti-cancer drugs target both cancer cells and healthy cells. Cancer is caused by mutations in genes that accelerate (oncogenes) or suppress (tumor suppressors) tumor growth. Specifically targeting the mutant genes or the mutant proteins encoded by these genes is theoretically an ideal strategy to decrease the side effects of anti-cancer drugs but usually cannot be achieved. In the cases that the cancer-causing mutations result in a cysteine residue in the mutation site, inhibitors that covalently modify the cysteine residue have shown great selectivity of the mutant proteins over the normal proteins. Indeed, activating mutations in an oncogene called GNAS that encodes the protein Gαs were identified in 27.9% of 816 pituitary cancer samples and 66% of a type of pancreatic cancer patients. More than half of these mutations result in the substitution of Arg201 of Gαs by a cysteine residue. In recent research, a small molecule named acrylamidine can correct the misactivation of Gαs(R201C) by converting Cys201 to an arginine mimic. Also identified is a disulphide fragment that preferentially modifies Cys201 in the active GTP-bound form of Gαs. Both small molecules are not drug-like molecules, however, they are good starting point for development of drug-like molecules.

### Method and Material

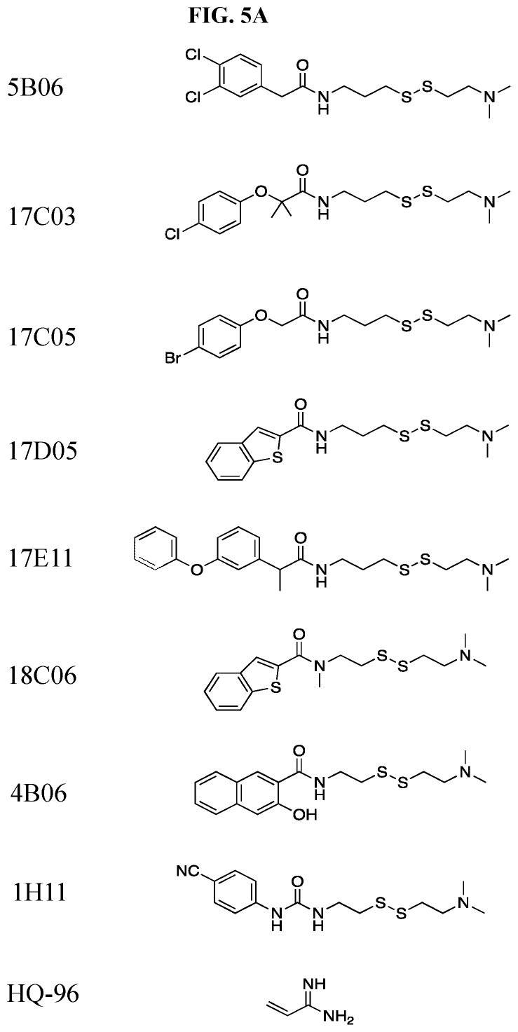

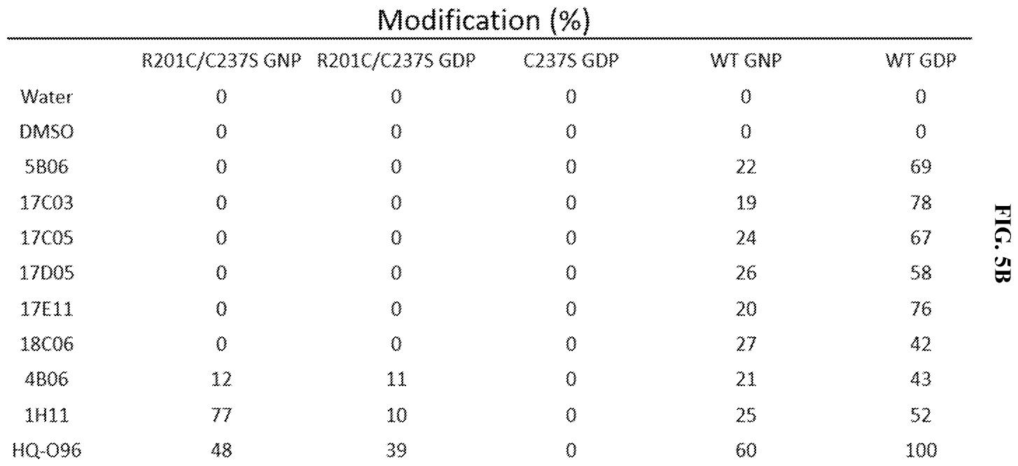

Compounds shown in FIG. 5A selected from the tethering library were tested for their ability to label different Gαs mutants. Modification rate by these compounds are shown in FIG. 5B.

Proteins were diluted in 20 mM HEPES 8.0, 150 mM NaCl, 5 mM MgCl₂, 1 mM EDTA as indicated in Table 1.

TABLE 1

| Protein types | concentration |
|---|---|
| GNP-bound R201C/C237S | 12 mg/mL |
| GDP-bound R201C/C237S mutant | 12 mg/mL |
| GDP-bound C237S mutant | 12 mg/mL |
| GNP-bound WT | 10 mg/mL |
| GDP-bound WT | 8.5 mg/mL |

The compounds of covalent bond mimic from the tethering library are shown in FIG. 5A. These compounds were diluted to 2 μM using 20 mM HEPES 7.5, 150 mM NaCl, 5 mM MgCl₂ and solutions of final sample volume of 700 μL were obtained. For each sample, 50 μL was used as control, 50 μL was mixed with 100 μM compound HQ-96 (5 mM stock in H₂O), 600 μL was mixed with 0.5 mM BME (100 mM stock in H₂O). Then, 50 μL of each sample that mixed with BME was incubated with 100 μM compound 5B06, 17C03, 17C05, 17D05, 17E11, 18C06, 4B06, 1H11, DMSO, or HQ-30 (5 mM stock in DMSO). The samples were incubated at room temperature for 2 hours and the modification rate of the proteins by these small molecules was checked by LC/MS.

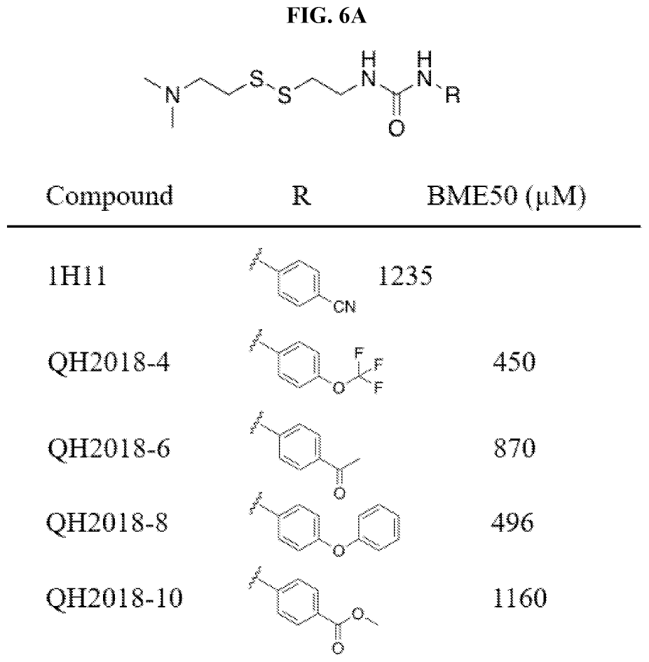

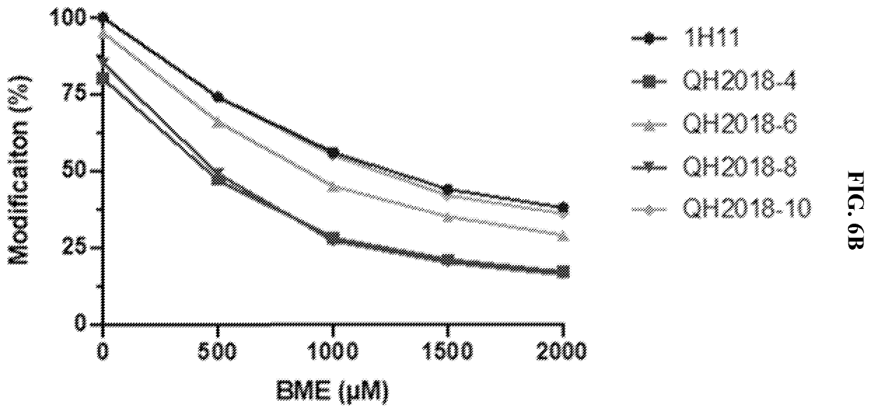

In addition, compounds of covalent bond mimic in FIG. 6A containing an aryl urea moiety were tested for their BME50 values (FIG. 6B). Proteins (GNASSd6 R201C/C237S) in a buffer solution (8.5 mg/mL in 150 mM NaCl, 20 mM HEPES 8.0, 5 mM MgCl₂, 1 mM EDTA-Na (pH 8.0) were diculted) to 2 μM using an EDTA containing buffer (150 mM NaCl, 20 mM HEPES 7.5, 1 mM EDTA). The diluted protein solutions were incubated with 0.5 mM GMPPNP at room temperature for 1 hour, then MgCl₂ was added to a final concentration of 5 mM. BME was added to a final concentration of 0, 500, 1000, 1500 or 2000 μM. Compounds QH2018-4, QH2018-6, QH2018-8 or QH2018-10 (5 mM in DMSO) was added to each sample to a final concentration of 50 μM. After incubation at room temperature for 2 hours, labeling ratio of the proteins by these compounds was determined by LC/MS. The measured BME50 are shown in FIG. 6B.

### Example 3. Synthesis of Compounds

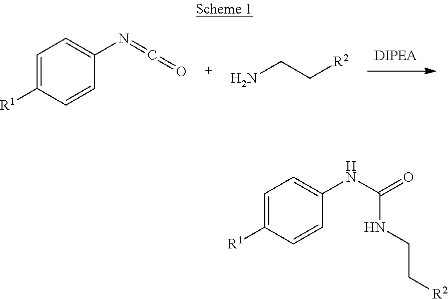

Reactive aryl isocyate with the R¹ substitution as described herein on para position and aliphatic amine with the R² substituents as described herein may form an aryl urea upon reaction illustrated in Scheme 1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound having the formula: 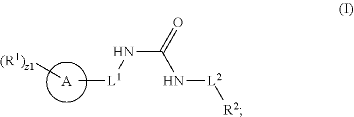
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 6;
Ring A is aryl or heteroaryl;
L¹ is a bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R² is an electrophilic moiety;
R^{1A}, R^{1B}, R^{1C}, and R^{1D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X and X¹ are independently —F, —Cl, —Br, or —I;
n1 is independently an integer from 0 to 4; and
m1 and v1 are independently 1 or 2.

2.-4. (canceled)

5. The compound of claim 1, having the formula: 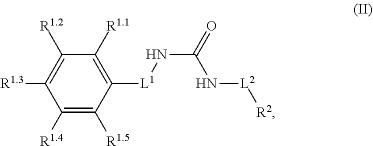
wherein;
each R^{1.1}, R^{1.2}, R^{1.3}, R^{1.4}, and R^{1.5} is independently hydrogen, halogen, —CX¹₃, —CHX¹₂, —CH₂X¹, —OCX¹₃, —OCH₂X¹, —OCHX¹₂, —CN, —SO₁₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X and X¹ are independently —F, —Cl, —Br, or —I;
n1 is independently an integer from 0 to 4; and
m1 and v1 are independently 1 or 2.

6. (canceled)

7. The compound of claim 1, wherein L¹ is a bond, —NH—, —O—, —S—, —C(O)—, —C(O)NH—, —NHC(O)—, —NHC(O)NH—, —C(O)O—, —OC(O)—, substituted or unsubstituted C₁-C₆ alkylene, or substituted or unsubstituted 2 to 6 membered heteroalkylene.

8. (canceled)

9. (canceled)

10. The compound of claim 1, wherein L² is a bond, —NH—, —O—, —S—, —C(O)—, —C(O)NH—, —NHC(O)—, —NHC(O)NH—, —C(O)O—, —OC(O)—, substituted or unsubstituted C₁-C₆ alkylene, or substituted or unsubstituted 2 to 6 membered heteroalkylene.

11. (canceled)

12. (canceled)

13. The compound of claim 1, wherein R² is 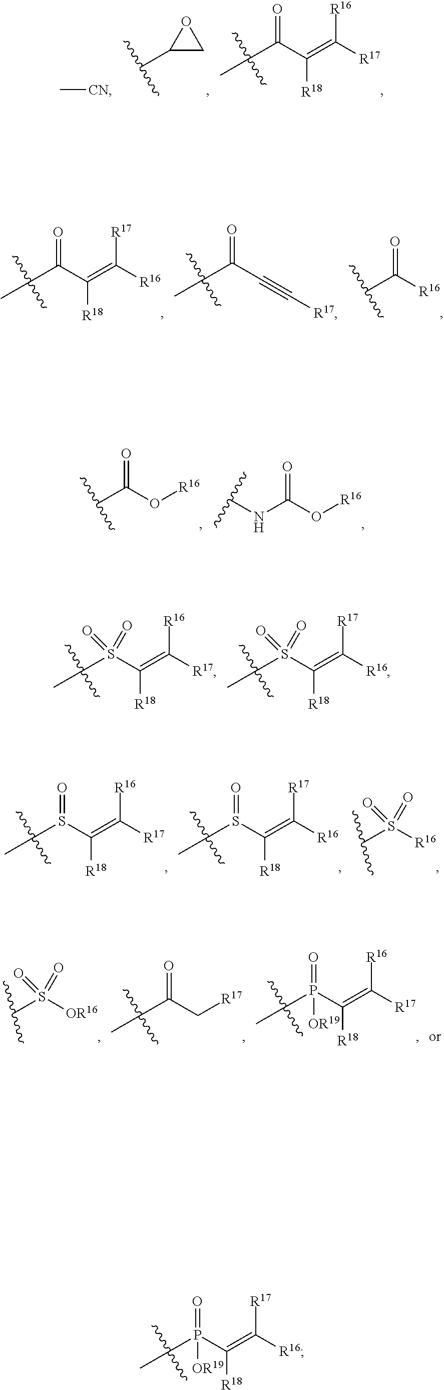
R¹⁶ is hydrogen, halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16B}, —NR^{16A}C(O)R^{16C}, —NR^{16A}C(O)OR^{16C}, —NR^{16A}OR^{16D}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is hydrogen, halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17B}, —NR^{17A}C(O)R^{17C}, —NR^{17A}C(O)OR^{17C}, —NR^{17A}OR^{17D}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is hydrogen, halogen, —CX¹⁸₃, —CHX¹⁸₂, —CH₂X¹⁸, —CN, —SOₙ₁₈R^{18D}, —SOᵥ₁₈NR^{18A}R^{18B}, —NHNR^{18A}R^{18B}, —ONR^{18A}R^{18B}, —NHC(O)NHNR^{18A}R^{18B}, —NHC(O)NR^{18A}R^{18B}, —N(O)ₘ₁₈, —NR^{18A}R^{18B}, —C(O)R^{18C}, —C(O)—OR^{18C}, —C(O)NR^{18A}R^{18B}, —OR^{18D}, —NR^{18A}SO₂R^{18B}, —NR^{18A}C(O)R^{18B}, —NR^{18A}C(O)OR^{18C}, —NR^{18A}OR^{18D}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁹ is hydrogen, halogen, —CX¹⁹₃, —CHX¹⁹₂, —CH₂X¹⁹, —CN, —SOₙ₁₉R^{19D}, —SOᵥ₁₉NR^{19A}R^{19B}, —NR^{19A}R^{19B}, —ONR^{19A}R^{19B}, —NHC(O)NHNR^{19A}R^{19B}, —NHC(O)NR^{19A}R^{19B}, —N(O)ₘ₁₉, —NR^{19A}R^{19B}, —C(O)R^{19C}, —C(O)—OR^{19C}, —C(O)NR^{19A}R^{19B}, —OR^{19D}, —NR^{19A}SO₂R^{19B}, —NR^{19A}C(O)R^{19C}, —NR^{19A}C(O)OR^{19C}, —NR^{19A}OR^{19D}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, and R^{19D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
X¹⁶, X¹⁷, X¹⁸, and X¹⁹ are independently —F, —Cl, —Br, or —I;
n16, n17, n18, and n19 are independently an integer from 0 to 4; and
m16, m17, m18, m19, v16, v17, v18, and v19 are independently 1 or 2.

14. (canceled)

15. The compound of claim 13, wherein
R¹⁶ is hydrogen, unsubstituted C₁-C₄ alkyl, or unsubstituted C₃-C₆ cycloalkyl;
R¹⁷ is hydrogen, unsubstituted C₁-C₄ alkyl, or unsubstituted C₃-C₆ cycloalkyl; and
R¹⁸ is hydrogen, unsubstituted C₁-C₄ alkyl, or unsubstituted C₃-C₆ cycloalkyl.

16. (canceled)

17. (canceled)

18. A pharmaceutical composition comprising the compound of claim 1 and a pharmaceutically acceptable excipient.

19. A method of inhibiting Gαs protein activity, said method comprising: contacting the Gαs protein with the compound of claim 1.

20. A method of treating cancer, said method comprising administering to a subject in need thereof an effective amount of compound of claim 1.

21. The method of claim 20, wherein the cancer is pancreatic cancer, a pituitary tumor, or a bone tumor.

22. (canceled)

23. A method of treating a bone condition, said method comprising administering to a subject in need thereof an effective amount of the compound of claim 1.

24. The method of claim 23, wherein the bone condition is fibrous dysplasia.

25. (canceled)

26. A method of treating McCune-Albright Syndrome, said method comprising administering to a subject in need thereof an effective amount of the compound of claim 1.

27. A Gαs protein covalently bonded to the compound of claim 1.

28.-30. (canceled)

31. The Gαs protein of claim 27, having the structure: 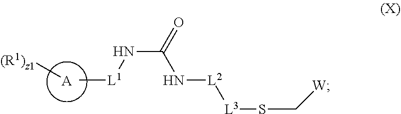
wherein,
W together with the —CH₂S— to which it is attached form said Gαs protein covalently bonded to the compound; and
L³ is substituted or unsubstituted alkylene or substituted or unsubstituted heteroalkylene.

32.-34. (canceled)

35. A Gαs protein covalently bonded to a portion of the compound of claim 1.

36. A Gαs protein covalently bonded to a Gαs small molecule inhibitor at R201C.

37. (canceled)

38. (canceled)

39. A Gαs protein covalently bonded to a Gαs small molecule inhibitor at C237.

40. (canceled)

41. (canceled)

42. A method of treating cancer comprising administering a Gαs cysteine 201 covalent inhibitor.

43. (canceled)

44. A method of treating cancer comprising administering a Gαs cysteine 237 covalent inhibitor.

45. (canceled)
